(19) United States
(12) Patent Application Publication
(10) Pub. No.: US 2024/0335511 A1
(43) Pub. Date: Oct. 10, 2024

(54) THERAPEUTIC RNA FOR TREATING CANCER

(71) Applicant: BioNTech SE, Mainz (DE)

(72) Inventors: Ugur SAHIN, Mainz (DE); Alexander MUIK, Mainz (DE); Lena Mareen KRANZ, Mainz (DE); Mathias VORMEHR, Mainz (DE); Sina FELLERMEIER-KOPF, Mainz (DE); Jan DIEKMANN, Mainz (DE); David EISEL, Mainz (DE)

(21) Appl. No.: 18/268,124

(22) PCT Filed: Dec. 20, 2021
(86) PCT No.: PCT/EP2021/086778

§ 371 Date: Jun. 16, 2023

(30) Foreign Application Priority Data
Dec. 21, 2020 (WO) PCT/EP2020/087469

(51) Int. Cl. A61K 38/20; A61K 9/51; A61K 38/38; A61K 48/00; A61P 35/00; C07K 14/54; C07K 14/55; C07K 14/765

(52) U.S. Cl. CPC A61K 38/2046; A61K 9/5146; A61K 38/2013; A61K 38/38; A61K 48/0041; A61P 35/00; C07K 14/5418; C07K 14/55; C07K 14/765; C07K 2319/31

(57) ABSTRACT

This disclosure relates to the field of therapeutic RNA to treat cancer, in particular advanced solid tumors such as metastatic (Stage IV) or unresectable localized cancer. Disclosed herein are compositions, uses, and methods for treatment of cancers. Administration of therapeutic RNAs to a patient having cancer disclosed herein can reduce tumor size, prolong time to progressive disease, and/or protect against metastasis and/or recurrence of the tumor and ultimately extend survival time.

This disclosure relates to the field of therapeutic RNA to treat cancer, in particular advanced solid tumors such as metastatic (Stage IV) or unresectable localized cancer. Disclosed herein are compositions, uses, and methods for treatment of cancers. Administration of therapeutic RNAs to a patient having cancer disclosed herein can reduce tumor size, prolong time to progressive disease, and/or protect against metastasis and/or recurrence of the tumor and ultimately extend survival time.

### BACKGROUND

Cancer is the second leading cause of death globally and is expected to be responsible for an estimated 9.6 million deaths in 2018. In general, once a solid tumor has metastasized, with a few exceptions such as germ cell and some carcinoid tumors, 5-year survival rarely exceeds 25%.

Refinements in conventional therapies such as chemotherapy, radiotherapy, surgery, and targeted therapies, and recent advances in immunotherapies have improved outcomes in patients with advanced solid tumors. In the last few years, the FDA and European Medicines Agency (EMA) have approved 6 checkpoint inhibitors (CPIs): ipilimumab, a monoclonal antibody targeting the cytotoxic T lymphocyte-associated protein 4 (CTLA-4) pathway, and 6 monoclonal human antibodies targeting programmed death protein 1 (PD-1)/programmed death ligand 1 (PD-L1), namely atezolizumab, avelumab, durvalumab, nivolumab, cemiplimab and pembrolizumab, for the treatment of patients with multiple cancer types, mainly solid tumors (Gentzler R et al., Immunotherapy 2016; 8(5): 583-600; Ribas A and Wolchok J D, Science. 2018; 359(6382): 1350-55). These approvals have dramatically changed the landscape of cancer treatment. However, the majority of cancer patients, including a large proportion with tumors considered ‘sensitive’ to these agents (e.g., melanoma, non-small cell lung cancer, urothelial carcinoma, kidney cancer, and others), either do not respond or become resistant to these agents (Arora S et al., Adv Ther 2019; 36(10): 2638-78). In addition, some of the most prevalent tumors, colorectal cancer, breast cancer and prostate cancer, have proven to be largely refractory to checkpoint inhibition (Borcherding N et al., J Mol Biol 2018; 430(14): 2014-29).

Many immunotherapy treatments have demonstrated efficacy in only a selected subgroup of cancers, such as those expressing PD-L1 (EMA/533341/2019. An overview of Tecentriq and why it is authorized in the EU [Internet]. European medicines agency. 2019 [cited 2020 May 7]. Available from: https://www.ema.europa.eu/en/documents/overview/tecentriq-epar-medicine-overview_en.pdf), with microsatellite instability/mismatch repair deficiency or high tumor mutational burden (Luchini C et al., Ann Oncol 2019; 30(8): 1232-43). Indeed, despite considerable early success and fewer side effects compared to other systemic therapies, the majority of cancer patients do not respond to CPIs or novel targeted therapies (Arora S et al., Adv Ther 2019; 36(10): 2638-78).

With cures remaining scarce in patients with advanced solid tumors, there is an urgent unmet medical need for more effective and less toxic therapies, in particular those that might have synergistic mechanisms of action with immune CPIs.

IL-7 plays an important role in T and B cell lymphopoiesis and survival as well as memory T cell formation (Fry T J, Mackall C L. Interleukin (IL)-7: from bench to clinic. Blood [Internet]. 2002 Jun. 1; 99(11): 3892-904. Available from =http://www.ncbi.nlm.nih.gov/pubmed/12010786, Cui G et al., Cell 2015; 161(4): 750-61). Injection of recombinant IL-7 was shown to expand CD8⁺ and CD4⁺ T cells while leading to a relative decrease of regulatory T cells (Treg) in humans (Rosenberg S A et al., J Immunother 2006; 29(3): 313-19). Studies in tumor-bearing mice suggest that IL-7 administration supports the anti-tumor effector function of T cells leading to reduced tumor growth (Komschlies K L et al., J Immunol 1994; 152(12): 5776-84). Recombinant IL-7 has been tested extensively not only in cancer patients but also for the treatment of immunodeficiency secondary to organ transplantation, human immunodeficiency virus (HIV) or septic shock (Francois B et al., JCI insight 2018; 8: 3(5), Thiébaut R et al., Clin Infect Dis 2016; 62(9): 1178-85, Lundström W et al., Semin Immunol 2012; 24(3): 218-24, Trédan O et al., Ann Oncol 2015; 26(7): 1353-62). Recombinant IL-7 has been described to be well tolerated in humans, with side effects comprising mild and transient fever (Rosenberg S A et al., J Immunother 2006; 29(3): 313-19, Trédan O et al., Ann Oncol 2015; 26(7): 1353-62, Sportès C et al., Clin Cancer Res 2010; 16(2): 727-35). Recombinant IL-7 has a short plasma half-life in the range of h and therefore requires frequent dosing (Sportès C et al., Clin Cancer Res 2010; 16(2): 727-35).

hIL-2 is a key cytokine in T cell immunity. It supports the differentiation, proliferation, survival and effector functions of T cells (Gillis S, Smith K A, Nature 1977; 268(5616): 154-56, Blattman J N et al., Nat Med 2003; 9(5): 540-47, Bamford R N et al., Proc Natl Acad Sci USA. 1994; 91(11): 4940-44, Kamimura D, Bevan M J, J Exp Med 2007; 204(8): 1803-12). Recombinant rIL-2, aldesleukin, was the first approved cancer immunotherapy and has been used for decades in the treatment of late stage malignant melanoma and renal cell cancer (Kammula U S et al., Cancer 1998; 83(4): 797-805). Most patients with complete responses after rIL-2 treatment remain regression free for more than 25 years after initial treatment, but overall response rates are low (Klapper J A et al., Cancer 2008; 113(2): 293-301, Rosenberg S A et al., Ann Surg 1998; 228(3): 307-19). A particular challenge of rIL2 for cancer treatment is the preferential stimulation of Tregs, which even at low doses dampens anti-tumor immune responses. It requires high rIL-2 doses to efficiently stimulate the intended target population of CD8+ and CD4+ effector T cells (Todd J A et al., PLOS Med 2016; 13(10): e1002139). Recombinant IL-2 has a very short half-life in the range of minutes and therefore requires high and frequent dosing which in turn potentiates its side effects (Kammula U S et al., Cancer 1998; 83(4): 797-805, Todd J A et al., PLOS Med 2016; 13(10): e1002139).

Capillary leak syndrome (CLS) is the main dose-limiting toxicity (Baluna R, Vitetta E S, Immunopharmacology 1997; 37(2-3): 117-32). CLS usually occurs 3 to 4 d after IL-2 treatment and results in decreased microcirculatory perfusion and interstitial edema especially in lung and liver. CLS can lead to multi-organ failure. Most CLS symptoms, however, disappear within 2 weeks after treatment cessation. The exact cause of CLS is only partially understood. It is believed that pro-inflammatory cytokines produced by rIL-2 activated natural killer (NK) cells play an essential role (Assier E et al., J Immunol 2004; 172(12): 7661-68). In addition, a direct effect of rIL-2 on lung endothelial cells has been suggested (Krieg C et al., Proc Natl Acad Sci USA. 2010; 107(26): 11906-11). Other frequently reported side effects are hypotension, diarrhea, oliguria, chills, vomiting, dyspnea, rash, bilirubinemia, thrombocytopenia, nausea, confusion, creatinine increase, anemia, fever, peripheral edema, and malaise (Proleukin* Prescribing Information 2012). Decades of experience with rIL-2 treatment has improved the management of adverse events. Most side effects are easily managed by experienced personnel with the majority of toxicities being reversible upon treatment discontinuation. In addition, established screening guidelines have reduced the risk of treatment-related mortality to essentially zero (Marabondo S, Kaufman H L, Expert Opin Drug Saf 2017; 16(12): 1347-57).

### SUMMARY

The present invention generally embraces the immunotherapeutic treatment of a subject comprising the administration of (i) RNA encoding an amino acid sequence comprising human IL7 (hIL7), a functional variant thereof, or a functional fragment of the hIL7 or the functional variant thereof, and/or (ii) RNA encoding an amino acid sequence comprising human IL2 (hIL2), a functional variant thereof, or a functional fragment of the hIL2 or the functional variant thereof. One or both of these RNAs are also designated “immunostimulant RNA” herein. In one embodiment, the immunostimulant, i.e., the hIL, a functional variant thereof, or a functional fragment of the hIL or the functional variant thereof, is fused, either directly or through a linker, to human albumin (hAlb), a functional variant thereof, or a functional fragment of the hAlb or the functional variant thereof.

In one embodiment, the treatment comprises the administration of (iii) RNA, i.e., vaccine RNA, encoding an amino acid sequence, i.e., a vaccine antigen, comprising a target antigen, an immunogenic variant thereof, or an immunogenic fragment of the target antigen or the immunogenic variant thereof, i.e., an antigenic peptide or protein. Thus, the vaccine antigen comprises an epitope of the target antigen for inducing an immune response against the target antigen or cells expressing the target antigen in the subject. RNA encoding vaccine antigen is administered to provide (following expression of the polynucleotide by appropriate target cells) antigen for induction, i.e., stimulation, priming and/or expansion, of an immune response, e.g., antibodies and/or immune effector cells, which is targeted to target antigen or a procession product thereof. In one embodiment, the immune response which is to be induced according to the present disclosure is a B cell-mediated immune response, i.e., an antibody-mediated immune response. Additionally or alternatively, in one embodiment, the immune response which is to be induced according to the present disclosure is a T cell-mediated immune response. In one embodiment, the immune response is an immune response against tumor or cancer cells, in particular tumor or cancer cells expressing a tumor antigen.

The compositions and methods described herein comprise as the active principle single-stranded RNA that may be translated into the respective protein upon entering cells of a recipient. In addition to wildtype or codon-optimized coding sequences, the RNA may contain one or more structural elements optimized for maximal efficacy of the RNA with respect to stability and translational efficiency (5′ cap, 5′ UTR, 3′ UTR, poly(A)-tail). As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These were identified by an ex vivo selection process for sequences that confer RNA stability and augment total protein expression (see WO 2017/060314, herein incorporated by reference). Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency.

Furthermore, in the vaccine RNA, see (secretory signal peptide) and/or MITD (MHC class I trafficking domain) may be fused to the antigen-encoding regions in a way that the respective elements are translated as N- or C-terminal tag, respectively. Fusion-protein tags derived from the sequence encoding the human MHC class I complex (HLA-B51, haplotype A2, B27/B51, Cw2/Cw3), have been shown to improve antigen processing and presentation. Sec may correspond to the 78 bp fragment coding for the secretory signal peptide, which guides translocation of the nascent polypeptide chain into the endoplasmatic reticulum. MITD may correspond to the transmembrane and cytoplasmic domain of the MHC class I molecule, also called MHC class I trafficking domain. Sequences coding for short linker peptides predominantly consisting of the amino acids glycine (G) and serine (S), as commonly used for fusion proteins may be used as GS/Linkers.

The antigen may be administered in combination with helper epitopes to break immunological tolerance. The helper epitopes may be tetanus toxoid-derived, e.g., P2P16 amino acid sequences derived from the tetanus toxoid (TT) of Clostridium tetani. These sequences may support to overcome tolerance mechanisms by providing tumor-unspecific T-cell help during priming. The tetanus toxoid heavy chain includes epitopes that can bind promiscuously to MHC class Il alleles and induce CD4+ memory T cells in almost all tetanus vaccinated individuals. In addition, the combination of TT helper epitopes with tumor-associated antigens is known to improve the immune stimulation compared to the application of tumor-associated antigen alone by providing CD4+ mediated T-cell help during priming. To reduce the risk of stimulating CD8+ T cells, two peptide sequences known to contain promiscuously binding helper epitopes may be used to ensure binding to as many MHC class Il alleles as possible, e.g., P2 and P16.

In one embodiment, a vaccine antigen comprises an amino acid sequence which breaks immunological tolerance. In one embodiment, the amino acid sequence which breaks immunological tolerance comprises helper epitopes, preferably tetanus toxoid-derived helper epitopes. The amino acid sequence which breaks immunological tolerance may be fused to the C-terminus of the vaccine sequence, e.g., antigen sequence, either directly or separated by a linker. Optionally, the amino acid sequence which breaks immunological tolerance may link the vaccine sequence and the MITD.

In one embodiment, the antigen-targeting RNAs are applied together with RNA coding for a helper-epitope to boost the resulting immune response. This RNA coding for a helper-epitope may contain structural elements optimized for maximal efficacy of the RNA with respect to stability and translational efficiency (5′ cap, 5′ UTR, 3′ UTR, poly(A)-tail) described above.

The RNA, i.e., immunostimulant RNA and vaccine RNA, may be formulated in lipid particles to generate serum-stable formulations for intravenous (IV) administration. The immunostimulant RNA may be present in lipid nanoparticles (LNP). RNA-nanoparticles may target liver which results in an efficient expression of the encoded protein. In one embodiment, the immunostimulant RNA described herein is N1-methylpseudouridine modified, dsRNA-purified RNA which is formulated as lipid nanoparticles for intravenous administration. The vaccine RNA may be present in RNA-lipoplexes (LPX). RNA-lipoplexes may target antigen-presenting cells (APCs) in lymphoid organs which results in an efficient stimulation of the immune system. Different RNAs may be separately complexed with lipids to generate particulate formulations. In one embodiment, vaccine RNA is co-formulated as particles with an RNA encoding an amino acid sequence which breaks immunological tolerance.

In one aspect, provided herein is a composition or medical preparation comprising at least one RNA, wherein the at least one RNA encodes:
(i) an amino acid sequence comprising human IL7 (hIL7), a functional variant thereof, or a functional fragment of the hIL7 or the functional variant thereof; and/or
(ii) an amino acid sequence comprising human IL2 (hIL2), a functional variant thereof, or a functional fragment of the hIL2 or the functional variant thereof.

In one embodiment, the amino acid sequence under (i) comprises human albumin (hAlb), a functional variant thereof, or a functional fragment of the hAlb or the functional variant thereof. In one embodiment, the hAlb, the functional variant thereof, or the functional fragment of the hAlb or the functional variant thereof is fused with the hIL7, the functional variant thereof, or the functional fragment of the hIL7 or the functional variant thereof. In one embodiment, the hAlb, the functional variant thereof, or the functional fragment of the hAlb or the functional variant thereof is fused to the C-terminus of the hIL7, the functional variant thereof, or the functional fragment of the hIL7 or the functional variant thereof.

In one embodiment, the amino acid sequence under (ii) comprises human albumin (hAlb), a functional variant thereof, or a functional fragment of the hAlb or the functional variant thereof. In one embodiment, the hAlb, the functional variant thereof, or the functional fragment of the hAlb or the functional variant thereof is fused with the hIL2, the functional variant thereof, or the functional fragment of the hIL2 or the functional variant thereof. In one embodiment, the hAlb, the functional variant thereof, or the functional fragment of the hAlb or the functional variant thereof is fused to the N-terminus of the hIL2, the functional variant thereof, or the functional fragment of the hIL2 or the functional variant thereof.

In one embodiment, each of the amino acid sequences under (i), or (ii) is encoded by a separate RNA.

In one embodiment:
(i) the RNA encoding the amino acid sequence under (i) comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5; and/or
(ii) the amino acid sequence under (i) comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4.

In one embodiment:
(i) the RNA encoding the amino acid sequence under (ii) comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
(ii) the amino acid sequence under (ii) comprises the amino acid sequence of SEQ ID NO: 6, or an amino acid sequence having at least 99%, 98%, 97%, 96%, 95%, 90%, 85%, or 80% identity to the amino acid sequence of SEQ ID NO: 6.

In one embodiment, at least one of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence. In one embodiment, each of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, at least one RNA comprises the 5′ cap m₂^{7,3′-O}Gppp(m₁^{2′-O})ApG. In one embodiment, each RNA comprises the 5′ cap m₂^{7,3′-O}Gppp(m₁^{2′-O})ApG.

In one embodiment, at least one RNA is a modified RNA, in particular a stabilized mRNA. In one embodiment, at least one RNA comprises a modified nucleoside in place of at least one uridine. In one embodiment, at least one RNA comprises a modified nucleoside in place of each uridine. In one embodiment, each RNA comprises a modified nucleoside in place of at least one uridine. In one embodiment, each RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, at least one RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13. In one embodiment, each RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, at least one RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14. In one embodiment, each RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

In one embodiment, at least one RNA comprises a poly-A sequence. In one embodiment, each RNA comprises a poly-A sequence. In one embodiment, the poly-A sequence comprises at least 100 nucleotides. In one embodiment, the poly-A sequence comprises or consists of the nucleotide sequence of SEQ ID NO: 15.

In one embodiment, the amino acid sequence under (i), i.e., the amino acid sequence comprising human IL7 (hIL7), a functional variant thereof, or a functional fragment of the hIL7 or the functional variant thereof, comprises from N-terminus to C-terminus: N-hIL7-GS-linker-hAlb-C.

In one embodiment, the amino acid sequence under (ii), i.e., the amino acid sequence comprising human IL2 (hIL2), a functional variant thereof, or a functional fragment of the hIL2 or the functional variant thereof, comprises from N-terminus to C-terminus: N-hAlb-GS-linker-hIL2-C.

In one embodiment, the RNA is formulated as a liquid, formulated as a solid, or a combination thereof. In one embodiment, the RNA is formulated for injection. In one embodiment, the RNA is formulated for intravenous administration. In one embodiment, the RNA is formulated or is to be formulated as lipid particles. In one embodiment, the RNA lipid particles are lipid nanoparticles (LNP). In one embodiment, the LNP particles comprise 3D-P-DMA, PEG₂₀₀₀-C-DMA, DSPC, and cholesterol.

In one embodiment, the composition or medical preparation is a pharmaceutical composition. In one embodiment, the pharmaceutical composition further comprises one or more pharmaceutically acceptable carriers, diluents and/or excipients.

In one embodiment, the composition or medical preparation is a kit. In one embodiment, the RNA encoding the amino acid sequence under (i) and the RNA encoding the amino acid sequence under (ii) are in separate vials. In one embodiment, the composition or medical preparation comprises instructions for use of the RNAs for treating or preventing cancer.

In one aspect, provided herein is the composition or medical preparation described herein for pharmaceutical use. In one embodiment, the pharmaceutical use comprises a therapeutic or prophylactic treatment of a disease or disorder. In one embodiment, the therapeutic or prophylactic treatment of a disease or disorder comprises treating or preventing cancer.

In one embodiment, the composition or medical preparation is for administration to a human.

In one aspect, provided herein is a method of treating cancer in a subject comprising administering at least one RNA to the subject, wherein the at least one RNA encodes:
(i) an amino acid sequence comprising human IL7 (hIL7), a functional variant thereof, or a functional fragment of the hIL7 or the functional variant thereof; and/or
(ii) an amino acid sequence comprising human IL2 (hIL2), an functional variant thereof, or a functional fragment of the hIL2 or the functional variant thereof.

In one embodiment, the amino acid sequence under (i) comprises human albumin (hAlb), a functional variant thereof, or a functional fragment of the hAlb or the functional variant thereof. In one embodiment, the hAlb, the functional variant thereof, or the functional fragment of the hAlb or the functional variant thereof is fused with the hIL7, the functional variant thereof, or the functional fragment of the hIL7 or the functional variant thereof. In one embodiment, the hAlb, the functional variant thereof, or the functional fragment of the hAlb or the functional variant thereof is fused to the C-terminus of the hIL7, the functional variant thereof, or the functional fragment of the hIL7 or the functional variant thereof.

In one embodiment, the amino acid sequence under (ii) comprises human albumin (hAlb), a functional variant thereof, or a functional fragment of the hAlb or the functional variant thereof. In one embodiment, the hAlb, the functional variant thereof, or the functional fragment of the hAlb or the functional variant thereof is fused with the hIL2, the functional variant thereof, or the functional fragment of the hIL2 or the functional variant thereof. In one embodiment, the hAlb, the functional variant thereof, or the functional fragment of the hAlb or the functional variant thereof is fused to the N-terminus of the hIL2, the functional variant thereof, or the functional fragment of the hIL2 or the functional variant thereof.

In one embodiment, each of the amino acid sequences under (i), or (ii) is encoded by a separate RNA.

In one embodiment:
(i) the RNA encoding the amino acid sequence under (i) comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5; and/or
(ii) the amino acid sequence under (i) comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4.

In one embodiment:
(i) the RNA encoding the amino acid sequence under (ii) comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
(ii) the amino acid sequence under (ii) comprises the amino acid sequence of SEQ ID NO: 6, or an amino acid sequence having at least 99%, 98%, 97%, 96%, 95%, 90%, 85%, or 80% identity to the amino acid sequence of SEQ ID NO: 6.

In one embodiment, at least one of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence. In one embodiment, each of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, at least one RNA is a modified RNA, in particular a stabilized mRNA. In one embodiment, at least one RNA comprises a modified nucleoside in place of at least one uridine. In one embodiment, at least one RNA comprises a modified nucleoside in place of each uridine. In one embodiment, each RNA comprises a modified nucleoside in place of at least one uridine. In one embodiment, each RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, at least one RNA comprises the 5′ cap m₂^{7,3′-O}Gppp(m₁^{2′-O})ApG. In one embodiment, each RNA comprises the 5′ cap m₂^{7,3′-O}Gppp(m₁^{2′-O})ApG.

In one embodiment, at least one RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13. In one embodiment, each RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, at least one RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14. In one embodiment, each RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

In one embodiment, at least one RNA comprises a poly-A sequence. In one embodiment, each RNA comprises a poly-A sequence. In one embodiment, the poly-A sequence comprises at least 100 nucleotides. In one embodiment, the poly-A sequence comprises or consists of the nucleotide sequence of SEQ ID NO: 15.

In one embodiment, the amino acid sequence under (i), i.e., the amino acid sequence comprising human IL7 (hIL7), a functional variant thereof, or a functional fragment of the hIL7 or the functional variant thereof, comprises from N-terminus to C-terminus: N-hIL7-GS-linker-hAlb-C.

In one embodiment, the amino acid sequence under (ii), i.e., the amino acid sequence comprising human IL2 (hIL2), a functional variant thereof, or a functional fragment of the hIL2 or the functional variant thereof, comprises from N-terminus to C-terminus: N-hAlb-GS-linker-hIL2-C.

In one embodiment, the RNA is formulated as a liquid, formulated as a solid, or a combination thereof. In one embodiment, the RNA is administered by injection. In one embodiment, the RNA is administered by intravenous administration. In one embodiment, the RNA is formulated as lipid particles. In one embodiment, the RNA lipid particles are lipid nanoparticles (LNP). In one embodiment, the LNP particles comprise 3D-P-DMA, PEG₂₀₀₀-C-DMA, DSPC, and cholesterol. In one embodiment, the RNA is formulated as a pharmaceutical composition. In one embodiment, the pharmaceutical composition further comprises one or more pharmaceutically acceptable carriers, diluents and/or excipients.

In one embodiment, the subject is a human.

In one embodiment, the composition or medical preparation described herein comprises RNA, which encodes:
(iii) an amino acid sequence comprising a target antigen, an immunogenic variant thereof, or an immunogenic fragment of the target antigen or the immunogenic variant thereof.

In one embodiment, the method described herein comprises administering RNA to the subject, wherein the RNA encodes:
(iii) an amino acid sequence comprising a target antigen, an immunogenic variant thereof, or an immunogenic fragment of the target antigen or the immunogenic variant thereof.

In one embodiment, the target antigen is a tumor antigen.

In one embodiment, the amino acid sequence under (iii) comprises an amino acid sequence enhancing antigen processing and/or presentation.

In one embodiment, the amino acid sequence enhancing antigen processing and/or presentation comprises an amino acid sequence corresponding to the transmembrane and cytoplasmic domain of a MHC molecule, preferably a MHC class I molecule.

In one embodiment, the amino acid sequence enhancing antigen processing and/or presentation comprises the amino acid sequence of SEQ ID NO: 9, or an amino acid sequence having at least 99%, 98%, 97%, 96%, 95%, 90%, 85%, or 80% identity to the amino acid sequence of SEQ ID NO: 9.

In one embodiment, the amino acid sequence enhancing antigen processing and/or presentation further comprises an amino acid sequence coding for a secretory signal peptide.

In one embodiment, the secretory signal peptide comprises the amino acid sequence of SEQ ID NO: 8, or an amino acid sequence having at least 99%, 98%, 97%, 96%, 95%, 90%, 85%, or 80% identity to the amino acid sequence of SEQ ID NO: 8.

In one embodiment, the amino acid sequence under (iii) comprises an amino acid sequence which breaks immunological tolerance and/or the RNA is co-administered with RNA encoding an amino acid sequence which breaks immunological tolerance.

In one embodiment, the amino acid sequence which breaks immunological tolerance comprises helper epitopes, preferably tetanus toxoid-derived helper epitopes.

In one embodiment, the amino acid sequence which breaks immunological tolerance comprises the amino acid sequence of SEQ ID NO: 10, or an amino acid sequence having at least 99%, 98%, 97%, 96%, 95%, 90%, 85%, or 80% identity to the amino acid sequence of SEQ ID NO: 10.

In one embodiment the amino acid sequence under (iii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, the RNA is a modified RNA, in particular a stabilized mRNA. In one embodiment, the RNA comprises a modified nucleoside in place of at least one uridine. In one embodiment, the RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, the RNA comprises the 5′ cap m₂^{7,2′-O}Gppₛp(5′)G. In one embodiment, the RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In one embodiment, the RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

In one embodiment, the RNA comprises a poly-A sequence.

In one embodiment, the poly-A sequence comprises at least 100 nucleotides.

In one embodiment, the poly-A sequence comprises or consists of the nucleotide sequence of SEQ ID NO: 15.

In one embodiment, the amino acid sequence under (iii), i.e., the amino acid sequence comprising a target antigen, an immunogenic variant thereof, or an immunogenic fragment of the target antigen or the immunogenic variant thereof, comprises from N-terminus to C-terminus: N-antigen-amino acid sequence which breaks immunological tolerance-amino acid sequence enhancing antigen processing and/or presentation-C.

In one embodiment, the RNA is formulated as a liquid, formulated as a solid, or a combination thereof.

In one embodiment, the RNA is formulated for injection and/or is administered by injection.

In one embodiment, the RNA is formulated for intravenous administration and/or is administered by intravenous administration.

In one embodiment, the RNA is formulated or is to be formulated as lipoplex particles.

In one embodiment, the RNA lipoplex particles are obtainable by mixing the RNA with liposomes.

In one aspect, provided herein is RNA described herein, e.g.,
(i) RNA encoding an amino acid sequence comprising human IL7 (hIL7), a functional variant thereof, or a functional fragment of the hIL7 or the functional variant thereof; and/or
(ii) RNA encoding an amino acid sequence comprising human IL2 (hIL2), a functional variant thereof, or a functional fragment of the hIL2 or the functional variant thereof; and optionally
(iii) RNA encoding an amino acid sequence comprising a target antigen, an immunogenic variant thereof, or an immunogenic fragment of the target antigen or the immunogenic variant thereof, for use in a method described herein.

In the above and further aspects, provided herein is a composition comprising lipid nanoparticles (LNP) comprising RNA, 3D-P-DMA, a pegylated lipid, a neutral lipid, in particular a phospholipid, and a steroid such as cholesterol. In one embodiment, the pegylated lipid is PEG₂₀₀₀-C-DMA. In one embodiment, the phospholipid is DSPC. In one embodiment, the pegylated lipid is PEG₂₀₀₀-C-DMA and the phospholipid is DSPC. In some embodiments, the 3D-P-DMA is present in the LNP in an amount from about 40 to about 60 mole percent, the pegylated lipid such as PEG₂₀₀₀-C-DMA is present in the LNP in an amount from about 1 to about 10 mole percent, the neutral lipid such as DSPC is present in the LNP in an amount from about 5 to about 15 mole percent, and the steroid such as cholesterol is present in the LNP in an amount from about 30 to about 50 mole percent. In some embodiments, the 3D-P-DMA is present in the LNP in an amount of about 54 mole percent, the pegylated lipid such as PEG₂₀₀₀-C-DMA is present in the LNP in an amount of about 1.6 mole percent, the neutral lipid such as DSPC is present in the LNP in an amount of about 11 mole percent, and the steroid such as cholesterol is present in the LNP in an amount of about 33 mole percent.

In one embodiment, the composition is an aqueous composition. In one embodiment, the composition comprises a Tris/HCl buffer. In one embodiment, the composition comprises sucrose and/or maltose. In one embodiment, the RNA is (i) RNA encoding an amino acid sequence comprising human IL7 (hIL7), a functional variant thereof, or a functional fragment of the hIL7 or the functional variant thereof; and/or (ii) RNA encoding an amino acid sequence comprising human IL2 (hIL2), a functional variant thereof, or a functional fragment of the hIL2 or the functional variant thereof. Embodiments of this RNA are described herein.

### BRIEF DESCRIPTION OF THE DRAWINGS

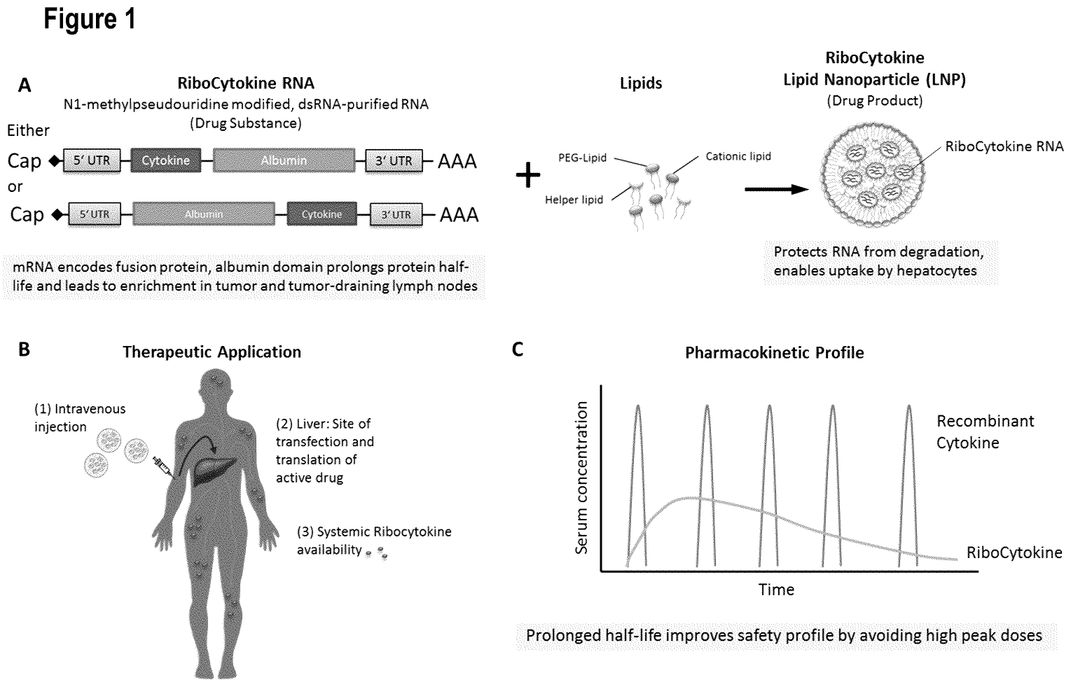

FIG. 1: Concept of the RiboCytokine® platform technology
(A) Cytokines fused to serum albumin are encoded by N1-methylpseudouridine modified single-stranded RNA (RiboCytokine RNA). The RNA is formulated as LNPs to form the RiboCytokine product. (B) Systemically injected LNPs are internalized and the encapsulated RNA is translated by liver cells, yielding high systemic amounts of the biologically active RiboCytokine drug. (C) Fusion of the RiboCytokine drug to serum albumin confers prolonged bioavailability and protracted clearance.
dsRNA=double-stranded RNA, LNP=lipid nanoparticle, RNA=ribonucleic acid, UTR=untranslated region.

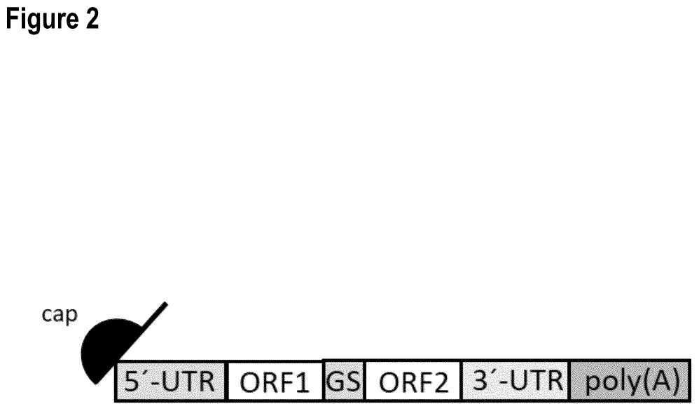

FIG. 2: Schematic illustration of the general structure of the cytokine-encoding mRNA with 5′-cap, 5′- and 3′-UTRs, coding sequence (ORF1 and ORF2), GS-Linker between ORFs (GS) and poly(A)-tail
mRNA=messenger ribonucleic acid, ORF=open reading frame, UTR=untranslated region.

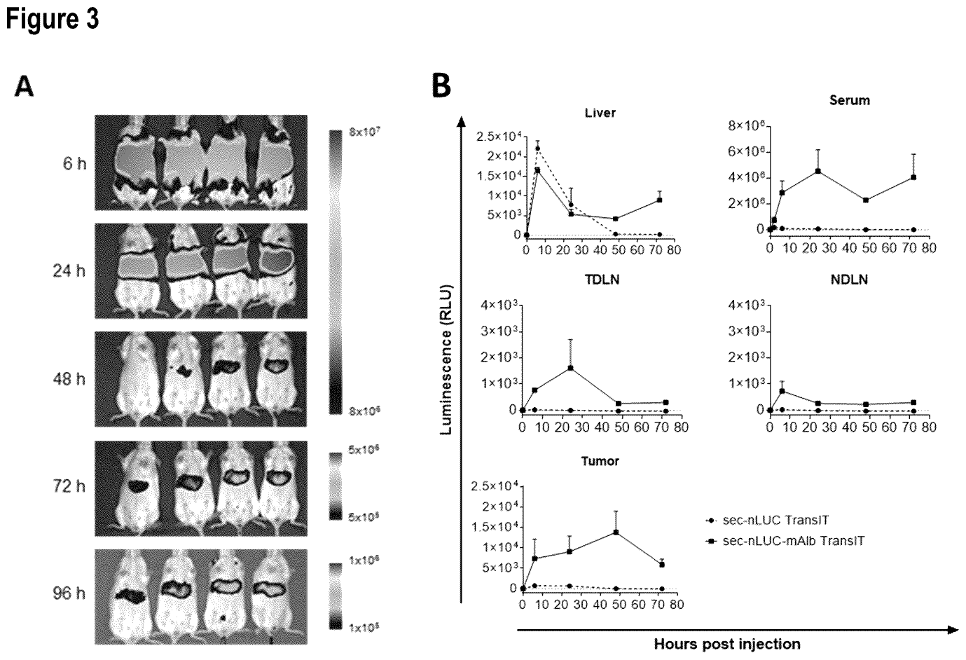

FIG. 3: Liver-targeted translation of LNP-formulated RNA and biodistribution of secreted albumin-fusion protein
(A) Liver-specific translation of LUC in BALB/c mice treated IV with 3 μg LUC RNA formulated as LNPs. The cumulative intensity of emitted photons in live animals originating from LUC-expressing cells is represented in pseudo color according to the scale bars (blue=low; red=high). (B) Extended systemic persistence and increased bioavailability of a secreted form of nano-LUC (sec-nLUC) fused to mouse albumin (sec-nLUC-mAlb) in the tumor and tumor-draining lymph nodes of CT26 tumor-bearing BALB/c mice after injection of 3 μg lipid/polymer (TransIT)-formulated RNA. Bioluminescence intensity was quantified from 50 μL serum or 30 μg total protein derived from tissue lysates obtained from cryoconserved tissues by Nano-Glo′ luciferase assay. Data are shown as mean±standard error of the mean (n=3 mice per group and time point).
h=hour, LNP=lipid nanoparticle, LUC=luciferase, mAlb=murine albumin, NDLN=non-draining lymph node, RLU=relative light unit, TDLN=tumor-draining lymph node.

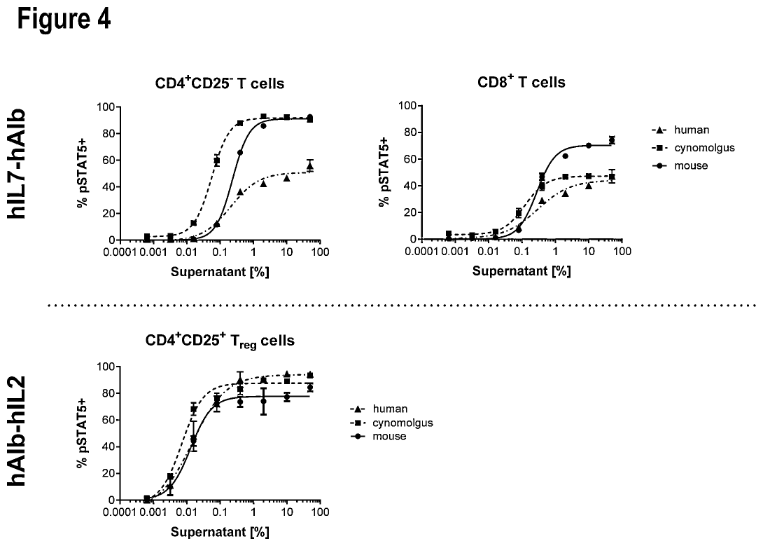

FIG. 4: hIL7-hAlb and hAlb-hIL2 display similar activity on human, cynomolgus monkey and mouse immune cells
Biological activity of hIL7-hAlb and hAlb-hIL2 was tested in a STAT5 phosphorylation bioassay using human, mouse and cynomolgus monkey PBMCs. PBMCs were incubated with serial dilutions of hIL7-hAlb or hAlb-hIL2-containing supernatants generated by lipofection of HEK293T/17 cells with the respective RNA construct. Phosphorylation of STAT5 was analyzed in previously identified most responsive indicator immune cell subsets per cytokine via flow cytometry. The % pSTAT5-positive fraction of CD4⁺CD25⁻ and CD8⁺ T cells for hIL7-hAlb, and CD4⁺CD25⁺ T_{regs} for hAlb-hIL2 is plotted as a function of the supernatant dilution. Data shown are mean±SD of n=2 technical replicates fitted with a 4-parameter logarithmic fit to calculate EC50 values as an objective measure of bioactivity. Both hIL7-hAlb and hAlb-hIL2 are functional in all three species tested, hence identifying mouse and cynomolgus monkey as relevant species for in vivo pharmacology assessment.

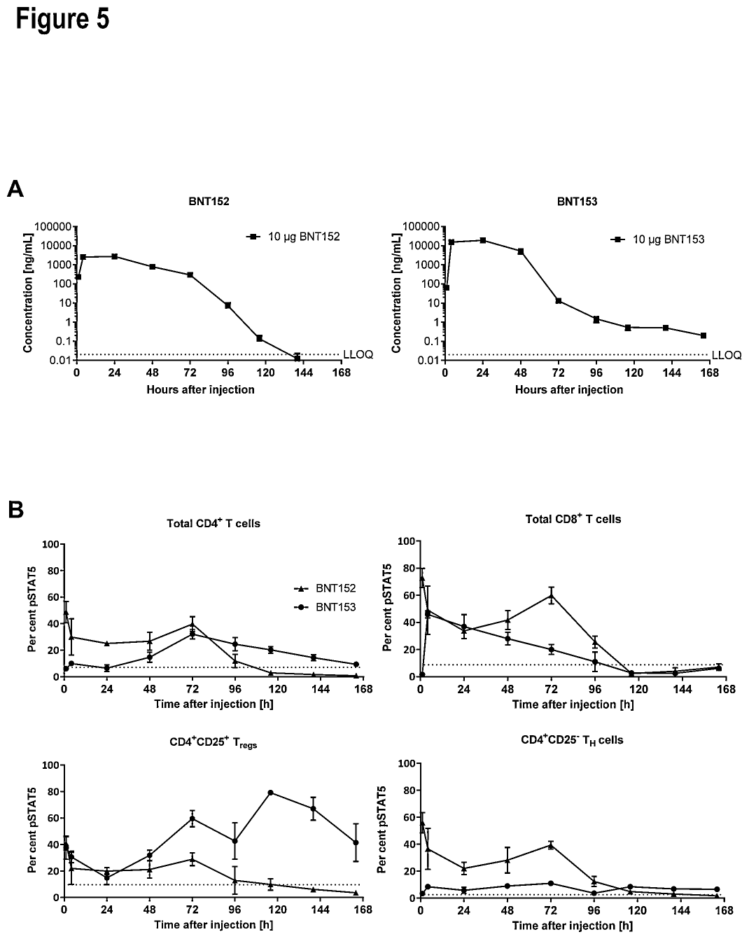

FIG. 5: In vivo activity of BNT152 (hIL7-hAlb) and BNT153 (hAlb-hIL2) on T cell subsets in mouse blood assessed via STAT5 phosphorylation
BALB/c mice (n=3 per group and time point) were injected IV with 10 μg BNT152 or BNT153. Control animals received 10 μg hAlb RNA formulated as LNP. Blood was drawn 1, 4, 24, 48, 72, 96, 116, 140 and 164 h after injection and phosphorylation of STAT5 was analyzed in total CD4⁺ T cells, CD4⁺CD25⁺ T_{regs}, CD4⁺CD25⁻ T_{H} cells and CD8⁺ T cells by flow cytometry. Data received from the control group at 1 h after injection served as baseline and is indicated by horizontal dotted lines. Data are presented as mean±standard error of the mean. BNT152-translated hIL7-hAlb activated total CD4⁺ T cells, CD4⁺CD25-T_{H} cells and total CD8⁺ T cells. While BNT153-translated hAlb-hIL2 only initially stimulated phosphorylation of STAT5 in CD8⁺ T cells and hardly affected signaling in CD4⁺CD25 T_{H} cells, CD4⁺CD25⁺ T_{regs} profited from enhanced hAlb-hIL2 availability.

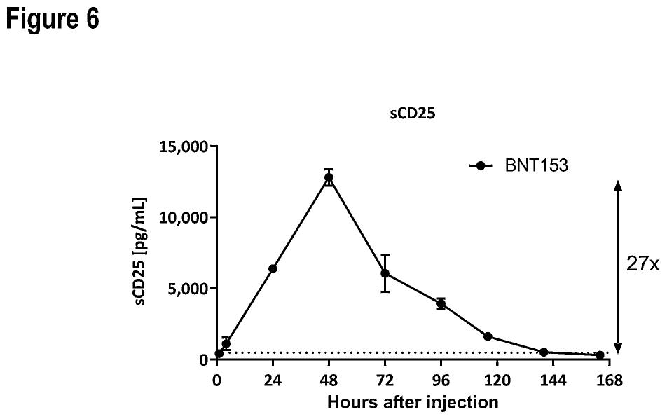

FIG. 6: BNT153 treatment induces secretion of soluble CD25 in mice
BALB/c mice were injected IV with 10 μg BNT153 (encoding hAlb-hIL2) or hAlb (control) LNP-formulated RNA. Blood was drawn at 1, 4, 24, 48, 72, 96, 116, 140 and 164 h after injection. Soluble CD25 levels in serum were determined using the mouse CD25/IL-2Ra DuoSet ELISA kit. Data shown are mean±SD of n=3 mice per group and time point. Dotted line represents baseline sCD25 levels in hAlb-treated animals. hAlb-hIL2 exposure after treatment with BNT153 led to elevated secretion of sCD25. The sCD25 Cₘₐₓ level in the serum was >27-fold higher in hAlb-hIL2-versus hAlb-exposed animals.

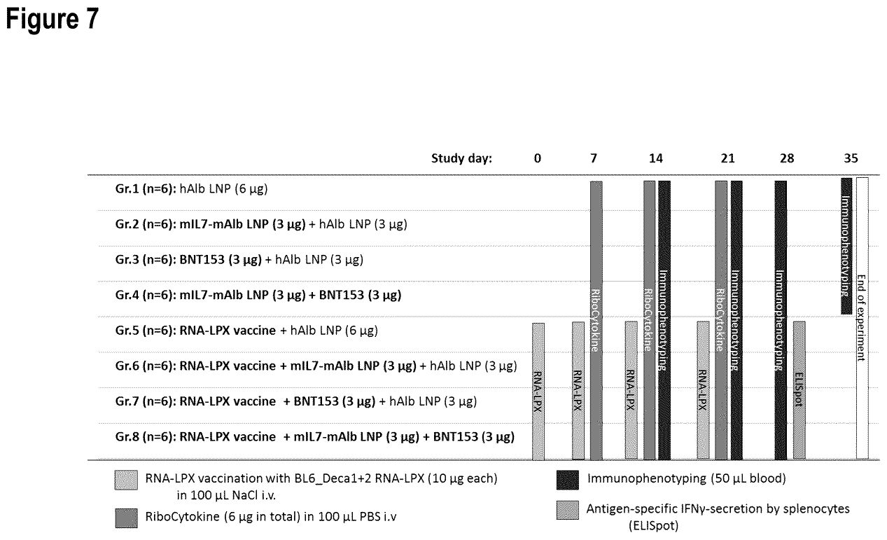

FIG. 7: Study design: Bioactivity of mIL7-mAlb LNP and BNT153 on immune cell subsets in mice
Groups 2-4 were treated with RNA-LNP encoding mouse surrogate IL7 (mIL7) fused to mouse serum albumin (mAlb), mIL7-mAlb LNP, BNT153 or the combination on Day 7, 14 and 21. Group 1 was treated with LNP-formulated hAlb as control. Groups 5-8 were additionally vaccinated with an RNA-LPX vaccine encoding a total of 20 tumor antigens on two “decatope” RNAs (BL6_Deca1+2) on Days 0, 7, 14 and 21. Immunophenotyping was performed on Days 14, 21, 28 and 35.

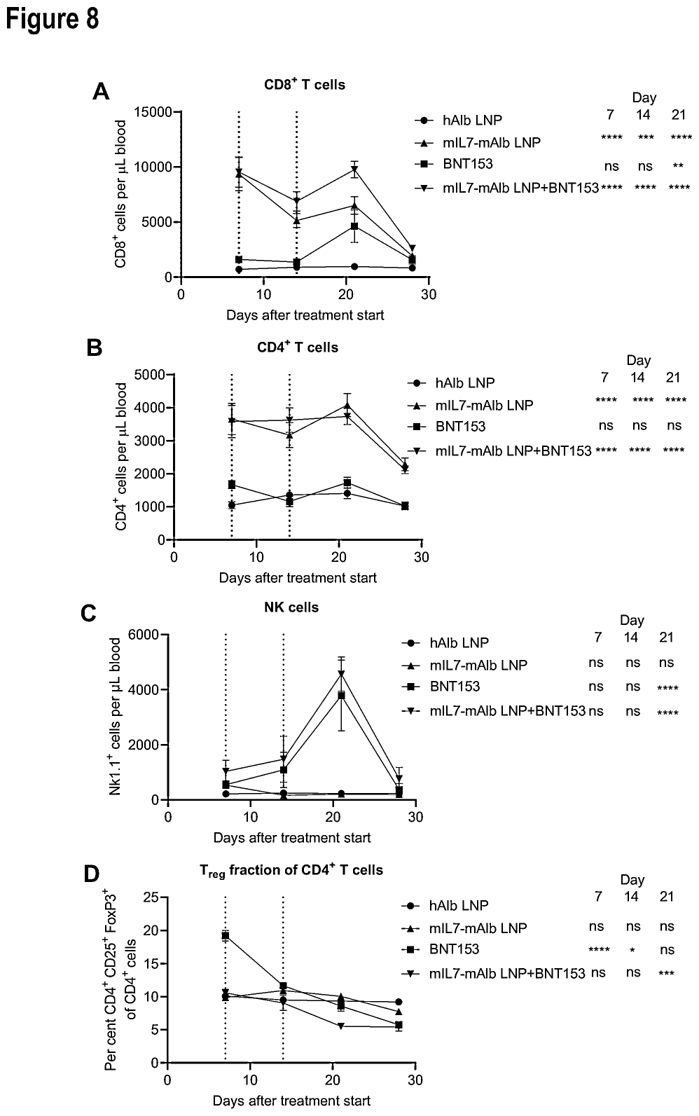

FIG. 8: Quantification of immune cell subsets in the blood upon treatment with mIL7-mAlb LNP, BNT153, or a combination thereof
Mice were treated with either control RiboCytokine (hAlb), mIL7-mAlb LNP or BNT153 as illustrated in FIG. 7 (Groups 1 to 4). (A) CD8⁺ T cell, (B) CD4⁺ T cell, and (C) NK cell numbers per L blood as well as (D) fraction of FoxP3⁺CD25⁺CD4⁺ T_{regs} in the blood upon RiboCytokine treatment quantified by flow cytometry. Treatment days are indicated by vertical dotted lines. ns=not significant; * p≤0.05, ** p≤0.01, *** p≤0.001, **** p≤0.0001. mIL7-mAlb LNP significantly increased CD4⁺ and CD8⁺ T cell numbers. BNT153 increased both CD8⁺ T cell and NK cell numbers as well as the fraction of T_{regs} among CD4⁺ T cells. Combination treatment resulted in an elevation of all three effector populations while the Treg fraction remained at or below baseline levels.

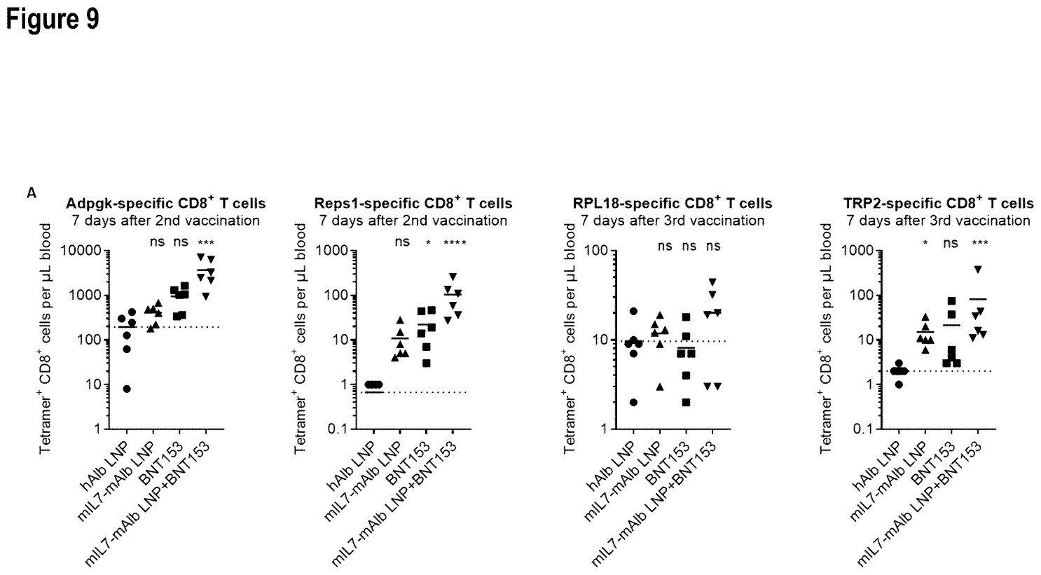

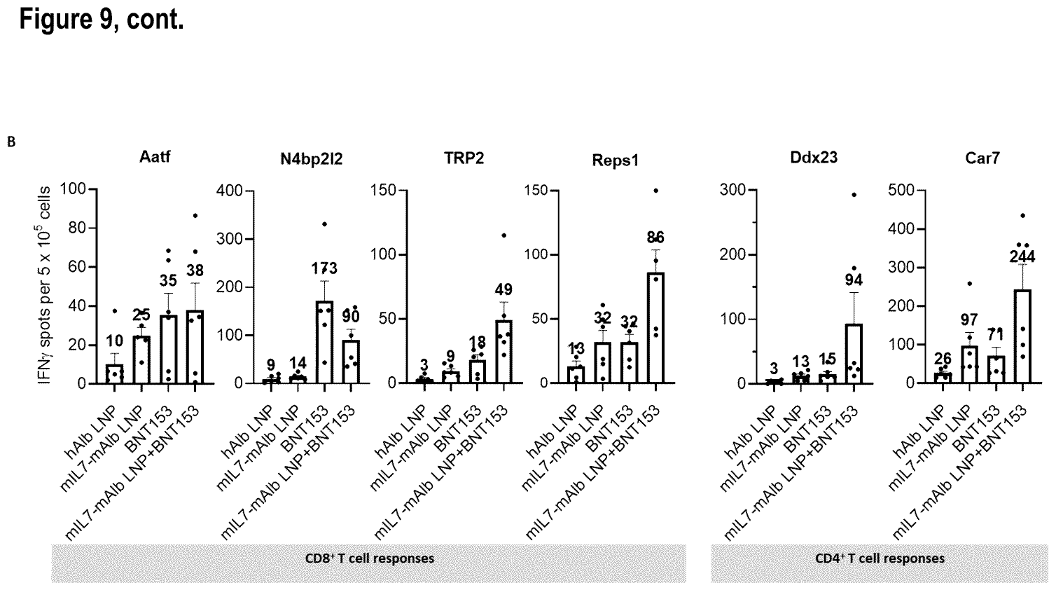

FIG. 9: Quantification of antigen-specific T cells in the blood and spleen of mIL7-mAlb LNP and BNT153 treated, RNA-LPX vaccinated mice
C57BL/6 mice were treated with either control RiboCytokine (hAlb), mIL7-mAlb LNP, BNT153 or mIL7-mAlb LNP plus BNT153 in combination with RNA-LPX vaccination against 20 tumor antigens as illustrated in FIG. 7 (Groups 5-8). (A) Quantification of tumor antigen-specific CD8⁺ T cell responses in the blood by flow cytometry. (B) Number of IFNγ spots per 5×10⁵ splenocytes as measured by ELISpot assay. Splenocytes were stimulated ex vivo with single peptides specific for selected vaccine targets. TRP2 is a self-antigen, all other responses target mutated neoantigens. ns=not significant; * p≤0.05, ** p≤0.01, *** p≤0.001, **** p≤0.0001. mIL7-mAlb LNP and BNT153 treatment increased the number of vaccine-induced tumor antigen-specific CD8⁺ T cells in the blood as well as the number of IFNγ-secreting CD4⁺ and CD8⁺ T cells in the spleen. For the majority of T cell antigens, highest responses were observed in the mIL7-mAlb LNP plus BNT153 combination group.

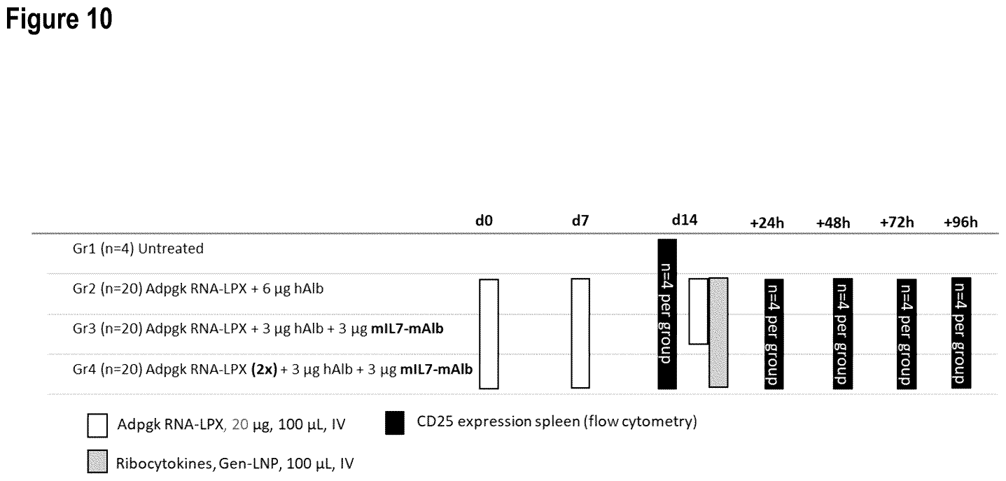

FIG. 10: Study design: CD25 expression on antigen-specific CD8⁺ T cells
C57BL/6 mice were immunized twice with an RNA-LPX vaccine encoding the neo-antigen Adpgk on Day 0 and 7 (groups 2-4). On Day 14, groups 2 and 3 were treated with mIL7-mAlb LNP or 3 μg hAlb LNP in addition to the RNA-LPX vaccine, or with mIL7-mAlb alone (group 4). Animals that received no treatment served to assess CD25 baseline expression on Day 14 (group 1). T cell subsets in the spleen were analyzed by flow cytometry 24, 48, 72 and 96 h after treatment on Day 14.

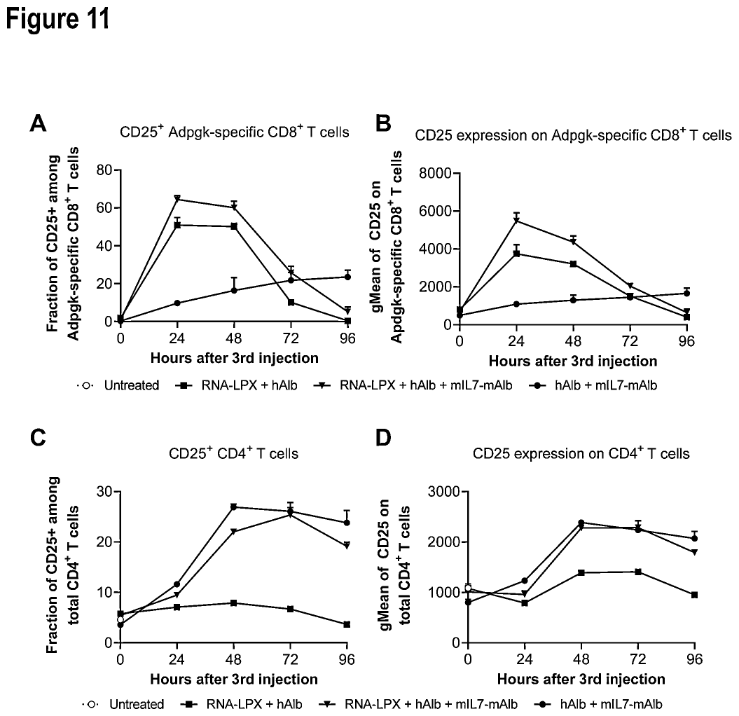

FIG. 11: mIL7-mAlb LNP enhances CD25 expression on antigen-specific CD8⁺ T cells
Mice were treated as illustrated in FIG. 10. Fraction of CD25⁺ among (A) antigen-specific CD8⁺ T cells, and (C) CD4⁺ T cells. CD25 expression on (B) antigen-specific CD8⁺ T cells, and (D) CD4⁺ T cells. Treatment with mIL7-mAlb LNP substantially increased the fraction of CD25⁺ cells among antigen-specific CD8⁺ and CD4⁺ T cells as well as their CD25 expression.

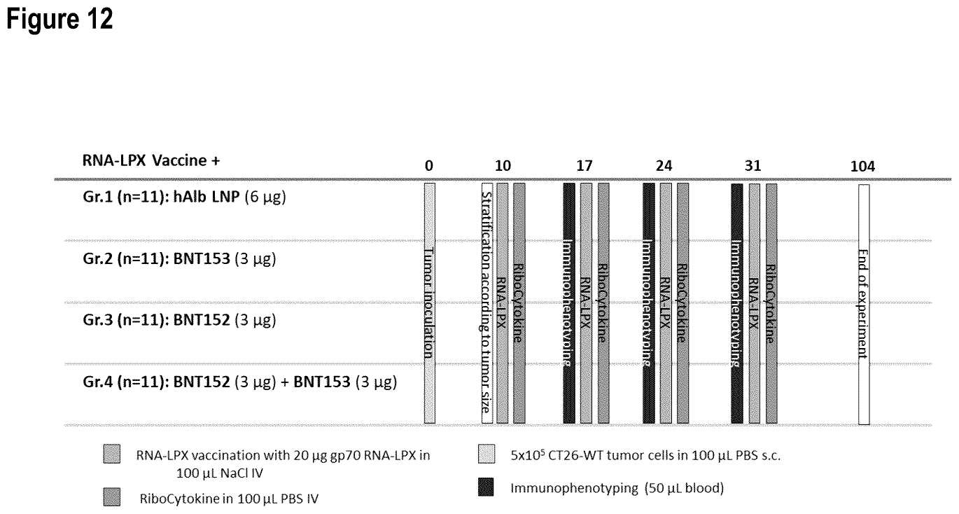

FIG. 12: Study design: Anti-tumor activity of BNT152, BNT153 and the combination together with RNA-LPX vaccination in the CT26 mouse colon carcinoma model
All groups were inoculated s.c. with CT26 tumor cells on Day 0. Groups 2-4 were treated with BNT152, BNT153 or the combination on Days 10, 17, 24 and 31. Group 1 was treated with LNP-formulated RNA encoding hAlb (hAlb-LNP) as control. All groups were additionally vaccinated with an RNA LPX vaccine encoding the tumor-specific antigen gp70. Immunophenotyping was performed on Days 17, 24, and 31.

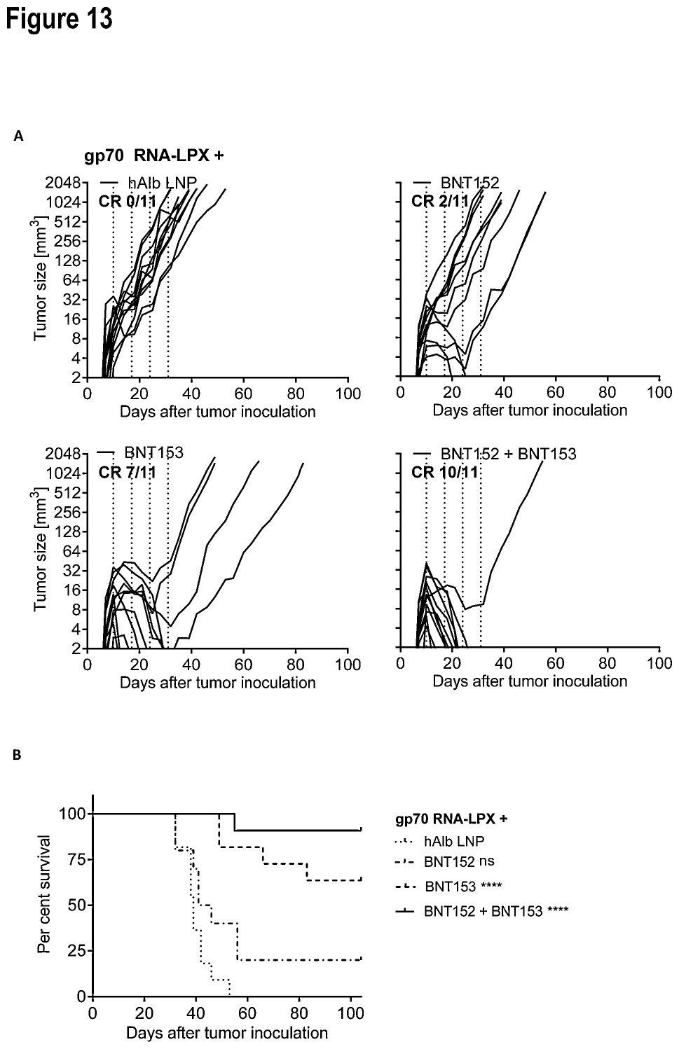

FIG. 13: Tumor growth and survival after treatment with BNT152, BNT153 or both in combination with RNA-LPX vaccination in the CT26 mouse colon carcinoma model
(A) Individual tumor growth and (B) survival of mice treated with BNT152, BNT153 or the combination of BNT152 plus BNT153 together with gp70 RNA-LPX vaccination. Mice were sacrificed once termination criteria, such as a tumor size >1500 m³, were reached. Treatment days are indicated by vertical dotted lines.
CR=complete response; ns=not significant; * p≤0.05, ** p≤ 0.01, *** p≤0.001, **** p≤0.0001. Combination of BNT152 plus BNT153 revealed superior anti-tumor potency together with RNA-LPX vaccination as compared to either RiboCytokine alone.

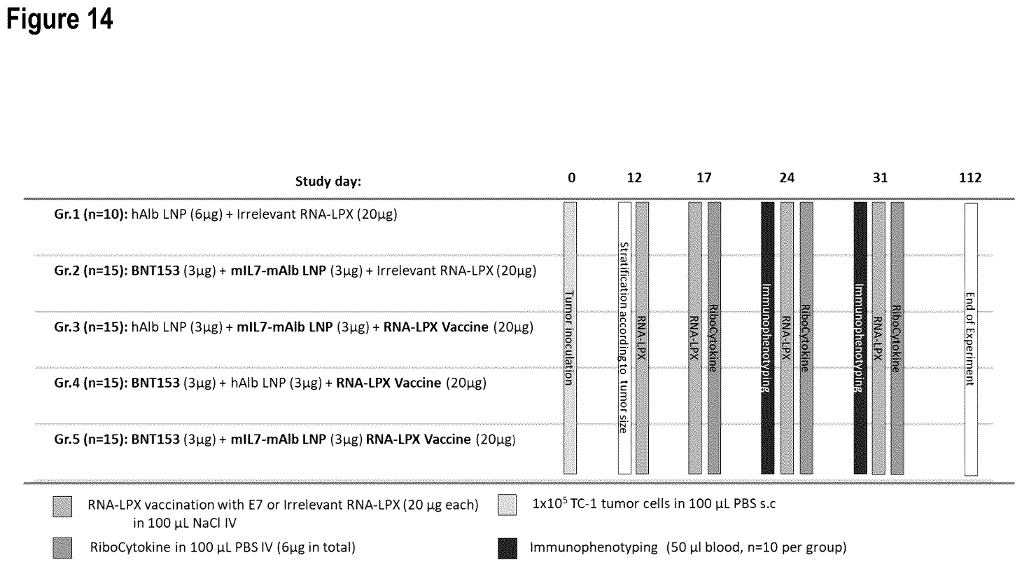

FIG. 14: Study design: Anti-tumor activity and effects on the immune cell compartment of mIL7-mAlb LNP, BNT153 and the combination, together with RNA-LPX vaccination, in the TC-1 mouse lung carcinoma model
All groups were inoculated s.c. with TC-1 tumor cells. Groups 2-5 were treated with LNP-formulated RNA encoding mIL7-mAlb LNP, BNT153 or the combination, together with an RNA-LPX vaccine encoding the tumor-specific antigen E7. Group 1 was treated with LNP-formulated RNA encoding hAlb (hAlb LNP) and irrelevant, non-antigen coding RNA-LPX as control.

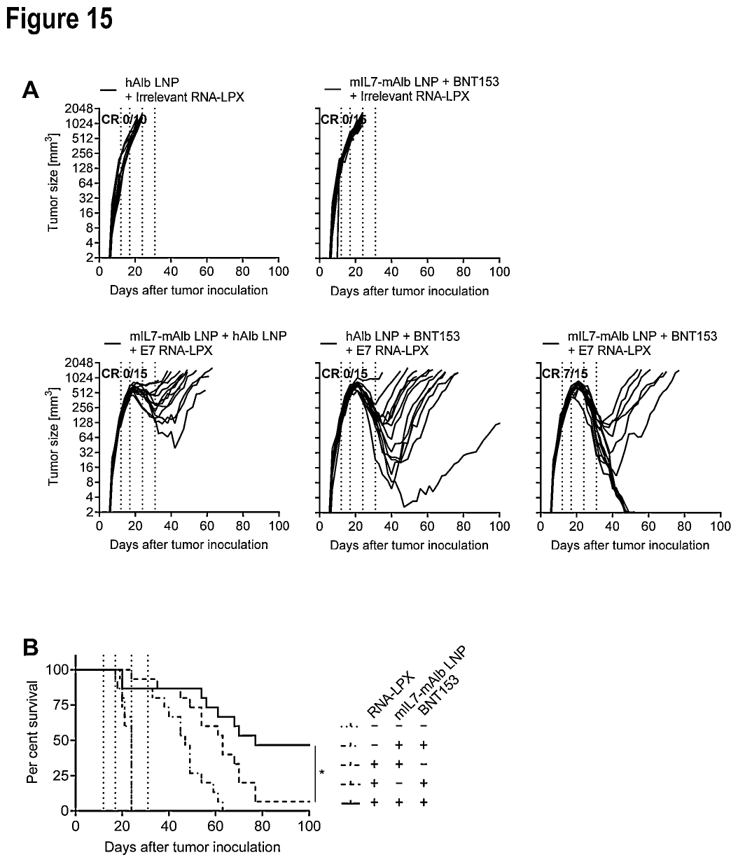

FIG. 15: Tumor growth and survival after treatment with mIL7-mAlb LNP, BNT153 and the combination, together with RNA-LPX vaccination, in the TC-1 lung carcinoma model
Mice were treated either with LNP-formulated RNA encoding hAlb, mIL7-mAlb LNP, BNT153 or the combination of mIL7-mAlb LNP plus BNT153 together with RNA-LPX vaccination encoding the viral tumor antigen E7 or irrelevant RNA-LPX control as illustrated in FIG. 14. (A) Individual tumor growth and (B) survival. CR, complete response. Mice were sacrificed once termination criteria, such as a tumor size >1500 m³, were reached. Treatment days are indicated by vertical dotted lines. ns=not significant; *p≤0.05, ** p≤0.01, *** p≤0.001, **** p≤0.0001. With TC-1 being a weakly immunogenic (‘cold’) tumor without the presence of a pre-existing T cell response, RiboCytokine treatment was not effective without RNA-LPX vaccination. When combined with RNA-LPX vaccination, RiboCytokine treatment resulted in potent tumor control. Only when both mIL7-mAlb LNP plus BNT153 were combined with RNA-LPX vaccination, a substantial fraction of mice (7/15) rejected their tumors.

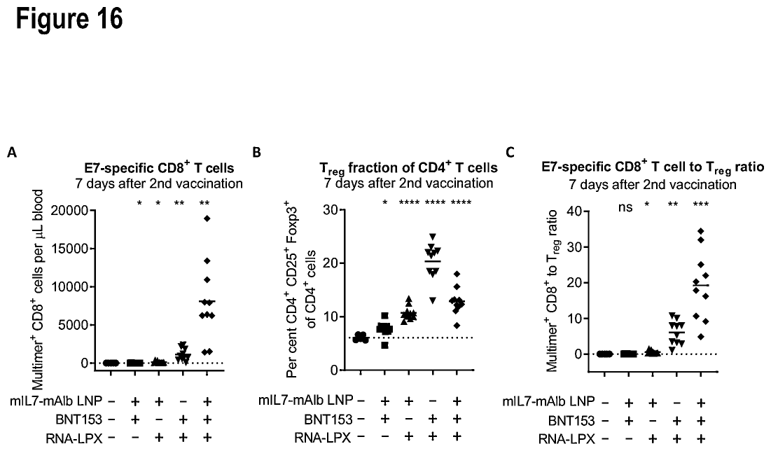

FIG. 16: Quantification of immune cell subsets in the blood upon treatment with mIL7-mAlb LNP, BNT153 and the combination, together with RNA-LPX vaccination, in the TC-1 lung carcinoma model
Mice were treated either with LNP-formulated RNA encoding hAlb, mIL7-mAlb LNP, BNT153 or the combination of mIL7-mAlb LNP plus BNT153 together with RNA-LPX vaccination encoding the viral tumor antigen E7 or irrelevant RNA-LPX control as illustrated in FIG. 14. (A) E7-specific CD8⁺ T cell numbers, (B) Treg fraction among CD4⁺ T cells as well as (C) ratio between E7-specific T cell and Treg numbers. ns=not significant; * p≤0.05, ** p<0.01, *** p≤0.001, **** p≤0.0001. Combination of mIL7-mAlb LNP plus BNT153 strongly boosts RNA-LPX vaccine-induced E7 tumor antigen-specific CD8⁺ T cells. mIL7-mAlb LNP alleviates BNT153-mediated increase of T_{regs}, resulting in a significant increase of the E7-specific CD8⁺ T cell to Treg ratio.

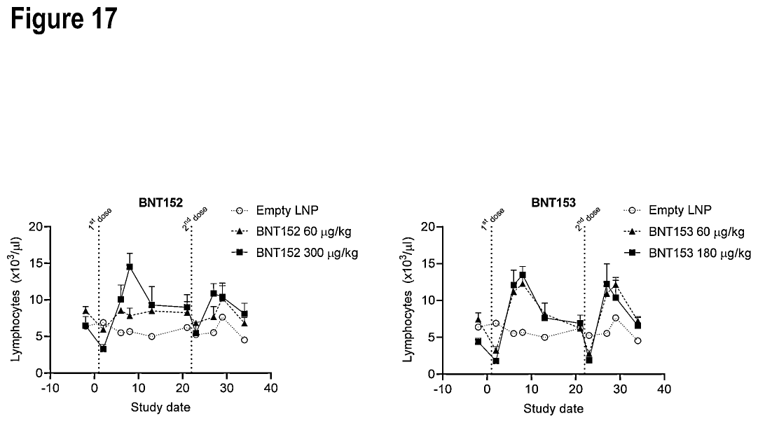

FIG. 17: Lymphocyte counts in the blood of cynomolgus monkeys after BNT152 or BNT153 administration
Cynomolgus monkeys (n=3 per group) were injected IV with 60 and 300 μg/kg BNT152 or 60 and 180 μg/kg BNT153 on Days 1 and 22. Control animals were treated with empty LNPs (lipid dose adapted to 120 μg RNA/kg). Blood was drawn and hematology parameters were analyzed at pre-dose as well as on Days 2, 6, 8, 13, 21, 23, 27, 29, and 34. Mean values of absolute lymphocyte counts are presented. Error bars represent standard error of the mean. Vertical dotted lines indicate days of administration. Treatment with BNT152 and BNT153 transiently decreased the lymphocyte counts in all groups, followed by a strong transient lymphoproliferation in the 60 μg/kg and 180 μg/kg BNT153- and 300 μg/kg BNT152-treated groups.

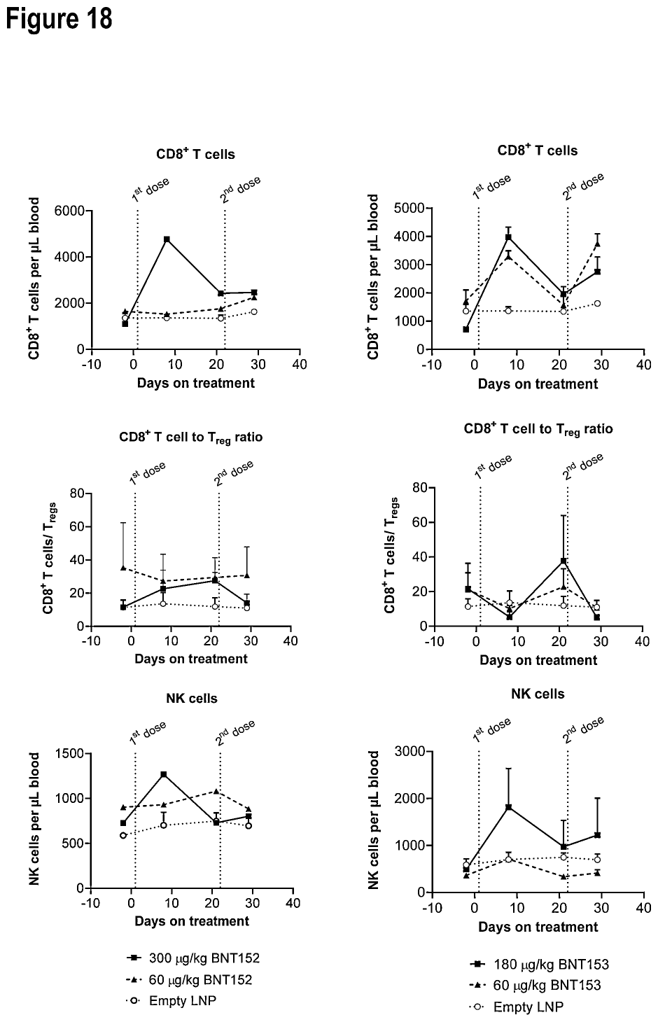

FIG. 18: T cell subsets and NK cells in the blood of cynomolgus monkeys injected with BNT152 or BNT153
Cynomolgus monkeys (n=3 per group) were injected IV with 60 or 300 μg/kg BNT152 or 60 or 180 μg/kg BNT153 on Days 1 and 22. Control animals were treated with empty LNPs (lipid dose adapted to 120 μg RNA/kg). Blood was drawn for flow cytometry analysis of T cell subsets and NK cells at pre-dose, Day 8, 21 and 29. Mean values of absolute CD8⁺ T cell and NK cell numbers, as well as the CD8⁺ T cell to Treg ratios are presented. Error bars represent standard error of the mean. Vertical dotted lines indicate days of administration. Treatment with 300 μg/kg BNT152 and 60 μg/kg or 180 g/kg BNT153 transiently increased CD8⁺ T cell and NK cell numbers. BNT153 treatment transiently decreased the CD8⁺ T cell to Treg ratio in both tested dose groups.

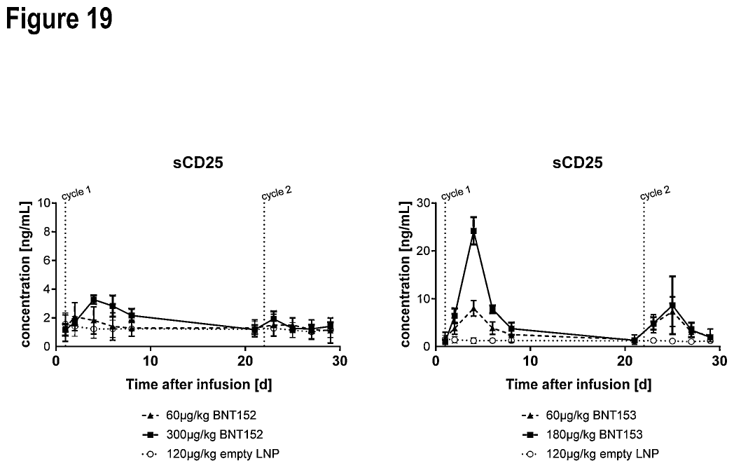

FIG. 19: Soluble CD25 concentrations in the blood of cynomolgus monkeys injected with BNT152 or BNT153
Cynomolgus monkeys (n=3 per group) were injected IV with 60 or 300 μg/kg BNT152 or 60 or 180 μg/kg BNT153 on Days 1 and 22. Control animals were treated with empty LNPs (lipid dose adapted to 120 μg RNA/kg). Sera were collected for measurement of sCD25 concentrations. Error bars represent standard error of the mean. Vertical dotted lines indicate days of administration. Serum concentrations of sCD25 were strongly increased 2 to 4 days after administration of BNT153 but not BNT152, which induced only moderately elevated sCD25 levels. Serum sCD25 concentrations subsequently declined to levels comparable to those measured in empty LNP-treated animals on Day 21 (pre-dose Cycle 2). Following the second RiboCytokine dosing, sCD25 levels increased with similar kinetics, but with lower peak levels in all groups.

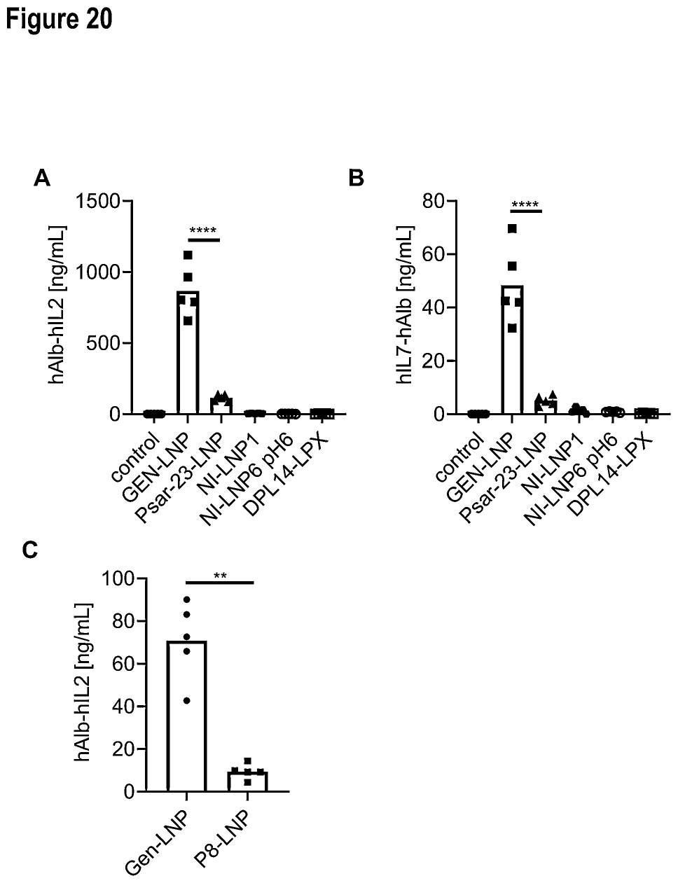

FIG. 20: Gen-LNPs, but not other formulations, confer high exposure to RiboCytokine-encoded proteins
(A, B) Naïve BALB/c mice (n=5 per group) were treated IV with 20 μg gp70 RNA-LPX on Days 0, 7, 14, 21, and 28 in combination with 3 μg hAlb-hIL2 plus 3 μg hlL7-hAlb on Days 7, 14, 21, and 28. hAlb-hIL2- and hIL7-hAlb-encoding RNAs were formulated with either Gen-LNPs, Psar-23 LNP, NI-LNP1, NI LNP6pH6, or DLP14-LPX. Mice treated with NaCl served as negative control. Levels of hAlb-hIL2 (A) and hIL7-hAlb (B) in mouse serum were determined on Day 7, 6 h after administration of RiboCytokines and RNA-LPX. (C) Naïve BALB/c mice (n=5 per group) were treated IV with 1 μg hAlb-hIL2-encoding RNA on Days 0 and 7. The RNA was formulated with either Gen-LNPs or P8-LNPs. hAlb-hIL2 levels in mouse serum were analyzed on Day 7, 5 h after administration of RiboCytokines. The V-PLEX Human IL-2 Kit and the MSD® Multi-Spot Assay System were used for the analysis (A-C). Statistical significance was determined using one-way ANOVA with Tukey's multiple comparisons test. All analyses were two-tailed and carried out using GraphPad Prism 8. ** p≤0.01, **** P≤0.0001. Data are shown as mean. Gen-LNPs enabled the highest serum levels of RiboCytokine-encoded proteins.

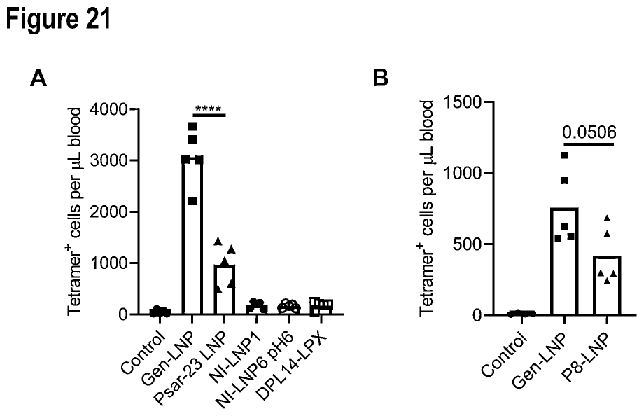

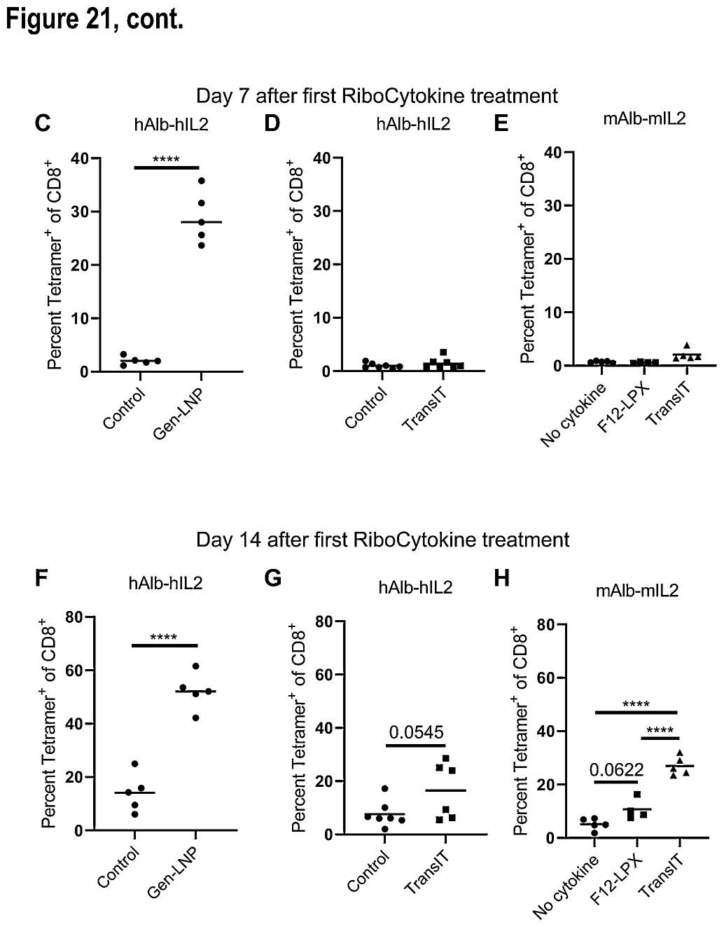

FIG. 21: Gen-LNPs are suitable for obtaining strong RiboCytokine activity and ensure expansion of tumor-specific CD8⁺ T cells
Naïve BALB/c mice (n=5 per group) were either treated as described in FIGS. 20A and B, or were treated IV with 20 μg gp70 RNA-LPX on days 0, 7, 14, and 21, as well as with 3 μg hAlb-hIL2 RNA on days 3, 10, 17, and 24, where hAlb-hIL2 RNA was formulated with either Gen-LNPs or P8-LNPs and mice treated with 10 μg Gen-LNP-formulated RNA encoding hAlb served as negative control (B). (A, B) Numbers of gp70-tetramer+CD8⁺ T cells in the blood on Day 14 were determined by flow cytometry. (C, F) Naïve BALB/c mice (n=5 per group) were treated IV with 20 μg gp70 RNA-LPX on days 0, 7, 14, and 21, as well as with 3 μg hAlb-hIL2-encoding RNA on days 3, 10, 17, and 24. hAlb-hIL2 RNA was formulated with Gen-LNPs. Mice treated with 10 μg Gen-LNP-formulated RNA encoding hAlb served as negative control. (D, G) Naïve BALB/c mice (n=7 per group) were treated IV with 20 μg gp70 RNA-LPX on days 0, 7, and 14, as well as with 1.5 μg hAlb-hIL2 on Day 0, followed by 2.5 μg hAlb-hIL2 on days 7 and 14. hAlb-hIL2 RNA was formulated with TransIT. Mice treated with 1.5 μg hAlb RNA formulated with TransIT served as negative control. (E, H) Naïve BALB/c mice (n=5 per group) were treated IV with 20 μg gp70 RNA-LPX plus 100 μg anti-PD-L1 antibody on days 0 and 7, as well as with 1 μg RNA encoding murine albumin fused to murine IL-2 (mAlb-mIL2) administered on days 2 and 9. mAlb-mIL2 was formulated with either F12-LPX or TransIT. Mice that only received the gp70 RNA-LPX vaccine and anti-PD-L1 antibody served as negative control. (C—H) Frequencies of gp70-tetramer+CD8⁺ T cells in the blood on days 7 and 14 were determined by flow cytometry. Statistical significance was determined using one-way ANOVA with Tukey's multiple comparisons test. All analyses were two-tailed and carried out using GraphPad Prism 8. **** p≤0.0001. Data are shown as Mean. Treatment with Gen-LNP formulations led to the highest frequencies of gp70-specific cells among total CD8⁺ T cells, compared to any other formulation tested.

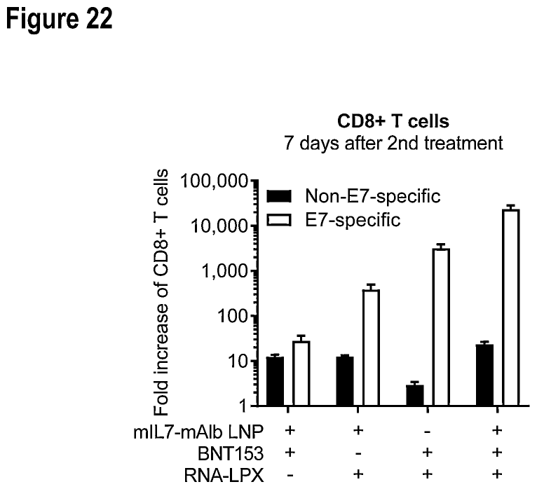

FIG. 22: BNT152 rather than BNT153 expands CD8⁺ T cells with specificities other than the vaccine-encoded antigen, which is boosted by the combination of the two.
TC-1 tumor-bearing C57BL/6 mice (n=10 per group) were treated IV either with 3 μg LNP-formulated RNA encoding hAlb, 3 μg BNT152 mouse surrogate mIL7-mAlb LNP, 3 μg BNT153, or the combination of 3 μg mIL7-mAlb LNP plus 3 μg BNT153, together with 20 μg of an RNA-LPX vaccine encoding the viral tumor antigen E7 or irrelevant RNA-LPX control IV as illustrated in FIG. 14. Fold increase of cell numbers of E7-specific and non-E7-specific CD8⁺ T cells over the median cell number of corresponding subsets in the irrelevant RNA-LPX vaccine only group. Data are shown as mean±SEM.
The combination of mIL7-mAlb LNP and BNT153 with an RNA-LPX vaccine not only induces vaccine-antigen-specific CD8⁺ T cells but also leads to the induction of CD8⁺ T cells specific for antigens other than the vaccine antigen, and thus broadens the anti-tumor CD8⁺ T cell repertoire.

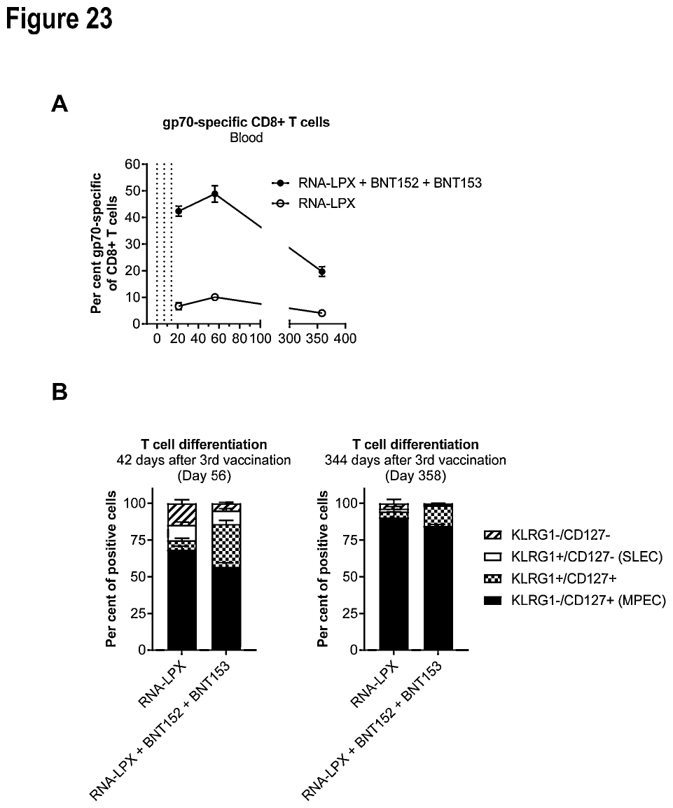

FIG. 23: BNT152 plus BNT153 strongly expands and maintains the antigen-specific T cell memory pool.
BALB/c mice (n=5 per group) were treated IV weekly for 3 weeks (Day 0, 7, and 14) with either the combination of 3 μg BNT152 mouse surrogate mIL7-mAlb LNP plus 3 μg BNT153 together with 20 μg RNA-LPX vaccine encoding the antigen gp70, or with 20 μg RNA-LPX vaccine alone. (A) Fraction of gp70-specific CD8⁺ T cells in the blood at the indicated time points. Vertical dotted lines indicate days of treatment. (B) T cell differentiation phenotype of gp70-specific CD8⁺ T cells in the blood at Day 56 and 358.
The combination of mIL7-mAlb and BNT153 strongly supports proper memory conversion and enhances longevity of the antigen-specific CD8⁺ T cell response.

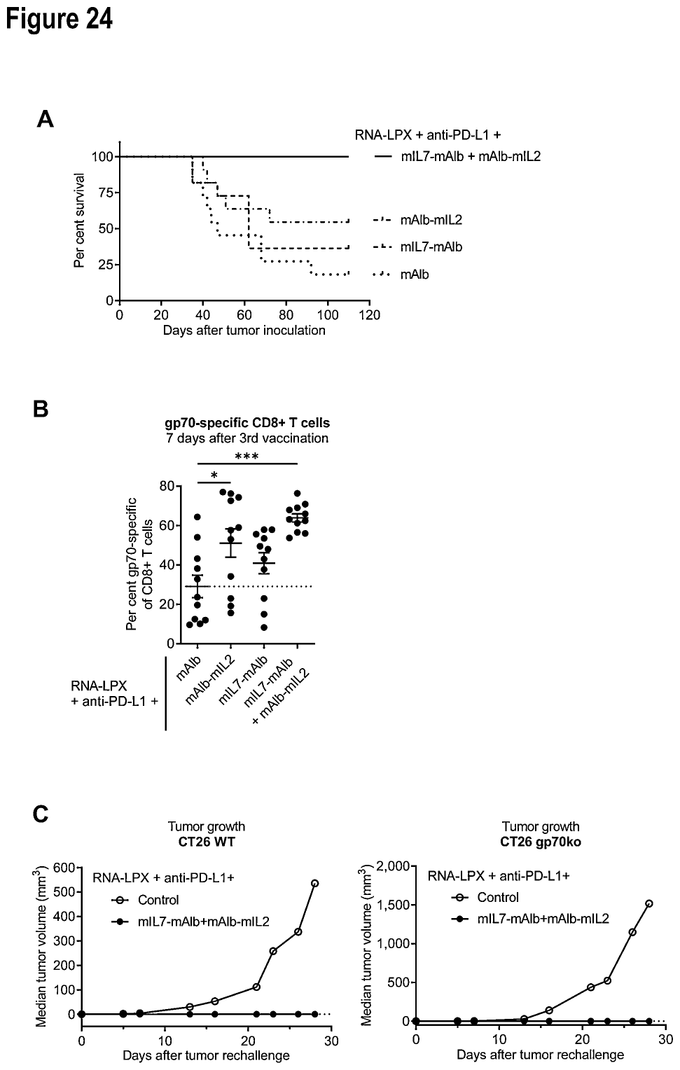

FIG. 24: Treatment with BNT152 plus BNT153 in combination with an RNA-LPX vaccine enables anti-tumor immunity against tumor cells not expressing the vaccine antigen upon tumor rechallenge
BALB/c mice (n=11 per group) were inoculated s.c. with 5×10⁵ syngeneic CT26 wildtype (WT) tumor cells on Day 0 and stratified according to tumor size on Day 13. Mice were treated weekly for six weeks with 20 μg RNA-LPX vaccine encoding the tumor-specific antigen gp70 IV and anti-PD-L1 antibody IP (200 μg loading dose, 100 μg all subsequent doses) (Day 13, 19, 27, 34, 41 and 48), in combination with 1 μg BNT152 mouse surrogate mIL7-mAlb LNP, 1 μg BNT153 mouse surrogate mAlb-mIL2 or the combination of both IV (Day 15, 22, 29, 36, 43 and 50). RNA-LPX vaccine plus anti-PD-L1 antibody in combination with LNP-formulated RNA encoding mAlb served as control. (A) Survival. (B) Fraction of gp70-specific CD8⁺ T cells of total CD8⁺ T cells in the blood seven days after the third vaccination (Day 34). Statistical significance was determined by One-way ANOVA and Holm-Sidak's multiple comparisons test. * p≤0.05, *** p≤0.001, **** p≤0.0001. (C) Surviving mice in the quadruple combination group were rechallenged s.c. with 5×10⁵ CT26 tumor cells either expressing the tumor antigen gp70 (CT26 WT; n=4) or not (CT26 gp70ko; n=5) on Day 133. Untreated BALB/c mice inoculated with either tumor cell line served as controls (n=5 per group). Median tumor volumes until 28 days after rechallenge (Day 161) are depicted.
Mice that had been treated with mIL7-mAlb and mAlb-mIL2 together with RNA-LPX vaccine and anti-PD-L1 antibody challenged with tumor cells that did not express the vaccine antigen gp70 were equally able to fully prevent tumor growth as identically treated mice challenged with tumor cells expressing the vaccine antigen.

### DESCRIPTION OF THE SEQUENCES

The following table provides a listing of certain sequences referenced herein.

DESCRIPTION OF THE SEQUENCES

| | | |
|---|---|---|
| SEQ | | |
| ID | | |
| NO: | Description | SEQUENCE |

hIL7 (mature)

| | | |
|---|---|---|
| 1 | amino acid | DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQV |
| | sequence | KGRKPAALGEAQPTKSLEENKSLKEQKKLNDLCFLKRLLQEIKTCWNKILMGTKEH |

hIL2 (mature)

| | | |
|---|---|---|
| 2 | amino acid | APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSE |
| | sequence | TTFMCEYADETATIVEFLNRWITFCQSIISTLT |

hAlb (mature)

| | | |
|---|---|---|
| 3 | amino acid | DAHKSEVAHRFKDLGEENFKALVLIAFAQYLQQCPFEDHVKLVNEVTEFAKTCVADESAENCDKSLHTLFGDKLCTVATLRETYGEMADCCAKQEP |
| | sequence | ERNECFLQHKDDNPNLPRLVRPEVDVMCTAFHDNEETFLKKYLYEIARRHPYFYAPELLFFAKRYKAAFTECCQAADKAACLLPKLDELRDEGKASS |
| | | AKQRLKCASLQKFGERAFKAWAVARLSQRFPKAEFAEVSKLVTDLTKVHTECCHGDLLECADDRADLAKYICENQDSISSKLKECCEKPLLEKSHCIA |
| | | EVENDEMPADLPSLAADFVESKDVCKNYAEAKDVFLGMFLYEYARRHPDYSVVLLLRLAKTYETTLEKCCAAADPHECYAKVFDEFKPLVEEPQNLI |
| | | KQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNR |
| | | RPCFSALEVDETYVPKEFNAETFTFHADICTLSEKERQIKKQTALVELVKHKPKATKEQLKAVMDDFAAFVEKCCKADDKETCFAEEGKKLVAASQA |
| | | ALGL |

hIL7-hAlb

| | | |
|---|---|---|
| 4 | amino acid | MFHVSFRYIFGLPPLILVLLPVASSDCDIEGKDGKQYESVLMVSIDQLLDSMKEIGSNCLNNEFNFFKRHICDANKEGMFLFRAARKLRQFLKMNST |
| | sequence | GDFDLHLLKVSEGTTILLNCTGQVKGRKPAALGEAQPTKSLEENKSLKEQKKLNDLCFLKRLLQEIKTCWNKILMGTKEHGGSGGGGSGGDAHKSEV |
| | | AHRFKDLGEENFKALVLIAFAQYLQQCPFEDHVKLVNEVTEFAKTCVADESAENCDKSLHTLFGDKLCTVATLRETYGEMADCCAKQEPERNECFLQ |
| | | HKDDNPNLPRLVRPEVDVMCTAFHDNEETFLKKYLYEIARRHPYFYAPELLFFAKRYKAAFTECCQAADKAACLLPKLDELRDEGKASSAKQRLKCA |
| | | SLQKFGERAFKAWAVARLSQRFPKAEFAEVSKLVTDLTKVHTECCHGDLLECADDRADLAKYICENQDSISSKLKECCEKPLLEKSHCIAEVENDEMP |
| | | ADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQ |
| | | LGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVD |
| | | ETYVPKEFNAETFTFHADICTLSEKERQIKKQTALVELVKHKPKATKEQLKAVMDDFAAFVEKCCKADDKETCFAEEGKKLVAASQAALGL |
| 5 | RNA sequence | agacgaacuaguauucuucugguccccacagacucagagagaacccgccaccauguuccauguuucuuuuagguauaucuuuggacuuccuccccugauccuuguucug |
| | | uugccaguagcaucaucugauugugauauugaagguaaagauggcaaacaauaugagaguguucuaauggucagcaucgaucaauuauuggacagcaugaaagaaau |
| | | ugguagcaauugccugaauaaugaauuuaacuuuuuuaaaagacauaucugugaugcuaauaaggaagguauguuuuuauuccgugcugcucgcaaguugaggcaa |
| | | uuucuuaaaaugaauagcacuggugauuuugaucuccacuuauuaaaaguuucagaaggcacaacaauacuguugaacugcacuggccagguuaaaggaagaaaacca |
| | | gcugcccugggugaagcccaaccaacaaagaguuuggaagaaaauaaaucuuuaaaggaacagaaaaaacugaaugacuuguguuuccuaaagagacuauuacaagag |
| | | auaaaaacuuguuggaauaaaauuuugaugggcacuaaagaacacggcggcucuggaggaggcggcuccggaggcgaugcacacaagagugagguugcucaucgcuuua |
| | | aagauuugggagaagaaaauuucaaagccuugguguugauugccuuugcucaguaucuucagcaguguccauuugaagaucauguaaaauuagugaaugaaguaacu |
| | | gaauuugcaaaaacauguguugcugaugagucagcugaaaauugugacaaaucacuucauacccuuuuuggagacaaauuaugcacaguugcaacacuucgugaaacc |
| | | uauggugaaauggcugacugcugugcaaaacaagaaccugagagaaaugaaugcuucuugcaacacaaagaugacaacccaaaccucccccgauuggugagaccagaggu |
| | | ugaugugaugugcacugcuuuucaugacaaugaagaaacauuuuugaaaaaauacuuauaugaaauugccagaagacauccuuacuuuuaugccccggaacuccuuu |
| | | ucuuugcuaaaagguauaaagcugcuuuuacagaauguugccaagcugcugauaaagcugccugccuguugccaaagcucgaugaacuucgggaugaagggaaggcuuc |
| | | gucugccaaacagagacucaagugugccagucuccaaaaauuuggagaaagagcuuucaaagcaugggcaguagcucgccugagccagagauuucccaaagcugaguuug |
| | | cagaaguuuccaaguuagugacagaucuuaccaaaguccacacggaaugcugccauggagaucugcuugaaugugcugaugacagggcggaccuugccaaguauaucug |
| | | ugaaaaucaagauucgaucuccaguaaacugaaggaaugcugugaaaaaccacuguuggaaaaaucccacugcauugccgaaguggaaaaugaugagaugccugcugac |
| | | uugccuucauuagcugcugauuuuguugaaaguaaggauguuugcaaaaacuaugcugaggcaaaggaugucuuccugggcauguuuuuguaugaauaugcaagaag |
| | | gcauccugauuacucugucgugcugcugcugagacuugccaagacauaugaaaccacucuagagaagugcugugccgcugcagauccucaugaaugcuaugccaaagugu |
| | | ucgaugaauuuaaaccucuuguggaggagccucagaauuuaaucaaacaaaauugugagcuuuuugagcagcuuggagaguacaaauuccagaaugcgcuauuaguuc |
| | | guuacaccaagaaaguaccccaagugucaacuccaacucuuguagaggucucaagaaaccuaggaaaagugggcagcaaauguuguaaacauccugaagcaaaaagaau |
| | | gcccugugcagaagacuaucuauccgugguccugaaccaguuauguguguugcaugagaaaacgccaguaagugacagagucaccaaaugcugcacagaauccuuggug |
| | | aacaggcgaccaugcuuuucagcucuggaagucgaugaaacauacguucccaaagaguuuaaugcugaaacauucaccuuccaugcagauauaugcacacuuucugaga |
| | | aggagagacaaaucaagaaacaaacugcacuuguugagcuggugaaacacaagcccaaggcaacaaaagagcaacugaaagcuguuauggaugauuucgcagcuuuugu |
| | | agagaagugcugcaaggcugacgauaaggagaccugcuuugccgaggaggguaaaaaacuuguugcugcaagucaagcugccuuaggcuuaugaugacucgagcuggua |
| | | cugcaugcacgcaaugcuagcugccccuuucccguccuggguaccccgagucucccccgaccucgggucccagguaugcucccaccuccaccugccccacucaccaccucugcu |
| | | aguuccagacaccucccaagcacgcagcaaugcagcucaaaacgcuuagccuagccacacccccacgggaaacagcagugauuaaccuuuagcaauaaacgaaaguuuaac |
| | | uaagcuauacuaaccccaggguuggucaauuucgugccagccacaccgagaccugguccagagucgcuagccgcgucgcuaaaaaaaaaaaaaaaaaaaaaaaaaaaaaa |
| | | gcauaugacuaaaaaaaaaaaaaaaaaaaaaaaaaaaaaaaaaaaaaaaaaaaaaaaaaaaaaaaaaaaaaaaaaaaaaa |

hAlb-hIL2

| | | |
|---|---|---|
| 6 | amino acid | MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDK |
| | sequence | LCTVATLRETYGEMADCCAKQEPERNECFLQHKDDNPNLPRLVRPEVDVMCTAFHDNEETFLKKYLYEIARRHPYFYAPELLFFAKRYKAAFTECCQ |
| | | AADKAACLLPKLDELRDEGKASSAKQRLKCASLQKFGERAFKAWAVARLSQRFPKAEFAEVSKLVTDLTKVHTECCHGDLLECADDRADLAKYICE |
| | | NQDSISSKLKECCEKPLLEKSHCIAEVENDEMPADLPSLAADFVESKDVCKNYAEAKDVFLGMFLYEYARRHPDYSVVLLLRLAKTYETTLEKCCAAA |
| | | DPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLC |
| | | VLHEKTPVSDRVTKCCTESLVNRRPCFSALEVDETYVPKEFNAETFTFHADICTLSEKERQIKKQTALVELVKHKPKATKEQLKAVMDDFAAFVEKCC |
| | | KADDKETCFAEEGKKLVAASQAALGLGGSGGGGSGGAPTSSSTKKTQLQLEHLLLDLQMILNGINNYKNPKLTRMLTFKFYMPKKATELKHLQCLE |
| | | EELKPLEEVLNLAQSKNFHLRPRDLISNINVIVLELKGSETTFMCEYADETATIVEFLNRWITFCQSIISTLT |
| 7 | RNA sequence | agacgaacuaguauucuucugguccccacagacucagagagaacccgccaccaugaaguggguaaccuuuauuucccuucuuuuucucuuuagcucggcuuauuccagg |
| | | gguguguuucgucgagaugcacacaagagugagguugcucaucgcuuuaaagauuugggagaagaaaauuucaaagccuugguguugauugccuuugcucaguaucu |
| | | ucagcaguguccauuugaagaucauguaaaauuagugaaugaaguaacugaauuugcaaaaacauguguugcugaugagucagcugaaaauugugacaaaucacuuc |
| | | auacccuuuuuggagacaaauuaugcacaguugcaacacuucgugaaaccuauggugaaauggcugacugcugugcaaaacaagaaccugagagaaaugaaugcuucu |
| | | ugcaacacaaagaugacaacccaaaccucccccgauuggugagaccagagguugaugugaugugcacugcuuuucaugacaaugaagaaacauuuuugaaaaaauacuu |
| | | auaugaaauugccagaagacauccuuacuuuuaugccccggaacuccuuuucuuugcuaaaagguauaaagcugcuuuuacagaauguugccaagcugcugauaaagc |
| | | ugccugccuguugccaaagcucgaugaacuucgggaugaagggaaggcuucgucugccaaacagagacucaagugugccagucuccaaaaauuuggagaaagagcuuuca |
| | | aagcaugggcaguagcucgccugagccagagauuucccaaagcugaguuugcagaaguuuccaaguuagugacagaucuuaccaaaguccacacggaaugcugccaugga |
| | | gaucugcuugaaugugcugaugacagggcggaccuugccaaguauaucugugaaaaucaagauucgaucuccaguaaacugaaggaaugcugugaaaaaccacuguug |
| | | gaaaaaucccacugcauugccgaaguggaaaaugaugagaugccugcugacuugccuucauuagcugcugauuuuguugaaaguaaggauguuugcaaaaacuaugcu |
| | | gaggcaaaggaugucuuccugggcauguuuuuguaugaauaugcaagaaggcauccugauuacucugucgugcugcugcugagacuugccaagacauaugaaaccacuc |
| | | agcuuuuugagcagcuuggagaguacaaauuccagaaugcgcuauuaguucguuacaccaagaaaguaccccaagugucaacuccaacucuuguagaggucucaagaaa |
| | | uagagaagugcugugccgcugcagauccucaugaaugcuaugccaaaguguucgaugaauuuaaaccucuuguggaggagccucagaauuuaaucaaacaaaauugug |
| | | ccuaggaaaagugggcagcaaauguuguaaacauccugaagcaaaaagaaugcccugugcagaagacuaucuauccgugguccugaaccaguuauguguguugcaugag |
| | | aaaacgccaguaagugacagagucaccaaaugcugcacagaauccuuggugaacaggcgaccaugcuuuucagcucuggaagucgaugaaacauacguucccaaagaguu |
| | | uaaugcugaaacauucaccuuccaugcagauauaugcacacuuucugagaaggagagacaaaucaagaaacaaacugcacuuguugagcuggugaaacacaagcccaag |
| | | gcaacaaaagagcaacugaaagcuguuauggaugauuucgcagcuuuuguagagaagugcugcaaggcugacgauaaggagaccugcuuugccgaggaggguaaaaaac |
| | | uuguugcugcaagucaagcugccuuaggcuuaggcggcucuggaggaggcggcuccggaggcgcuccaacaucuucuucaacaaagaaaacacagcuucagcuugaacacc |
| | | uucuucuugaucuucagaugauucugaauggaaucaacaauuacaaaaauccaaaacugacaagaaugcugacauuuaaauuuuacaugccaaagaaagcaacagaac |
| | | ugaaacaccuucagugccuugaagaagaacugaaaccucuggaagaagugcugaaucuggcucagagcaaaaauuuucaccugagaccaagagaucugaucagcaacau |
| | | caaugugauugugcuggaacugaaaggaucugaaacaacauucaugugugaauaugcugaugaaacagcaacaauuguggaauuucugaacagauggauuacauuuu |
| | | gccagucaaucauuucaacacugacaugaugacucgagcugguacugcaugcacgcaaugcuagcugccccuuucccguccuggguaccccgagucucccccgaccucgggu |
| | | cccagguaugcucccaccuccaccugccccacucaccaccucugcuaguuccagacaccucccaagcacgcagcaaugcagcucaaaacgcuuagccuagccacacccccacgg |
| | | gaaacagcagugauuaaccuuuagcaauaaacgaaaguuuaacuaagcuauacuaaccccaggguuggucaauuucgugccagccacaccgagaccugguccagagucgc |
| | | uagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaa |
| | | aaaaaaaaaaaaaaa |

Sec/MITD

| | | |
|---|---|---|
| 8 | Sec (amino acid) | MRVMAPRTLILLLSGALALTETWAGS |
| 9 | MITD (amino | IVGIVAGLAVLAVVVIGAVVATVMCRRKSSGGKGGSYSQAASSDSAQGSDVSLTA |
| | acid) | |

P2P16

| | | |
|---|---|---|
| 10 | P2P16 (amino | KKQYIKANSKFIGITELKKLGGGKRGGGKKMTNSVDDALINSTKIYSYFPSVISKVNQGAQGKKL |
| | acid) | |

GS Linker

| | | |
|---|---|---|
| 11 | GS Linker 1 | GGSGGGGSGG |
| 12 | GS Linker 2 | GSSGGGGSPGGGSS |

5′-UTR (hAg-Kozak)

| | | |
|---|---|---|
| 13 | 5'-UTR | AACUAGUAUUCUUCUGGUCCCCACAGACUCAGAGAGAACCCGCCACC |

3′-UTR (FI element)

| | | |
|---|---|---|
| 14 | 3'-UTR | CUGGUACUGCAUGCACGCAAUGCUAGCUGCCCCUUUCCCGUCCUGGGUACCCCGAGUCUCCCCCGACCUCGGGUCCCAGGUAUGCUCCC |
| | | ACCUCCACCUGCCCCACUCACCACCUCUGCUAGUUCCAGACACCUCCCAAGCACGCAGCAAUGCAGCUCAAAACGCUUAGCCUAGCCACA |
| | | CCCCCACGGGAAACAGCAGUGAUUAACCUUUAGCAAUAAACGAAAGUUUAACUAAGCUAUACUAACCCCAGGGUUGGUCAAUUUCGU |
| | | GCCAGCCACACC |

A30L70

| | | |
|---|---|---|
| 15 | A30L70 | AAAAAAAAAAAAAAAAAAAAAAAAAAAAAAGCAUAUGACUAAAAAAAAAAAAAAAAAAAAAAAAAAAAAAAAAAAAAAAAAAAAAAA |
| | | AAAAAAAAAAAAAAAAAAAAAAA |

Helper epitopes

| | | |
|---|---|---|
| 16 | P2 | QYIKANSKFIGITEL |
| 17 | P16 | MTNSVDDALINSTKIYSYFPSVISKVNQGAQG |

### Detailed Description

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and h. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of” or “consisting essentially of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero.

Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, immunostimulants and antigens for vaccination.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS:needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq) . In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1,-2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to immunostimulants, one particular function is one or more immunostimulatory activities displayed by the amino acid sequence from which the fragment or variant is derived. With respect to antigens or antigenic sequences, one particular function is one or more immunogenic activities (e.g., specificity of the immune reaction) displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., stimulating or inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunostimulatory activity or immunogenicity of the functional variant may be at least 50%, at least 60%, at least 70%, at least 80%, or at least 90% of the parent molecule or sequence. However, in other embodiments, function of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the amino acid sequences suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant nucleic acid in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

“Physiological pH” as used herein refers to a pH of about 7.5.

The term “genetic modification” or simply “modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding immunostimulant or antigen is transiently transfected into cells. RNA can be transfected into cells to transiently express its coded protein.

### Immunostimulants

The present invention comprises the use of RNA encoding an amino acid sequence comprising hIL7, a functional variant thereof, or a functional fragment of the hIL7 or the functional variant thereof. Alternatively or additionally, the present invention comprises the use of RNA encoding an amino acid sequence comprising hIL2, a functional variant thereof, or a functional fragment of the hIL2 or the functional variant thereof.

The methods and agents described herein are particularly effective if the immunostimulant portion is attached to a pharmacokinetic modifying group (hereafter referred to as “extended-pharmacokinetic (PK)” immunostimulant). In one embodiment, said RNA is targeted to the liver for systemic availability. Liver cells can be efficiently transfected and are able to produce large amounts of protein.

An “immunostimulant” is any substance that stimulates the immune system by inducing activation or increasing activity of any of the immune system's components, in particular immune effector cells.

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons (IFNs), interleukins, lymphokines, and tumor necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

Interleukins are a group of cytokines (secreted proteins and signal molecules) that can be divided into four major groups based on distinguishing structural features. However, their amino acid sequence similarity is rather weak (typically 15-25% identity). The human genome encodes more than 50 interleukins and related proteins.

IL7 is a hematopoietic growth factor secreted by stromal cells in the bone marrow and thymus. It is also produced by keratinocytes, dendritic cells, hepatocytes, neurons, and epithelial cells, but is not produced by normal lymphocytes. IL7 is a cytokine important for B and T cell development. IL7 cytokine and the hepatocyte growth factor form a heterodimer that functions as a pre-pro-B cell growth-stimulating factor. Knockout studies in mice suggested that IL7 plays an essential role in lymphoid cell survival.

IL7 binds to the IL7 receptor, a heterodimer consisting of IL7 receptor a and common y chain receptor. Binding results in a cascade of signals important for T-cell development within the thymus and survival within the periphery. Knockout mice which genetically lack IL7 receptor exhibit thymic atrophy, arrest of T-cell development at the double positive stage, and severe lymphopenia. Administration of IL7 to mice results in an increase in recent thymic emigrants, increases in B and T cells, and increased recovery of T cells after cyclophosphamide administration or after bone marrow transplantation.

According to the disclosure, human IL7 (hIL7) (optionally as a portion of extended-PK hIL7) may be naturally occurring hIL7 or a fragment or variant thereof. In one embodiment, hIL7 comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In one embodiment, hIL7 or a hIL7 fragment or variant binds to the IL7 receptor.

According to the disclosure, in certain embodiments, hIL7 is attached to a pharmacokinetic modifying group. The resulting molecule, hereafter referred to as “extended-pharmacokinetic (PK) hIL7,” has a prolonged circulation half-life relative to free hIL7. The prolonged circulation half-life of extended-PK hIL7 permits in vivo serum hIL7 concentrations to be maintained within a therapeutic range, potentially leading to the enhanced activation of many types of immune cells, including T cells. Because of its favorable pharmacokinetic profile, extended-PK hIL7 can be dosed less frequently and for longer periods of time when compared with unmodified hIL7. In certain embodiments, the pharmacokinetic modifying group of the extended-PK hIL7 is human albumin (hAlb).

In one embodiment, hAlb comprises the amino acid sequence of SEQ ID NO: 3, an amino acid sequence having at least 99%, 98%, 97%, 96%, 95%, 90%, 85%, or 80% identity to the amino acid sequence of SEQ ID NO: 3, or a functional fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3.

Interleukin-2 (IL2) is a cytokine that induces proliferation of antigen-activated T cells and stimulates natural killer (NK) cells. The biological activity of IL2 is mediated through a multi-subunit IL2 receptor complex (IL2R) of three polypeptide subunits that span the cell membrane: p55 (IL2Ra, the alpha subunit, also known as CD25 in humans), p75 (IL2RB, the beta subunit, also known as CD122 in humans) and p64 (IL2Ry, the gamma subunit, also known as CD 132 in humans). T cell response to IL2 depends on a variety of factors, including: (1) the concentration of IL2; (2) the number of IL2R molecules on the cell surface; and (3) the number of IL2R occupied by IL2 (i.e., the affinity of the binding interaction between IL2 and IL2R (Smith, “Cell Growth Signal Transduction is Quantal” In Receptor Activation by Antigens, Cytokines, Hormones, and Growth Factors 766:263-271, 1995)). The IL2:IL2R complex is internalized upon ligand binding and the different components undergo differential sorting. When administered as an IV bolus, IL2 has a rapid systemic clearance (an initial clearance phase with a half-life of 12.9 minutes followed by a slower clearance phase with a half-life of 85 minutes) (Konrad et al., Cancer Res. 50:2009-2017, 1990).

Outcomes of systemic IL2 administration in cancer patients are far from ideal. While 15 to 20 percent of patients respond objectively to high-dose IL2, the great majority do not, and many suffer severe, life-threatening side effects, including nausea, confusion, hypotension, and septic shock. Attempts to reduce serum concentration by reducing dose and adjusting dosing regimen have been attempted, and while less toxic, such treatments were also less efficacious.

According to the disclosure, human IL2 (hIL2) (optionally as a portion of extended-PK hIL2) may be naturally occurring hIL2 or a fragment or variant thereof. In one embodiment, hIL2 comprises the amino acid sequence of SEQ ID NO: 2, an amino acid sequence having at least 99%, 98%, 97%, 96%, 95%, 90%, 85%, or 80% identity to the amino acid sequence of SEQ ID NO: 2, or a functional fragment of the amino acid sequence of SEQ ID NO: 2, or the amino acid sequence having at least 99%, 98%, 97%, 96%, 95%, 90%, 85%, or 80% identity to the amino acid sequence of SEQ ID NO: 2. In one embodiment, hIL2 or a hIL2 fragment or variant binds to the IL2 receptor.

According to the disclosure, in certain embodiments, hIL2 is attached to a pharmacokinetic modifying group. The resulting molecule, hereafter referred to as “extended-pharmacokinetic (PK) hIL2,” has a prolonged circulation half-life relative to free hIL2. The prolonged circulation half-life of extended-PK hIL2 permits in vivo serum hIL2 concentrations to be maintained within a therapeutic range, potentially leading to the enhanced activation of many types of immune cells, including T cells. Because of its favorable pharmacokinetic profile, extended-PK hIL2 can be dosed less frequently and for longer periods of time when compared with unmodified hIL2. In certain embodiments, the pharmacokinetic modifying group of the extended-PK hIL2 is human albumin (hAlb).

In one embodiment, hAlb comprises the amino acid sequence of SEQ ID NO: 3, an amino acid sequence having at least 99%, 98%, 97%, 96%, 95%, 90%, 85%, or 80% identity to the amino acid sequence of SEQ ID NO: 3, or a functional fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3.

The immunostimulant RNA described herein encodes a polypeptide comprising an immunostimulant portion. The immunostimulant portion may be a hIL7-derived immunostimulant portion or hIL7 immunostimulant portion and/or a hIL2-derived immunostimulant portion or hIL2 immunostimulant portion. The hIL7 immunostimulant portion may be hIL7, a functional variant thereof, or a functional fragment of the hIL7 or the functional variant thereof. The hIL2 immunostimulant portion may be hIL2, a functional variant thereof, or a functional fragment of the hIL2 or the functional variant thereof.

Thus, the polypeptide comprising an immunostimulant portion may be a hIL7 immunostimulant polypeptide (also designated herein “amino acid sequence comprising human IL7 (hIL7), a functional variant thereof, or a functional fragment of the hIL7 or the functional variant thereof”) or a hIL2 immunostimulant polypeptide (also designated herein “amino acid sequence comprising human IL2 (hIL2), a functional variant thereof, or a functional fragment of the hIL2 or the functional variant thereof”).

In one embodiment, a hIL7 immunostimulant polypeptide comprises the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In one embodiment, a hIL7 immunostimulant polypeptide comprises the amino acid sequence of SEQ ID NO: 1.

In one embodiment, RNA encoding a hIL7 immunostimulant polypeptide (i) comprises the nucleotide sequence of nucleotides 128 to 583 of SEQ ID NO: 5, a nucleotide sequence having at least 99%, 98%, 97%, 96%, 95%, 90%, 85%, or 80% identity to the nucleotide sequence of nucleotides 128 to 583 of SEQ ID NO: 5, or a fragment of the nucleotide sequence of nucleotides 128 to 583 of SEQ ID NO: 5, or the nucleotide sequence having at least 99%, 98%, 97%, 96%, 95%, 90%, 85%, or 80% identity to the nucleotide sequence of nucleotides 128 to 583 of SEQ ID NO: 5; and/or (ii) encodes an amino acid sequence comprising the amino acid sequence of SEQ ID NO: 1, an amino acid sequence having at least 99%, 98%, 97%, 96%, 95%, 90%, 85%, or 80% identity to the amino acid sequence of SEQ ID NO: 1, or a functional fragment of the amino acid sequence of SEQ ID NO: 1, or the amino acid sequence having at least 99%, 98%, 97%, 96%, 95%, 90%, 85%, or 80% identity to the amino acid sequence of SEQ ID NO: 1. In one embodiment, RNA encoding a hIL7 immunostimulant polypeptide (i) comprises the nucleotide sequence of nucleotides 128 to 583 of SEQ ID NO: 5; and/or (ii) encodes an amino acid sequence comprising the amino acid sequence of SEQ ID NO: 1.

In one embodiment, a hIL7 immunostimulant polypeptide comprises the amino acid sequence of amino acids 1 to 177 of SEQ ID NO: 4, an amino acid sequence having at least 99%, 98%, 97%, 96%, 95%, 90%, 85%, or 80% identity to the amino acid sequence of amino acids 1 to 177 of SEQ ID NO: 4, or a functional fragment of the amino acid sequence of amino acids 1 to 177 of SEQ ID NO: 4, or the amino acid sequence having at least 99%, 98%, 97%, 96%, 95%, 90%, 85%, or 80% identity to the amino acid sequence of amino acids 1 to 177 of SEQ ID NO: 4. In one embodiment, a hIL7 immunostimulant polypeptide comprises the amino acid sequence of amino acids 1 to 177 of SEQ ID NO: 4.

In one embodiment, RNA encoding a hIL7 immunostimulant polypeptide (i) comprises the nucleotide sequence of nucleotides 53 to 583 of SEQ ID NO: 5, a nucleotide sequence having at least 99%, 98%, 97%, 96%, 95%, 90%, 85%, or 80% identity to the nucleotide sequence of nucleotides 53 to 583 of SEQ ID NO: 5, or a fragment of the nucleotide sequence of nucleotides 53 to 583 of SEQ ID NO: 5, or the nucleotide sequence having at least 99%, 98%, 97%, 96%, 95%, 90%, 85%, or 80% identity to the nucleotide sequence of nucleotides 53 to 583 of SEQ ID NO: 5; and/or (ii) encodes an amino acid sequence comprising the amino acid sequence of amino acids 1 to 177 of SEQ ID NO: 4, an amino acid sequence having at least 99%, 98%, 97%, 96%, 95%, 90%, 85%, or 80% identity to the amino acid sequence of amino acids 1 to 177 of SEQ ID NO: 4, or a functional fragment of the amino acid sequence of amino acids 1 to 177 of SEQ ID NO: 4, or the amino acid sequence having at least 99%, 98%, 97%, 96%, 95%, 90%, 85%, or 80% identity to the amino acid sequence of amino acids 1 to 177 of SEQ ID NO: 4. In one embodiment, RNA encoding a hIL7 immunostimulant polypeptide (i) comprises the nucleotide sequence of nucleotides 53 to 583 of SEQ ID NO: 5; and/or (ii) encodes an amino acid sequence comprising the amino acid sequence of amino acids 1 to 177 of SEQ ID NO: 4.

In one embodiment, a hIL2 immunostimulant polypeptide comprises the amino acid sequence of SEQ ID NO: 2, an amino acid sequence having at least 99%, 98%, 97%, 96%, 95%, 90%, 85%, or 80% identity to the amino acid sequence of SEQ ID NO: 2, or a functional fragment of the amino acid sequence of SEQ ID NO: 2, or the amino acid sequence having at least 99%, 98%, 97%, 96%, 95%, 90%, 85%, or 80% identity to the amino acid sequence of SEQ ID NO: 2. In one embodiment, a hIL2 immunostimulant polypeptide comprises the amino acid sequence of SEQ ID NO: 2.

In one embodiment, RNA encoding a hIL2 immunostimulant polypeptide (i) comprises the nucleotide sequence of nucleotides 1910 to 2308 of SEQ ID NO: 7, a nucleotide sequence having at least 99%, 98%, 97%, 96%, 95%, 90%, 85%, or 80% identity to the nucleotide sequence of nucleotides 1910 to 2308 of SEQ ID NO: 7, or a fragment of the nucleotide sequence of nucleotides 1910 to 2308 of SEQ ID NO: 7, or the nucleotide sequence having at least 99%, 98%, 97%, 96%, 95%, 90%, 85%, or 80% identity to the nucleotide sequence of nucleotides 1910 to 2308 of SEQ ID NO: 7; and/or (ii) encodes an amino acid sequence comprising the amino acid sequence of SEQ ID NO: 2, an amino acid sequence having at least 99%, 98%, 97%, 96%, 95%, 90%, 85%, or 80% identity to the amino acid sequence of SEQ ID NO: 2, or a functional fragment of the amino acid sequence of SEQ ID NO: 2, or the amino acid sequence having at least 99%, 98%, 97%, 96%, 95%, 90%, 85%, or 80% identity to the amino acid sequence of SEQ ID NO: 2. In one embodiment, RNA encoding a hIL2 immunostimulant polypeptide (i) comprises the nucleotide sequence of nucleotides 1910 to 2308 of SEQ ID NO: 7; and/or (ii) encodes an amino acid sequence comprising the amino acid sequence of SEQ ID NO: 2.

In one embodiment, hAlb is fused, either directly or through a linker, to an immunostimulant portion.

In one embodiment, hAlb comprises the amino acid sequence of SEQ ID NO: 3, an amino acid sequence having at least 99%, 98%, 97%, 96%, 95%, 90%, 85%, or 80% identity to the amino acid sequence of SEQ ID NO: 3, or a functional fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3. In one embodiment, hAlb comprises the amino acid sequence of SEQ ID NO: 3.

In one embodiment, RNA encoding hAlb (i) comprises the nucleotide sequence of nucleotides 614 to 2368 of SEQ ID NO: 5, a nucleotide sequence having at least 99%, 98%, 97%, 96%, 95%, 90%, 85%, or 80% identity to the nucleotide sequence of nucleotides 614 to 2368 of SEQ ID NO: 5, or a fragment of the nucleotide sequence of nucleotides 614 to 2368 of SEQ ID NO: 5, or the nucleotide sequence having at least 99%, 98%, 97%, 96%, 95%, 90%, 85%, or 80% identity to the nucleotide sequence of nucleotides 614 to 2368 of SEQ ID NO: 5; and/or (ii) encodes an amino acid sequence comprising the amino acid sequence of SEQ ID NO: 3, an amino acid sequence having at least 99%, 98%, 97%, 96%, 95%, 90%, 85%, or 80% identity to the amino acid sequence of SEQ ID NO: 3, or a functional fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3. In one embodiment, RNA encoding hAlb (i) comprises the nucleotide sequence of nucleotides 614 to 2368 of SEQ ID NO: 5; and/or (ii) encodes an amino acid sequence comprising the amino acid sequence of SEQ ID NO: 3.

hAlb is preferably used in order to promote prolonged circulation half-life of the immunostimulant portion. Accordingly, in particularly preferred embodiments, the immunostimulant RNA described herein comprises at least one coding region encoding an immunostimulant portion and a coding region encoding hAlb, said hAlb preferably being fused to the immunostimulant portion, e.g., to the N-terminus and/or the C-terminus of the immunostimulant portion. In one embodiment, hAlb and the immunostimulant portion are separated by a linker such as a GS linker, e.g. a GS linker having the amino acid sequence of SEQ ID NO: 11.

In one embodiment, a hIL7 immunostimulant polypeptide comprises the amino acid sequence of amino acids 26 to 772 of SEQ ID NO: 4, an amino acid sequence having at least 99%, 98%, 97%, 96%, 95%, 90%, 85%, or 80% identity to the amino acid sequence of amino acids 26 to 772 of SEQ ID NO: 4, or a functional fragment of the amino acid sequence of amino acids 26 to 772 of SEQ ID NO: 4, or the amino acid sequence having at least 99%, 98%, 97%, 96%, 95%, 90%, 85%, or 80% identity to the amino acid sequence of amino acids 26 to 772 of SEQ ID NO: 4. In one embodiment, a hIL7 immunostimulant polypeptide comprises the amino acid sequence of amino acids 26 to 772 of SEQ ID NO: 4.

In one embodiment, RNA encoding a hIL7 immunostimulant polypeptide (i) comprises the nucleotide sequence of nucleotides 128 to 2368 of SEQ ID NO: 5, a nucleotide sequence having at least 99%, 98%, 97%, 96%, 95%, 90%, 85%, or 80% identity to the nucleotide sequence of nucleotides 128 to 2368 of SEQ ID NO: 5, or a fragment of the nucleotide sequence of nucleotides 128 to 2368 of SEQ ID NO: 5, or the nucleotide sequence having at least 99%, 98%, 97%, 96%, 95%, 90%, 85%, or 80% identity to the nucleotide sequence of nucleotides 128 to 2368 of SEQ ID NO: 5; and/or (ii) encodes an amino acid sequence comprising the amino acid sequence of amino acids 26 to 772 of SEQ ID NO: 4, an amino acid sequence having at least 99%, 98%, 97%, 96%, 95%, 90%, 85%, or 80% identity to the amino acid sequence of amino acids 26 to 772 of SEQ ID NO: 4, or a functional fragment of the amino acid sequence of amino acids 26 to 772 of SEQ ID NO: 4, or the amino acid sequence having at least 99%, 98%, 97%, 96%, 95%, 90%, 85%, or 80% identity to the amino acid sequence of amino acids 26 to 772 of SEQ ID NO: 4. In one embodiment, RNA encoding a hIL7 immunostimulant polypeptide (i) comprises the nucleotide sequence of nucleotides 128 to 2368 of SEQ ID NO: 5; and/or (ii) encodes an amino acid sequence comprising the amino acid sequence of amino acids 26 to 772 of SEQ ID NO: 4.

In one embodiment, a hIL2 immunostimulant polypeptide comprises the amino acid sequence of amino acids 25 to 752 of SEQ ID NO: 6, an amino acid sequence having at least 99%, 98%, 97%, 96%, 95%, 90%, 85%, or 80% identity to the amino acid sequence of amino acids 25 to 752 of SEQ ID NO: 6, or a functional fragment of the amino acid sequence of amino acids 25 to 752 of SEQ ID NO: 6, or the amino acid sequence having at least 99%, 98%, 97%, 96%, 95%, 90%, 85%, or 80% identity to the amino acid sequence of amino acids 25 to 752 of SEQ ID NO: 6. In one embodiment, a hIL2 immunostimulant polypeptide comprises the amino acid sequence of amino acids 25 to 752 of SEQ ID NO: 6.

In one embodiment, RNA encoding a hIL2 immunostimulant polypeptide (i) comprises the nucleotide sequence of nucleotides 125 to 2308 of SEQ ID NO: 7, a nucleotide sequence having at least 99%, 98%, 97%, 96%, 95%, 90%, 85%, or 80% identity to the nucleotide sequence of nucleotides 125 to 2308 of SEQ ID NO: 7, or a fragment of the nucleotide sequence of nucleotides 125 to 2308 of SEQ ID NO: 7, or the nucleotide sequence having at least 99%, 98%, 97%, 96%, 95%, 90%, 85%, or 80% identity to the nucleotide sequence of nucleotides 125 to 2308 of SEQ ID NO: 7; and/or (ii) encodes an amino acid sequence comprising the amino acid sequence of amino acids 25 to 752 of SEQ ID NO: 6, an amino acid sequence having at least 99%, 98%, 97%, 96%, 95%, 90%, 85%, or 80% identity to the amino acid sequence of amino acids 25 to 752 of SEQ ID NO: 6, or a functional fragment of the amino acid sequence of amino acids 25 to 752 of SEQ ID NO: 6, or the amino acid sequence having at least 99%, 98%, 97%, 96%, 95%, 90%, 85%, or 80% identity to the amino acid sequence of amino acids 25 to 752 of SEQ ID NO: 6. In one embodiment, RNA encoding a hIL2 immunostimulant polypeptide (i) comprises the nucleotide sequence of nucleotides 125 to 2308 of SEQ ID NO: 7; and/or (ii) encodes an amino acid sequence comprising the amino acid sequence of amino acids 25 to 752 of SEQ ID NO: 6.

According to certain embodiments, a signal peptide is fused, either directly or through a linker, to an immunostimulant portion which is optionally fused to hAlb.

Such signal peptides are sequences, which typically exhibit a length of about 15 to 30 amino acids and are preferably located at the N-terminus of the polypeptide to which it is fused, without being limited thereto. Signal peptides as defined herein preferably allow the transport of the peptide or protein it is fused to into a defined cellular compartment, preferably the cell surface, the endoplasmic reticulum (ER) or the endosomal-lysosomal compartment.

In one embodiment, the signal peptide sequence as defined herein includes, without being limited thereto, the signal peptide sequence of an interleukin. In one embodiment, the signal peptide sequence as defined herein includes, without being limited thereto, the signal peptide sequence of the interleukin from which the immunostimulant portion is derived, in particular if the immunostimulant portion is the N-terminal portion of the immunostimulant polypeptide. Accordingly, the immunostimulant portion may be the non-mature IL, i.e., the IL containing its endogenous signal peptide.

In one embodiment, the signal peptide sequence as defined herein includes, without being limited thereto, the signal peptide sequence of an extended-PK group, e.g., albumin. In one embodiment, the signal peptide sequence as defined herein includes, without being limited thereto, the signal peptide sequence of the extended-PK group, e.g., albumin, from which the extended-PK group, e.g., albumin, is derived, in particular if the extended-PK group, e.g., albumin, is the N-terminal portion of the immunostimulant polypeptide. Accordingly, the extended-PK group, e.g., albumin, may be the non-mature extended-PK group, e.g., albumin, i.e., the extended-PK group, e.g., albumin, containing its endogenous signal peptide.

In one embodiment, a signal sequence comprises the amino acid sequence of amino acids 1 to 25 of SEQ ID NO: 4, an amino acid sequence having at least 99%, 98%, 97%, 96%, 95%, 90%, 85%, or 80% identity to the amino acid sequence of amino acids 1 to 25 of SEQ ID NO: 4, or a functional fragment of the amino acid sequence of amino acids 1 to 25 of SEQ ID NO: 4, or the amino acid sequence having at least 99%, 98%, 97%, 96%, 95%, 90%, 85%, or 80% identity to the amino acid sequence of amino acids 1 to 25 of SEQ ID NO: 4. In one embodiment, a signal sequence comprises the amino acid sequence of amino acids 1 to 25 of SEQ ID NO: 4.

In one embodiment, RNA encoding a signal sequence (i) comprises the nucleotide sequence of nucleotides 53 to 127 of SEQ ID NO: 5, a nucleotide sequence having at least 99%, 98%, 97%, 96%, 95%, 90%, 85%, or 80% identity to the nucleotide sequence of nucleotides 53 to 127 of SEQ ID NO: 5, or a fragment of the nucleotide sequence of nucleotides 53 to 127 of SEQ ID NO: 5, or the nucleotide sequence having at least 99%, 98%, 97%, 96%, 95%, 90%, 85%, or 80% identity to the nucleotide sequence of nucleotides 53 to 127 of SEQ ID NO: 5; and/or (ii) encodes an amino acid sequence comprising the amino acid sequence of amino acids 1 to 25 of SEQ ID NO: 4, an amino acid sequence having at least 99%, 98%, 97%, 96%, 95%, 90%, 85%, or 80% identity to the amino acid sequence of amino acids 1 to 25 of SEQ ID NO: 4, or a functional fragment of the amino acid sequence of amino acids 1 to 25 of SEQ ID NO: 4, or the amino acid sequence having at least 99%, 98%, 97%, 96%, 95%, 90%, 85%, or 80% identity to the amino acid sequence of amino acids 1 to 25 of SEQ ID NO: 4. In one embodiment, RNA encoding a signal sequence (i) comprises the nucleotide sequence of nucleotides 53 to 127 of SEQ ID NO: 5; and/or (ii) encodes an amino acid sequence comprising the amino acid sequence of amino acids 1 to 25 of SEQ ID NO: 4.

In one embodiment, a signal sequence comprises the amino acid sequence of amino acids 1 to 18 of SEQ ID NO: 6, an amino acid sequence having at least 99%, 98%, 97%, 96%, 95%, 90%, 85%, or 80% identity to the amino acid sequence of amino acids 1 to 18 of SEQ ID NO: 6, or a functional fragment of the amino acid sequence of amino acids 1 to 18 of SEQ ID NO: 6, or the amino acid sequence having at least 99%, 98%, 97%, 96%, 95%, 90%, 85%, or 80% identity to the amino acid sequence of amino acids 1 to 18 of SEQ ID NO: 6. In one embodiment, a signal sequence comprises the amino acid sequence of amino acids 1 to 18 of SEQ ID NO: 6.

In one embodiment, RNA encoding a signal sequence (i) comprises the nucleotide sequence of nucleotides 53 to 106 of SEQ ID NO: 7, a nucleotide sequence having at least 99%, 98%, 97%, 96%, 95%, 90%, 85%, or 80% identity to the nucleotide sequence of nucleotides 53 to 106 of SEQ ID NO: 7, or a fragment of the nucleotide sequence of nucleotides 53 to 106 of SEQ ID NO: 7, or the nucleotide sequence having at least 99%, 98%, 97%, 96%, 95%, 90%, 85%, or 80% identity to the nucleotide sequence of nucleotides 53 to 106 of SEQ ID NO: 7; and/or (ii) encodes an amino acid sequence comprising the amino acid sequence of amino acids 1 to 18 of SEQ ID NO: 6, an amino acid sequence having at least 99%, 98%, 97%, 96%, 95%, 90%, 85%, or 80% identity to the amino acid sequence of amino acids 1 to 18 of SEQ ID NO: 6, or a functional fragment of the amino acid sequence of amino acids 1 to 18 of SEQ ID NO: 6, or the amino acid sequence having at least 99%, 98%, 97%, 96%, 95%, 90%, 85%, or 80% identity to the amino acid sequence of amino acids 1 to 18 of SEQ ID NO: 6. In one embodiment, RNA encoding a signal sequence (i) comprises the nucleotide sequence of nucleotides 53 to 106 of SEQ ID NO: 7; and/or (ii) encodes an amino acid sequence comprising the amino acid sequence of amino acids 1 to 18 of SEQ ID NO: 6.

Such signal peptides are preferably used in order to promote secretion of the encoded polypeptide to which they are fused.

Accordingly, in particularly preferred embodiments, the RNA described herein comprises at least one coding region encoding an immunostimulant protein optionally fused to hAlb and a signal peptide, said signal peptide preferably being fused to immunostimulant protein optionally fused to hAlb, more preferably to the N-terminus of the immunostimulant protein optionally fused to hAlb.

In one embodiment, a hIL7 immunostimulant polypeptide comprises the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In one embodiment, a hIL7 immunostimulant polypeptide comprises the amino acid sequence of SEQ ID NO: 4.

In one embodiment, RNA encoding a hIL7 immunostimulant polypeptide (i) comprises the nucleotide sequence of nucleotides 53 to 2368 of SEQ ID NO: 5, a nucleotide sequence having at least 99%, 98%, 97%, 96%, 95%, 90%, 85%, or 80% identity to the nucleotide sequence of nucleotides 53 to 2368 of SEQ ID NO: 5, or a fragment of the nucleotide sequence of nucleotides 53 to 2368 of SEQ ID NO: 5, or the nucleotide sequence having at least 99%, 98%, 97%, 96%, 95%, 90%, 85%, or 80% identity to the nucleotide sequence of nucleotides 53 to 2368 of SEQ ID NO: 5; and/or (ii) encodes an amino acid sequence comprising the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In one embodiment, RNA encoding a hIL7 immunostimulant polypeptide (i) comprises the nucleotide sequence of nucleotides 53 to 2368 of SEQ ID NO: 5; and/or (ii) encodes an amino acid sequence comprising the amino acid sequence of SEQ ID NO: 4.

In one embodiment, RNA encoding a hIL7 immunostimulant polypeptide (i) comprises the nucleotide sequence of SEQ ID NO: 5, a nucleotide sequence having at least 99%, 98%, 97%, 96%, 95%, 90%, 85%, or 80% identity to the nucleotide sequence of SEQ ID NO: 5, or a fragment of the nucleotide sequence of SEQ ID NO: 5, or the nucleotide sequence having at least 99%, 98%, 97%, 96%, 95%, 90%, 85%, or 80% identity to the nucleotide sequence of SEQ ID NO: 5; and/or (ii) encodes an amino acid sequence comprising the amino acid sequence of SEQ ID NO: 4, an amino acid sequence having at least 99%, 98%, 97%, 96%, 95%, 90%, 85%, or 80% identity to the amino acid sequence of SEQ ID NO: 4, or a functional fragment of the amino acid sequence of SEQ ID NO: 4, or the amino acid sequence having at least 99%, 98%, 97%, 96%, 95%, 90%, 85%, or 80% identity to the amino acid sequence of SEQ ID NO: 4. In one embodiment, RNA encoding a hIL7 immunostimulant polypeptide (i) comprises the nucleotide sequence of SEQ ID NO: 5; and/or (ii) encodes an amino acid sequence comprising the amino acid sequence of SEQ ID NO: 4.

In one embodiment, a hIL2 immunostimulant polypeptide comprises the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In one embodiment, a hIL2 immunostimulant polypeptide comprises the amino acid sequence of SEQ ID NO: 6.

In one embodiment, RNA encoding a hIL2 immunostimulant polypeptide (i) comprises the nucleotide sequence of nucleotides 53 to 2308 of SEQ ID NO: 7, a nucleotide sequence having at least 99%, 98%, 97%, 96%, 95%, 90%, 85%, or 80% identity to the nucleotide sequence of nucleotides 53 to 2308 of SEQ ID NO: 7, or a fragment of the nucleotide sequence of nucleotides 53 to 2308 of SEQ ID NO: 7, or the nucleotide sequence having at least 99%, 98%, 97%, 96%, 95%, 90%, 85%, or 80% identity to the nucleotide sequence of nucleotides 53 to 2308 of SEQ ID NO: 7; and/or (ii) encodes an amino acid sequence comprising the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In one embodiment, RNA encoding a hIL2 immunostimulant polypeptide (i) comprises the nucleotide sequence of nucleotides 53 to 2308 of SEQ ID NO: 7; and/or (ii) encodes an amino acid sequence comprising the amino acid sequence of SEQ ID NO: 6.

In one embodiment, RNA encoding a hIL2 immunostimulant polypeptide (i) comprises the nucleotide sequence of SEQ ID NO: 7, a nucleotide sequence having at least 99%, 98%, 97%, 96%, 95%, 90%, 85%, or 80% identity to the nucleotide sequence of SEQ ID NO: 7, or a fragment of the nucleotide sequence of SEQ ID NO: 7, or the nucleotide sequence having at least 99%, 98%, 97%, 96%, 95%, 90%, 85%, or 80% identity to the nucleotide sequence of SEQ ID NO: 7; and/or (ii) encodes an amino acid sequence comprising the amino acid sequence of SEQ ID NO: 6, an amino acid sequence having at least 99%, 98%, 97%, 96%, 95%, 90%, 85%, or 80% identity to the amino acid sequence of SEQ ID NO: 6, or a functional fragment of the amino acid sequence of SEQ ID NO: 6, or the amino acid sequence having at least 99%, 98%, 97%, 96%, 95%, 90%, 85%, or 80% identity to the amino acid sequence of SEQ ID NO: 6. In one embodiment, RNA encoding a hIL2 immunostimulant polypeptide (i) comprises the nucleotide sequence of SEQ ID NO: 7; and/or (ii) encodes an amino acid sequence comprising the amino acid sequence of SEQ ID NO: 6.

In the following, embodiments of the immunostimulant RNAs are described, wherein certain terms used when describing elements thereof have the following meanings:

hAg-Kozak: 5′-UTR sequence of the human alpha-globin mRNA with an optimized ‘Kozak sequence’ to increase translational efficiency.

SP: Signal peptide. hAlb: Sequences encoding human albumin.

IL2/IL7: Sequences encoding the respective human IL or variant or fragment.

Linker (GS): Sequences coding for linker peptides predominantly consisting of the amino acids glycine (G) and serine (S), as commonly used for fusion proteins.

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency.

In one embodiment, IL7 immunostimulant RNA described herein comprises the structure:
hAgKozak-IL7 with SP-Linker-hAlb mature-FI element-Ligation3-A30LA70 In one embodiment, IL7 immunostimulant described herein comprises the structure:

IL7 with SP-Linker-hAlb mature In one embodiment, IL2 immunostimulant RNA described herein comprises the structure:
hAgKozak-SP-hAlb-Linker-IL2 mature-FI element-Ligation3-A30LA70 In one embodiment, IL2 immunostimulant described herein comprises the structure: SP-hAlb-Linker-IL2 mature

In one embodiment, hAg-Kozak comprises the nucleotide sequence of SEQ ID NO: 13. In one embodiment, IL7 comprises the amino acid sequence of SEQ ID NO: 1. In one embodiment, IL2 comprises the amino acid sequence of SEQ ID NO: 2. In one embodiment, hAlb comprises the amino acid sequence of SEQ ID NO: 3. In one embodiment, Linker comprises the amino acid sequence of SEQ ID NO: 11. In one embodiment, FI comprises the nucleotide sequence of SEQ ID NO: 14. In one embodiment, A30L70 comprises the nucleotide sequence of SEQ ID NO: 15. In one embodiment, the immunostimulant RNAs described herein contain 1-methyl-pseudouridine instead of uridine. The preferred 5′ cap structure is m₂^{7,3′-O}Gppp(m₁^{2′-O})ApG.

RBP009.1 (contained in BNT152)

The nucleotide sequence of RBP009.1 (contained in BNT152), one embodiment of an IL7 immunostimulant RNA, is shown below. In addition, the sequence of the translated protein (hIL7 immunostimulant polypeptide) is shown.

| | |
|---|---|
| agacgaacua guauucuucu gguccccaca gacucagaga gaacccgcca cc aug uuc | 58 |
| Met Phe | |
| 1 | |
| cau guu ucu uuu agg uau auc uuu gga cuu ccu ccc cug auc cuu guu | 106 |
| His Val Ser Phe Arg Tyr Ile Phe Gly Leu Pro Pro Leu Ile Leu Val | |
| 5 10 15 | |
| cug uug cca gua gca uca ucu gau ugu gau auu gaa ggu aaa gau ggc | 154 |
| Leu Leu Pro Val Ala Ser Ser Asp Cys Asp Ile Glu Gly Lys Asp Gly | |
| 20 25 30 | |
| aaa caa uau gag agu guu cua aug guc agc auc gau caa uua uug gac | 202 |
| Lys Gln Tyr Glu Ser Val Leu Met Val Ser Ile Asp Gln Leu Leu Asp | |
| 35 40 45 50 | |
| agc aug aaa gaa auu ggu agc aau ugc cug aau aau gaa uuu aac uuu | 250 |
| Ser Met Lys Glu Ile Gly Ser Asn Cys Leu Asn Asn Glu Phe Asn Phe | |
| 55 60 65 | |
| uuu aaa aga cau auc ugu gau gou aau aag gaa ggu aug uuu uua uuc | 298 |
| Phe Lys Arg His Ile Cys Asp Ala Asn Lys Glu Gly Met Phe Leu Phe | |
| 70 75 80 | |
| cgu gcu gcu cgc aag uug agg caa uuu cuu aaa aug aau agc acu ggu | 346 |
| Arg Ala Ala Arg Lys Leu Arg Gln Phe Leu Lys Met Asn Ser Thr Gly | |
| 85 90 95 | |
| gau uuu gau cuc cac uua uua aaa guu uca gaa ggc aca aca aua cug | 394 |
| Asp Phe Asp Leu His Leu Leu Lys Val Ser Glu Gly Thr Thr Ile Leu | |
| 100 105 110 | |
| uug aac ugc acu ggc cag guu aaa gga aga aaa cca gcu gcc cug ggu | 442 |
| Leu Asn Cys Thr Gly Gln Val Lys Gly Arg Lys Pro Ala Ala Leu Gly | |
| 115 120 125 130 | |
| gaa gcc caa cca aca aag agu uug gaa gaa aau aaa ucu uua aag gaa | 490 |
| Glu Ala Gln Pro Thr Lys Ser Leu Glu Glu Asn Lys Ser Leu Lys Glu | |
| 135 140 145 | |
| cag aaa aaa cug aau gac uug ugu uuc cua aag aga cua uua caa gag | 538 |
| Gln Lys Lys Leu Asn Asp Leu Cys Phe Leu Lys Arg Leu Leu Gln Glu | |
| 150 155 160 | |
| aua aaa acu ugu ugg aau aaa auu uug aug ggc acu aaa gaa cac ggc | 586 |
| Ile Lys Thr Cys Trp Asn Lys Ile Leu Met Gly Thr Lys Glu His Gly | |
| 165 170 175 | |
| ggc ucu gga gga ggc ggc ucc gga ggc gau gca cac aag agu gag guu | 634 |
| Gly Ser Gly Gly Gly Gly Ser Gly Gly Asp Ala His Lys Ser Glu Val | |
| 180 185 190 | |
| gcu cau cgc uuu aaa gau uug gga gaa gaa aau uuc aaa gcc uug gug | 682 |
| Ala His Arg Phe Lys Asp Leu Gly Glu Glu Asn Phe Lys Ala Leu Val | |
| 195 200 205 210 | |
| uug auu gcc uuu gcu cag uau cuu cag cag ugu cca uuu gaa gau cau | 730 |
| Leu Ile Ala Phe Ala Gln Tyr Leu Gln Gln Cys Pro Phe Glu Asp His | |
| 215 220 225 | |
| gua aaa uua gug aau gaa gua acu gaa uuu gca aaa aca ugu guu gcu | 778 |
| Val Lys Leu Val Asn Glu Val Thr Glu Phe Ala Lys Thr Cys Val Ala | |
| 230 235 240 | |
| gau gag uca gcu gaa aau ugu gac aaa uca cuu cau acc cuu uuu gga | 826 |
| Asp Glu Ser Ala Glu Asn Cys Asp Lys Ser Leu His Thr Leu Phe Gly | |
| 245 250 255 | |
| gac aaa uua ugc aca guu gca aca cuu cgu gaa acc uau ggu gaa aug | 874 |
| Asp Lys Leu Cys Thr Val Ala Thr Leu Arg Glu Thr Tyr Gly Glu Met | |
| 260 265 270 | |
| gcu gac ugc ugu gca aaa caa gaa cou gag aga aau gaa ugc uuc uug | 922 |
| Ala Asp Cys Cys Ala Lys Gln Glu Pro Glu Arg Asn Glu Cys Phe Leu | |
| 275 280 285 290 | |
| caa cac aaa gau gac aac cca aac cuc ccc cga uug gug aga cca gag | 970 |
| Gln His Lys Asp Asp Asn Pro Asn Leu Pro Arg Leu Val Arg Pro Glu | |
| 295 300 305 | |
| guu gau gug aug ugc acu gcu uuu cau gac aau gaa gaa aca uuu uug | 1018 |
| Val Asp Val Met Cys Thr Ala Phe His Asp Asn Glu Glu Thr Phe Leu | |
| 310 315 320 | |
| aaa aaa uac uua uau gaa auu gcc aga aga cau ccu uac uuu uau gcc | 1066 |
| Lys Lys Tyr Leu Tyr Glu Ile Ala Arg Arg His Pro Tyr Phe Tyr Ala | |
| 325 330 335 | |
| ccg gaa cuc cuu uuc uuu gcu aaa agg uau aaa gcu gcu uuu aca gaa | 1114 |
| Pro Glu Leu Leu Phe Phe Ala Lys Arg Tyr Lys Ala Ala Phe Thr Glu | |
| 340 345 350 | |
| ugu ugc caa gou gcu gau aaa gcu gcc ugc cug uug cca aag cuc gau | 1162 |
| Cys Cys Gln Ala Ala Asp Lys Ala Ala Cys Leu Leu Pro Lys Leu Asp | |
| 355 360 365 370 | |
| gaa cuu cgg gau gaa ggg aag gcu ucg ucu gcc aaa cag aga cuc aag | 1210 |
| Glu Leu Arg Asp Glu Gly Lys Ala Ser Ser Ala Lys Gln Arg Leu Lys | |
| 375 380 385 | |
| ugu gcc agu cuc caa aaa uuu gga gaa aga gcu uuc aaa gca ugg gca | 1258 |
| Cys Ala Ser Leu Gln Lys Phe Gly Glu Arg Ala Phe Lys Ala Trp Ala | |
| 390 395 400 | |
| gua gcu cgc cug agc cag aga uuu ccc aaa gcu gag uuu gca gaa guu | 1306 |
| Val Ala Arg Leu Ser Gln Arg Phe Pro Lys Ala Glu Phe Ala Glu Val | |
| 405 410 415 | |
| ucc aag uua gug aca gau cuu acc aaa guc cac acg gaa ugc ugc cau | 1354 |
| Ser Lys Leu Val Thr Asp Leu Thr Lys Val His Thr Glu Cys Cys His | |
| 420 425 430 | |
| gga gau cug cuu gaa ugu gcu gau gac agg gcg gac cuu gcc aag uau | 1402 |
| Gly Asp Leu Leu Glu Cys Ala Asp Asp Arg Ala Asp Leu Ala Lys Tyr | |
| 435 440 445 450 | |
| auc ugu gaa aau caa gau ucg auc ucc agu aaa cug aag gaa ugc ugu | 1450 |
| Ile Cys Glu Asn Gln Asp Ser Ile Ser Ser Lys Leu Lys Glu Cys Cys | |
| 455 460 465 | |
| gaa aaa cca cug uug gaa aaa ucc cac ugc auu gcc gaa gug gaa aau | 1498 |
| Glu Lys Pro Leu Leu Glu Lys Ser His Cys Ile Ala Glu Val Glu Asn | |
| 470 475 480 | |
| gau gag aug ccu gcu gac uug ccu uca uua gcu gcu gau uuu guu gaa | 1546 |
| Asp Glu Met Pro Ala Asp Leu Pro Ser Leu Ala Ala Asp Phe Val Glu | |
| 485 490 495 | |
| agu aag gau guu ugc aaa aac uau gou gag gca aag gau guc uuc cug | 1594 |
| Ser Lys Asp Val Cys Lys Asn Tyr Ala Glu Ala Lys Asp Val Phe Leu | |
| 500 505 510 | |
| ggc aug uuu uug uau gaa uau gca aga agg cau ccu gau uac ucu guc | 1642 |
| Gly Met Phe Leu Tyr Glu Tyr Ala Arg Arg His Pro Asp Tyr Ser Val | |
| 515 520 525 530 | |
| gug cug cug cug aga cuu gcc aag aca uau gaa acc acu cua gag aag | 1690 |
| Val Leu Leu Leu Arg Leu Ala Lys Thr Tyr Glu Thr Thr Leu Glu Lys | |
| 535 540 545 | |
| ugc ugu gcc gcu gca gau ccu cau gaa ugc uau gcc aaa gug uuc gau | 1738 |
| Cys Cys Ala Ala Ala Asp Pro His Glu Cys Tyr Ala Lys Val Phe Asp | |
| 550 555 560 | |
| gaa uuu aaa ccu cuu gug gag gag ccu cag aau uua auc aaa caa aau | 1786 |
| Glu Phe Lys Pro Leu Val Glu Glu Pro Gln Asn Leu Ile Lys Gln Asn | |
| 565 570 575 | |
| ugu gag cuu uuu gag cag cuu gga gag uac aaa uuc cag aau gcg cua | 1834 |
| Cys Glu Leu Phe Glu Gln Leu Gly Glu Tyr Lys Phe Gln Asn Ala Leu | |
| 580 585 590 | |
| uua guu cgu uac acc aag aaa gua ccc caa gug uca acu cca acu cuu | 1882 |
| Leu Val Arg Tyr Thr Lys Lys Val Pro Gln Val Ser Thr Pro Thr Leu | |
| 595 600 605 610 | |
| gua gag guc uca aga aac cua gga aaa gug ggc agc aaa ugu ugu aaa | 1930 |
| Val Glu Val Ser Arg Asn Leu Gly Lys Val Gly Ser Lys Cys Cys Lys | |
| 615 620 625 | |
| cau ccu gaa gca aaa aga aug ccc ugu gca gaa gac uau cua ucc gug | 1978 |
| His Pro Glu Ala Lys Arg Met Pro Cys Ala Glu Asp Tyr Leu Ser Val | |
| 630 635 640 | |
| guc cug aac cag uua ugu gug uug cau gag aaa acg cca gua agu gac | 2026 |
| Val Leu Asn Gln Leu Cys Val Leu His Glu Lys Thr Pro Val Ser Asp | |
| 645 650 655 | |
| aga guc acc aaa ugc ugc aca gaa ucc uug gug aac agg cga cca ugc | 2074 |
| Arg Val Thr Lys Cys Cys Thr Glu Ser Leu Val Asn Arg Arg Pro Cys | |
| 660 665 670 | |
| uuu uca gcu cug gaa guc gau gaa aca uac guu ccc aaa gag uuu aau | 2122 |
| Phe Ser Ala Leu Glu Val Asp Glu Thr Tyr Val Pro Lys Glu Phe Asn | |
| 675 680 685 690 | |
| gcu gaa aca uuc acc uuc cau gca gau aua ugc aca cuu ucu gag aag | 2170 |
| Ala Glu Thr Phe Thr Phe His Ala Asp Ile Cys Thr Leu Ser Glu Lys | |
| 695 700 705 | |
| gag aga caa auc aag aaa caa acu gca cuu guu gag cug gug aaa cac | 2218 |
| Glu Arg Gln Ile Lys Lys Gln Thr Ala Leu Val Glu Leu Val Lys His | |
| 710 715 720 | |
| aag ccc aag gca aca aaa gag caa cug aaa gcu guu aug gau gau uuc | 2266 |
| Lys Pro Lys Ala Thr Lys Glu Gln Leu Lys Ala Val Met Asp Asp Phe | |
| 725 730 735 | |
| gca gcu uuu gua gag aag ugc ugc aag gcu gac gau aag gag acc ugc | 2314 |
| Ala Ala Phe Val Glu Lys Cys Cys Lys Ala Asp Asp Lys Glu Thr Cys | |
| 740 745 750 | |
| uuu gcc gag gag ggu aaa aaa cuu guu gcu gca agu caa gcu gcc uua | 2362 |
| Phe Ala Glu Glu Gly Lys Lys Leu Val Ala Ala Ser Gln Ala Ala Leu | |
| 755 760 765 770 | |
| ggc uua uga ugacucgage ugguacugca ugcacgcaau gcuagcugcc | 2411 |
| Gly Leu | |
| ccuuucccgu ccuggguacc ccgagucucc cccgaccucg ggucccaggu augcucccac | 2471 |
| cuccaccugc cccacucacc accucugcua guuccagaca ccucccaagc acgcagcaau | 2531 |
| gcagcucaaa acgcuuagcc uagccacacc cccacgggaa acagcaguga uuaaccuuua | 2591 |
| gcaauaaacg aaaguuuaac uaagcuauac uaaccccagg guuggucaau uucgugccag | 2651 |
| ccacaccgag accuggucca gagucgcuag ccgcgucgcu aaaaaaaaaa aaaaaaaaaa | 2711 |
| aaaaaaaaaa gcauaugacu aaaaaaaaaa aaaaaaaaaa aaaaaaaaaa aaaaaaaaaa | 2771 |
| aaaaaaaaaa aaaaaaaaaa aaaaaaaaaa | 2801 |

RBP006.1 (contained in BNT153)

The nucleotide sequence of RBP006.1 (contained in BNT153), one embodiment of an IL2 immunostimulant RNA, is shown below. In addition, the sequence of the translated protein (hIL2 immunostimulant polypeptide) is shown.

| agacgaacua guauucuucu gguccccaca gacucagaga gaacccgcca cc aug aag | 58 |
|---|---|
| Met Lys | |
| 1 | |
| ugg gua acc uuu auu ucc cuu cuu uuu cuc uuu agc ucg gcu uau ucc | 106 |
| Trp Val Thr Phe Ile Ser Leu Leu Phe Leu Phe Ser Ser Ala Tyr Ser | |
| 5 10 15 | |
| agg ggu gug uuu cgu cga gau gca cac aag agu gag guu gcu cau cgc | 154 |
| Arg Gly Val Phe Arg Arg Asp Ala His Lys Ser Glu Val Ala His Arg | |
| 20 25 30 | |
| uuu aaa gau uug gga gaa gaa aau uuc aaa gcc uug gug uug auu gcc | 202 |
| Phe Lys Asp Leu Gly Glu Glu Asn Phe Lys Ala Leu Val Leu Ile Ala | |
| 35 40 45 50 | |
| uuu gcu cag uau cuu cag cag ugu cca uuu gaa gau cau gua aaa uua | 250 |
| Phe Ala Gln Tyr Leu Gln Gln Cys Pro Phe Glu Asp His Val Lys Leu | |
| 55 60 65 | |
| gug aau gaa gua acu gaa uuu gca aaa aca ugu guu gcu gau gag uca | 298 |
| Val Asn Glu Val Thr Glu Phe Ala Lys Thr Cys Val Ala Asp Glu Ser | |
| 70 75 80 | |
| gcu gaa aau ugu gac aaa uca cuu cau acc cuu uuu gga gac aaa uua | 346 |
| Ala Glu Asn Cys Asp Lys Ser Leu His Thr Leu Phe Gly Asp Lys Leu | |
| 85 90 95 | |
| ugc aca guu gca aca cuu cgu gaa acc uau ggu gaa aug gcu gac ugc | 394 |
| Cys Thr Val Ala Thr Leu Arg Glu Thr Tyr Gly Glu Met Ala Asp Cys | |
| 100 105 110 | |
| ugu gca aaa caa gaa ccu gag aga aau gaa ugc uuc uug caa cac aaa | 442 |
| Cys Ala Lys Gln Glu Pro Glu Arg Asn Glu Cys Phe Leu Gln His Lys | |
| 115 120 125 130 | |
| gau gac aac cca aac cuc ccc cga uug gug aga cca gag guu gau gug | 490 |
| Asp Asp Asn Pro Asn Leu Pro Arg Leu Val Arg Pro Glu Val Asp Val | |
| 135 140 145 | |
| aug ugc acu gcu uuu cau gac aau gaa gaa aca uuu uug aaa aaa uac | 538 |
| Met Cys Thr Ala Phe His Asp Asn Glu Glu Thr Phe Leu Lys Lys Tyr | |
| 150 155 160 | |
| uua uau gaa auu gcc aga aga cau ccu uac uuu uau gcc ccg gaa cuc | 586 |
| Leu Tyr Glu Ile Ala Arg Arg His Pro Tyr Phe Tyr Ala Pro Glu Leu | |
| 165 170 175 | |
| cuu uuc uuu gcu aaa agg uau aaa gcu gcu uuu aca gaa ugu ugc caa | 634 |
| Leu Phe Phe Ala Lys Arg Tyr Lys Ala Ala Phe Thr Glu Cys Cys Gln | |
| 180 185 190 | |
| gcu gcu gau aaa gcu gcc ugc cug uug cca aag cuc gau gaa cuu cgg | 682 |
| Ala Ala Asp Lys Ala Ala Cys Leu Leu Pro Lys Leu Asp Glu Leu Arg | |
| 195 200 205 210 | |
| gau gaa ggg aag gcu ucg ucu gcc aaa cag aga cuc aag ugu gcc agu | 730 |
| Asp Glu Gly Lys Ala Ser Ser Ala Lys Gln Arg Leu Lys Cys Ala Ser | |
| 215 220 225 | |
| cuc caa aaa uuu gga gaa aga gcu uuc aaa gca ugg gca gua gu cgc | 778 |
| Leu Gln Lys Phe Gly Glu Arg Ala Phe Lys Ala Trp Ala Val Ala Arg | |
| 230 235 240 | |
| cug agc cag aga uuu ccc aaa gcu gag uuu gca gaa guu ucc aag uua | 826 |
| Leu Ser Gln Arg Phe Pro Lys Ala Glu Phe Ala Glu Val Ser Lys Leu | |
| 245 250 255 | |
| gug aca gau cuu acc aaa guc cac acg gaa ugc ugc cau gga gau cug | 874 |
| Val Thr Asp Leu Thr Lys Val His Thr Glu Cys Cys His Gly Asp Leu | |
| 260 265 270 | |
| cuu gaa ugu gcu gau gac agg gcg gac cuu gcc aag uau auc ugu gaa | 922 |
| Leu Glu Cys Ala Asp Asp Arg Ala Asp Leu Ala Lys Tyr Ile Cys Glu | |
| 275 280 285 290 | |
| aau caa gau ucg auc ucc agu aaa cug aag gaa ugc ugu gaa aaa cca | 970 |
| Asn Gln Asp Ser Ile Ser Ser Lys Leu Lys Glu Cys Cys Glu Lys Pro | |
| 295 300 305 | |
| cug uug gaa aaa ucc cac ugc auu gcc gaa gug gaa aau gau gag aug | 1018 |
| Leu Leu Glu Lys Ser His Cys Ile Ala Glu Val Glu Asn Asp Glu Met | |
| 310 315 320 | |
| ccu gcu gac uug ccu uca uua gou gou gau uuu guu gaa agu aag gau | 1066 |
| Pro Ala Asp Leu Pro Ser Leu Ala Ala Asp Phe Val Glu Ser Lys Asp | |
| 325 330 335 | |
| guu ugc aaa aac uau gcu gag gca aag gau guc uuc cug ggc aug uuu | 1114 |
| Val Cys Lys Asn Tyr Ala Glu Ala Lys Asp Val Phe Leu Gly Met Phe | |
| 340 345 350 | |
| uug uau gaa uau gca aga agg cau ccu gau uac ucu guc gug cug cug | 1162 |
| Leu Tyr Glu Tyr Ala Arg Arg His Pro Asp Tyr Ser Val Val Leu Leu | |
| 355 360 365 370 | |
| cug aga cuu gcc aag aca uau gaa acc acu cua gag aag ugc ugu gcc | 1210 |
| Leu Arg Leu Ala Lys Thr Tyr Glu Thr Thr Leu Glu Lys Cys Cys Ala | |
| 375 380 385 | |
| gcu gca gau ccu cau gaa ugc uau gcc aaa gug uuc gau gaa uuu aaa | 1258 |
| Ala Ala Asp Pro His Glu Cys Tyr Ala Lys Val Phe Asp Glu Phe Lys | |
| 390 395 400 | |
| ccu cuu gug gag gag ccu cag aau uua auc aaa caa aau ugu gag cuu | 1306 |
| Pro Leu Val Glu Glu Pro Gln Asn Leu Ile Lys Gln Asn Cys Glu Leu | |
| 405 410 415 | |
| uuu gag cag cuu gga gag uac aaa uuc cag aau gcg cua uua guu cgu | 1354 |
| Phe Glu Gln Leu Gly Glu Tyr Lys Phe Gln Asn Ala Leu Leu Val Arg | |
| 420 425 430 | |
| uac acc aag aaa gua ccc caa gug uca acu cca acu cuu gua gag guc | 1402 |
| Tyr Thr Lys Lys Val Pro Gln Val Ser Thr Pro Thr Leu Val Glu Val | |
| 435 440 445 450 | |
| uca aga aac cua gga aaa gug ggc agc aaa ugu ugu aaa cau ccu gaa | 1450 |
| Ser Arg Asn Leu Gly Lys Val Gly Ser Lys Cys Cys Lys His Pro Glu | |
| 455 460 465 | |
| gca aaa aga aug ccc ugu gca gaa gac uau cua ucc gug guc cug aac | 1498 |
| Ala Lys Arg Met Pro Cys Ala Glu Asp Tyr Leu Ser Val Val Leu Asn | |
| 470 475 480 | |
| cag uua ugu gug uug cau gag aaa acg cca gua agu gac aga guc acc | 1546 |
| Gln Leu Cys Val Leu His Glu Lys Thr Pro Val Ser Asp Arg Val Thr | |
| 485 490 495 | |
| aaa ugc ugc aca gaa ucc uug gug aac agg cga cca ugc uuu uca gcu | 1594 |
| Lys Cys Cys Thr Glu Ser Leu Val Asn Arg Arg Pro Cys Phe Ser Ala | |
| 500 505 510 | |
| cug gaa guc gau gaa aca uac guu ccc aaa gag uuu aau gcu gaa aca | 1642 |
| Leu Glu Val Asp Glu Thr Tyr Val Pro Lys Glu Phe Asn Ala Glu Thr | |
| 515 520 525 530 | |
| uuc acc uuc cau gca gau aua ugc aca cuu ucu gag aag gag aga caa | 1690 |
| Phe Thr Phe His Ala Asp Ile Cys Thr Leu Ser Glu Lys Glu Arg Gln | |
| 535 540 545 | |
| auc aag aaa caa acu gca cuu guu gag cug gug aaa cac aag ccc aag | 1738 |
| Ile Lys Lys Gln Thr Ala Leu Val Glu Leu Val Lys His Lys Pro Lys | |
| 550 555 560 | |
| gca aca aaa gag caa cug aaa gcu guu aug gau gau uuc gca gcu uuu | 1786 |
| Ala Thr Lys Glu Gln Leu Lys Ala Val Met Asp Asp Phe Ala Ala Phe | |
| 565 570 575 | |
| gua gag aag ugc ugc aag gcu gac gau aag gag acc ugc uuu gcc gag | 1834 |
| Val Glu Lys Cys Cys Lys Ala Asp Asp Lys Glu Thr Cys Phe Ala Glu | |
| 580 585 590 | |
| gag ggu aaa aaa cuu guu gcu gca agu caa gcu gcc uua ggc uua ggc | 1882 |
| Glu Gly Lys Lys Leu Val Ala Ala Ser Gln Ala Ala Leu Gly Leu Gly | |
| 595 600 605 610 | |
| ggc ucu gga gga ggc ggc ucc gga ggc gcu cca aca ucu ucu uca aca | 1930 |
| Gly Ser Gly Gly Gly Gly Ser Gly Gly Ala Pro Thr Ser Ser Ser Thr | |
| 615 620 625 | |
| aag aaa aca cag cuu cag cuu gaa cac cuu cuu cuu gau cuu cag aug | 1978 |
| Lys Lys Thr Gln Leu Gln Leu Glu His Leu Leu Leu Asp Leu Gln Met | |
| 630 635 640 | |
| auu cug aau gga auc aac aau uac aaa aau cca aaa cug aca aga aug | 2026 |
| Ile Leu Asn Gly Ile Asn Asn Tyr Lys Asn Pro Lys Leu Thr Arg Met | |
| 645 650 655 | |
| cug aca uuu aaa uuu uac aug cca aag aaa gca aca gaa cug aaa cac | 2074 |
| Leu Thr Phe Lys Phe Tyr Met Pro Lys Lys Ala Thr Glu Leu Lys His | |
| 660 665 670 | |
| cuu cag ugc cuu gaa gaa gaa cug aaa ccu cug gaa gaa gug cug aau | 2122 |
| Leu Gln Cys Leu Glu Glu Glu Leu Lys Pro Leu Glu Glu Val Leu Asn | |
| 675 680 685 690 | |
| cug gcu cag agc aaa aau uuu cac cug aga cca aga gau cug auc agc | 2170 |
| Leu Ala Gln Ser Lys Asn Phe His Leu Arg Pro Arg Asp Leu Ile Ser | |
| 695 700 705 | |
| aac auc aau gug auu gug cug gaa cug aaa gga ucu gaa aca aca uuc | 2218 |
| Asn Ile Asn Val Ile Val Leu Glu Leu Lys Gly Ser Glu Thr Thr Phe | |
| 710 715 720 | |
| aug ugu gaa uau gcu gau gaa aca gca aca auu gug gaa uuu cug aac | 2266 |
| Met Cys Glu Tyr Ala Asp Glu Thr Ala Thr Ile Val Glu Phe Leu Asn | |
| 725 730 735 | |
| aga ugg auu aca uuu ugc cag uca auc auu uca aca cug aca uga | 2311 |
| Arg Trp Ile Thr Phe Cys Gln Ser Ile Ile Ser Thr Leu Thr | |
| 740 745 750 | |
| ugacucgage ugguacugca ugcacgcaau gcuagcugcc ccuuucccgu ccuggguacc | 2371 |
| ccgagucucc cccgaccucg ggucccaggu augcucccac cuccaccugc cccacucacc | 2431 |
| accucugcua guuccagaca ccucccaagc acgcagcaau gcagcucaaa acgcuuagcc | 2491 |
| uagccacacc cccacgggaa acagcaguga uuaaccuuua gcaauaaacg aaaguuuaac | 2551 |
| uaagcuauac uaaccccagg guuggucaau uucgugccag ccacaccgag accuggucca | 2611 |
| gagucgcuag ccgcgucgcu aaaaaaaaaa aaaaaaaaaa aaaaaaaaaa gcauaugacu | 2671 |
| aaaaaaaaaa aaaaaaaaaa aaaaaaaaaa aaaaaaaaaa aaaaaaaaaa aaaaaaaaaa | 2731 |
| aaaaaaaaaa | 2741 |

As discussed above, the immunostimulants described herein such as hIL7 immunostimulant or hIL2 immunostimulant are generally present as a fusion protein with an extended-PK group.

The term “fusion protein” as used herein refers to a polypeptide or protein comprising two or more subunits. Preferably, the fusion protein is a translational fusion between the two or more subunits. The translational fusion may be generated by genetically engineering the coding nucleotide sequence for one subunit in a reading frame with the coding nucleotide sequence of a further subunit. Subunits may be interspersed by a linker.

As used herein, the terms “linked,” “fused”, or “fusion” are used interchangeably. These terms refer to the joining together of two or more elements or components or domains.

Immunostimulant polypeptides described herein can be prepared as fusion or chimeric polypeptides that include an immunostimulant portion and a heterologous polypeptide (i.e., a polypeptide that is not an immunostimulant). The immunostimulant may be fused to an extended-PK group, which increases circulation half-life. Non-limiting examples of extended-PK groups are described infra. It should be understood that other PK groups that increase the circulation half-life of immunostimulants such as cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK” immunostimulant refers to an immunostimulant moiety in combination with an extended-PK group. In one embodiment, the extended-PK immunostimulant is a fusion protein in which an immunostimulant moiety is linked or fused to an extended-PK group.

In certain embodiments, the serum half-life of an extended-PK immunostimulant is increased relative to the immunostimulant alone (i.e., the immunostimulant not fused to an extended-PK group). In certain embodiments, the serum half-life of the extended-PK immunostimulant is at least 20, 40, 60, 80, 100, 120, 150, 180, 200, 400, 600, 800, or 1000% longer relative to the serum half-life of the immunostimulant alone. In certain embodiments, the serum half-life of the extended-PK immunostimulant is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the immunostimulant alone. In certain embodiments, the serum half-life of the extended-PK immunostimulant is at least 10 h (h), 15 h, 20 h, 25 h, 30 h, 35 h, 40 h, 50 h, 60 h, 70 h, 80 h, 90 h, 100 h, 110 h, 120 h, 130 h, 135 h, 140 h, 150 h, 160 h, or 200 h.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK immunostimulant suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular an immunostimulant. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3(HSA), i.e., a Fn3 protein that binds to human serum albumin.

In certain aspects, the extended-PK immunostimulant, suitable for use according to the disclosure, can employ one or more peptide linkers. As used herein, the term “peptide linker” refers to a peptide or polypeptide sequence which connects two or more domains (e.g., the extended-PK moiety and an immunostimulant moiety) in a linear amino acid sequence of a polypeptide chain. For example, peptide linkers may be used to connect an immunostimulant moiety to a HSA domain.

Linkers suitable for fusing the extended-PK group to e.g. an immunostimulant are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

### Antigens

The present invention may comprise the use of RNA for vaccination, i.e., the use of RNA encoding an amino acid sequence comprising an antigen, an immunogenic variant thereof, or an immunogenic fragment of the antigen or the immunogenic variant thereof. Thus, the RNA encodes a peptide or protein comprising at least an epitope of an antigen or an immunogenic variant thereof for inducing an immune response against the antigen or cells expressing the antigen in a subject.

The amino acid sequence comprising an antigen, an immunogenic variant thereof, or an immunogenic fragment of the antigen or the immunogenic variant thereof (i.e., the antigenic peptide or protein) is also designated herein as “vaccine antigen”, “peptide and protein antigen”, “antigen molecule” or simply “antigen”. The antigen, an immunogenic variant thereof, or an immunogenic fragment of the antigen or the immunogenic variant thereof is also designated herein as “antigenic peptide or protein” or “antigenic sequence”.

As used herein, the term “vaccine” refers to a composition that induces an immune response upon inoculation into a subject. In some embodiments, the induced immune response provides therapeutic and/or protective immunity.

In one embodiment, the RNA encoding the antigen molecule is expressed in cells of the subject to provide the antigen molecule. In one embodiment, expression of the antigen molecule is at the cell surface or into the extracellular space. In one embodiment, the antigen molecule is presented in the context of MHC. In one embodiment, the RNA encoding the antigen molecule is transiently expressed in cells of the subject. In one embodiment, after administration of the RNA encoding the antigen molecule, expression of the RNA encoding the antigen molecule in spleen occurs. In one embodiment, after administration of the RNA encoding the antigen molecule, expression of the RNA encoding the antigen molecule in antigen presenting cells, preferably professional antigen presenting cells occurs. In one embodiment, the antigen presenting cells are selected from the group consisting of dendritic cells, macrophages and B cells. In one embodiment, after administration of the RNA encoding the antigen molecule, no or essentially no expression of the RNA encoding the antigen molecule in lung and/or liver occurs. In one embodiment, after administration of the RNA encoding the antigen molecule, expression of the RNA encoding the antigen molecule in spleen is at least 5-fold the amount of expression in lung.

The peptide and protein antigens suitable for use according to the disclosure typically include a peptide or protein comprising an epitope of an antigen or a functional variant thereof for inducing an immune response. The peptide or protein or epitope may be derived from a target antigen, i.e. the antigen against which an immune response is to be elicited. For example, the peptide or protein antigen or the epitope contained within the peptide or protein antigen may be a target antigen or a fragment or variant of a target antigen. The target antigen may be a tumor antigen.

The antigen molecule or a procession product thereof, e.g., a fragment thereof, may bind to an antigen receptor such as a BCR or TCR carried by immune effector cells, or to antibodies.

A peptide and protein antigen which may be provided to a subject according to the invention by administering RNA encoding the peptide and protein antigen, i.e., a vaccine antigen, preferably results in the induction of an immune response, e.g., a humoral and/or cellular immune response, and preferably results in stimulation, priming and/or expansion of T cells, in the subject being provided the peptide or protein antigen. Said immune response is preferably directed against a target antigen, in particular a target antigen expressed by diseased cells, tissues and/or organs, i.e., a disease-associated antigen, in particular a tumor antigen. Thus, a vaccine antigen may comprise the target antigen, a variant thereof, or a fragment thereof. In one embodiment, such fragment or variant is immunologically equivalent to the target antigen. In the context of the present disclosure, the term “fragment of an antigen” or “variant of an antigen” means an agent which results in the induction of an immune response and preferably results in stimulation, priming and/or expansion of T cells, which immune response targets the antigen, i.e. a target antigen, in particular when expressed by a target cell and preferably presented in the context of MHC by said target cell. Thus, the vaccine antigen may correspond to or may comprise the target antigen, may correspond to or may comprise a fragment of the target antigen or may correspond to or may comprise an antigen which is homologous to the target antigen or a fragment thereof. Thus, according to the disclosure, a vaccine antigen may comprise an immunogenic fragment of a target antigen or an amino acid sequence being homologous to an immunogenic fragment of a target antigen. An “immunogenic fragment of an antigen” according to the disclosure preferably relates to a fragment of an antigen which is capable of inducing an immune response against the target antigen, in particular a target antigen expressed by diseased cells, tissues and/or organs, i.e., a disease-associated antigen. It is preferred that the vaccine antigen (similar to the target antigen) provides the relevant epitope for binding by T cells. It is also preferred that the vaccine antigen (similar to the target antigen) is presented by a cell such as an antigen-presenting cell and/or diseased cell so as to provide the relevant epitope for binding by the T cells. The vaccine antigen may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence, in particular stimulation, priming and/or expansion of T cells. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. Preferably, clonal expansion leads to differentiation of the immune effector cells.

The term “antigen” relates to an agent comprising an epitope against which an immune response can be generated. The term “antigen” includes, in particular, proteins and peptides. In one embodiment, an antigen is presented by cells of the immune system such as antigen presenting cells like dendritic cells or macrophages. An antigen or a procession product thereof such as a T-cell epitope is in one embodiment bound by a T- or B-cell receptor, or by an immunoglobulin molecule such as an antibody. Accordingly, an antigen or a procession product thereof may react specifically with antibodies or T lymphocytes (T cells). In one embodiment, an antigen is a disease-associated antigen, such as a tumor antigen, and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with cancer, typically tumors. The antigen target may be upregulated during a disease, e.g. infection or cancer. In diseased tissues, antigens can differ from healthy tissue and offer unique possibilities for early detection, specific diagnosis and therapy, especially targeted therapy.

In some embodiments the antigen is a tumor antigen.

In the context of the present invention, the term “tumor antigen” or “tumor-associated antigen” relates to proteins that are expressed or aberrantly expressed in one or more tumor or cancer tissues and preferably are under normal conditions specifically expressed in a limited number of tissues and/or organs or in specific developmental stages, for example, the tumor antigen may be under normal conditions specifically expressed in stomach tissue, preferably in the gastric mucosa, in reproductive organs, e.g., in testis, in trophoblastic tissue, e.g., in placenta, or in germ line cells. In this context, “a limited number” preferably means not more than 3, more preferably not more than 2. The tumor antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. In the context of the present invention, the tumor antigen is preferably associated with the cell surface of a cancer cell and is preferably not or only rarely expressed in normal tissues. Preferably, the tumor antigen or the aberrant expression of the tumor antigen identifies cancer cells. In the context of the present invention, the tumor antigen that is expressed by a cancer cell in a subject, e.g., a patient suffering from a cancer disease, is preferably a self-protein in said subject. In preferred embodiments, the tumor antigen in the context of the present invention is expressed under normal conditions specifically in a tissue or organ that is non-essential, i.e., tissues or organs which when damaged by the immune system do not lead to death of the subject, or in organs or structures of the body which are not or only hardly accessible by the immune system. Preferably, the amino acid sequence of the tumor antigen is identical between the tumor antigen which is expressed in normal tissues and the tumor antigen which is expressed in cancer tissues.

Examples for tumor antigens include p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1,-2,-3, NA88-A, NF1, NY-ESO-1, NY-BR-1, p190 minor BCR-abL, Pm1/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE and WT. Particularly preferred tumor antigens include CLAUDIN-18.2 (CLDN18.2) and CLAUDIN-6 (CLDN6).

The term “viral antigen” refers to any viral component having antigenic properties, i.e. being able to provoke an immune response in an individual.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class Il molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In one embodiment, vaccine antigen is recognized by an immune effector cell such as a T cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In one embodiment, an antigen is presented by a diseased cell such as a cancer cell. In one embodiment, an antigen receptor is a TCR which binds to an epitope of an antigen presented in the context of MHC. In one embodiment, binding of a TCR when expressed by T cells and/or present on T cells to an antigen presented by cells such as antigen presenting cells results in stimulation, priming and/or expansion of said T cells. In one embodiment, binding of a TCR when expressed by T cells and/or present on T cells to an antigen presented on diseased cells results in cytolysis and/or apoptosis of the diseased cells, wherein said T cells preferably release cytotoxic factors, e.g. perforins and granzymes.

The use of multiple epitopes has been shown to promote therapeutic efficacy in tumor vaccine compositions. Such multiple epitopes may be derived from the same or different target antigens and may be present, e.g., as a single polypeptide wherein the epitopes are optionally separated by linkers. For example, cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Rapid sequencing of the tumor mutanome may provide multiple epitopes for individualized vaccines which can be encoded by RNA described herein. In certain embodiments of the present disclosure, the vaccine RNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA that encodes at least five epitopes (termed a “pentatope”) and RNA that encodes at least ten epitopes (termed a “decatope”).

According to certain embodiments, a signal peptide is fused, either directly or through a linker, e.g., a linker having the amino acid sequence according to SEQ ID NO: 11, to an antigen, a variant thereof, or a fragment thereof, i.e., the antigenic peptide or protein (including multi-epitope polypeptides as described above).

Such signal peptides are sequences, which typically exhibit a length of about 15 to 30 amino acids and are preferably located at the N-terminus of the antigenic peptide or protein, without being limited thereto. Signal peptides as defined herein preferably allow the transport of the antigenic peptide or protein as encoded by the RNA into a defined cellular compartment, preferably the cell surface, the endoplasmic reticulum (ER) or the endosomal-lysosomal compartment. In one embodiment, the signal peptide sequence as defined herein includes, without being limited thereto, the signal peptide sequence derived from the sequence encoding the human MHC class I complex (HLA-B51, haplotype A2, B27/B51, Cw2/Cw3), and preferably corresponds to the 78 bp fragment coding for the secretory signal peptide, which guides translocation of the nascent polypeptide chain into the endoplasmatic reticulum, and includes, in particular a sequence comprising the amino acid sequence of SEQ ID NO: 8 or a functional variant thereof.

In one embodiment, a signal sequence comprises the amino acid sequence of SEQ ID NO: 8, an amino acid sequence having at least 99%, 98%, 97%, 96%, 95%, 90%, 85%, or 80% identity to the amino acid sequence of SEQ ID NO: 8, or a functional fragment of the amino acid sequence of SEQ ID NO: 8, or the amino acid sequence having at least 99%, 98%, 97%, 96%, 95%, 90%, 85%, or 80% identity to the amino acid sequence of SEQ ID NO: 8. In one embodiment, a signal sequence comprises the amino acid sequence of SEQ ID NO: 8.

Such signal peptides are preferably used in order to promote secretion of the encoded antigenic peptide or protein. More preferably, a signal peptide as defined herein is fused to an encoded antigenic peptide or protein as defined herein.

Accordingly, in particularly preferred embodiments, the RNA described herein comprises at least one coding region encoding an antigenic peptide or protein and a signal peptide, said signal peptide preferably being fused to the antigenic peptide or protein, more preferably to the N-terminus of the antigenic peptide or protein as described herein.

According to certain embodiments, an amino acid sequence enhancing antigen processing and/or presentation is fused, either directly or through a linker, to an antigen, a variant thereof, or a fragment thereof, i.e., the antigenic peptide or protein.

Such amino acid sequences enhancing antigen processing and/or presentation are preferably located at the C-terminus of the antigenic peptide or protein (and optionally at the C-terminus of an amino acid sequence which breaks immunological tolerance), without being limited thereto. Amino acid sequences enhancing antigen processing and/or presentation as defined herein preferably improve antigen processing and presentation. In one embodiment, the amino acid sequence enhancing antigen processing and/or presentation as defined herein includes, without being limited thereto, sequences derived from the human MHC class I complex (HLA-B51, haplotype A2, B27/B51, Cw2/Cw3), in particular a sequence comprising the amino acid sequence of SEQ ID NO: 9 or a functional variant thereof.

In one embodiment, an amino acid sequence enhancing antigen processing and/or presentation comprises the amino acid sequence of SEQ ID NO: 9, an amino acid sequence having at least 99%, 98%, 97%, 96%, 95%, 90%, 85%, or 80% identity to the amino acid sequence of SEQ ID NO: 9, or a functional fragment of the amino acid sequence of SEQ ID NO: 9, or the amino acid sequence having at least 99%, 98%, 97%, 96%, 95%, 90%, 85%, or 80% identity to the amino acid sequence of SEQ ID NO: 9. In one embodiment, an amino acid sequence enhancing antigen processing and/or presentation comprises the amino acid sequence of SEQ ID NO: 9.

Such amino acid sequences enhancing antigen processing and/or presentation are preferably used in order to promote antigen processing and/or presentation of the encoded antigenic peptide or protein. More preferably, an amino acid sequence enhancing antigen processing and/or presentation as defined herein is fused to an encoded antigenic peptide or protein as defined herein. Accordingly, in particularly preferred embodiments, the RNA described herein comprises at least one coding region encoding an antigenic peptide or protein and an amino acid sequence enhancing antigen processing and/or presentation, said amino acid sequence enhancing antigen processing and/or presentation preferably being fused to the antigenic peptide or protein, more preferably to the C-terminus of the antigenic peptide or protein as described herein.

Amino acid sequences derived from tetanus toxoid of Clostridium tetani may be employed to overcome self-tolerance mechanisms in order to efficiently mount an immune response to self-antigens by providing

T-cell help during priming.

It is known that tetanus toxoid heavy chain includes epitopes that can bind promiscuously to MHC class II alleles and induce CD4⁺ memory T cells in almost all tetanus vaccinated individuals. In addition, the combination of tetanus toxoid (TT) helper epitopes with tumor-associated antigens is known to improve the immune stimulation compared to application of tumor-associated antigen alone by providing CD4⁺-mediated T-cell help during priming. To reduce the risk of stimulating CD8⁺ T cells with the tetanus sequences which might compete with the intended induction of tumor antigen-specific T-cell response, not the whole fragment C of tetanus toxoid is used as it is known to contain CD8⁺ T-cell epitopes. Two peptide sequences containing promiscuously binding helper epitopes were selected alternatively to ensure binding to as many MHC class Il alleles as possible. Based on the data of the ex vivo studies the well-known epitopes p2 (QYIKANSKFIGITEL; TT830-844) and p16 (MTNSVDDALINSTKIYSYFPSVISKVNQGAQG; TT 578-609) were selected. The p2 epitope was already used for peptide vaccination in clinical trials to boost anti-melanoma activity.

Present non-clinical data (unpublished) showed that RNA vaccines encoding both a tumor antigen plus promiscuously binding tetanus toxoid sequences lead to enhanced CD8⁺ T-cell responses directed against the tumor antigen and improved break of tolerance. Immunomonitoring data from patients vaccinated with vaccines including those sequences fused in frame with the tumor antigen-specific sequences reveal that the tetanus sequences chosen are able to induce tetanus-specific T-cell responses in almost all patients.

According to certain embodiments, an amino acid sequence which breaks immunological tolerance is fused, either directly or through a linker, e.g., a linker having the amino acid sequence according to SEQ ID NO: 11, to an antigen, a variant thereof, or a fragment thereof, i.e., the antigenic peptide or protein.

Such amino acid sequences which break immunological tolerance are preferably located at the C-terminus of the antigenic peptide or protein (and optionally at the N-terminus of the amino acid sequence enhancing antigen processing and/or presentation, wherein the amino acid sequence which breaks immunological tolerance and the amino acid sequence enhancing antigen processing and/or presentation may be fused either directly or through a linker, e.g., a linker having the amino acid sequence according to SEQ ID NO: 12), without being limited thereto. Amino acid sequences which break immunological tolerance as defined herein preferably improve T cell responses. In one embodiment, the amino acid sequence which breaks immunological tolerance as defined herein includes, without being limited thereto, sequences derived from tetanus toxoid-derived helper sequences p2 and p16 (P2P16), in particular a sequence comprising the amino acid sequence of SEQ ID NO: 10 or a functional variant thereof.

In one embodiment, an amino acid sequence which breaks immunological tolerance comprises the amino acid sequence of SEQ ID NO: 10, an amino acid sequence having at least 99%, 98%, 97%, 96%, 95%, 90%, 85%, or 80% identity to the amino acid sequence of SEQ ID NO: 10, or a functional fragment of the amino acid sequence of SEQ ID NO: 10, or the amino acid sequence having at least 99%, 98%, 97%, 96%, 95%, 90%, 85%, or 80% identity to the amino acid sequence of SEQ ID NO: 10. In one embodiment, an amino acid sequence which breaks immunological tolerance comprises the amino acid sequence of SEQ ID NO: 10.

Instead of using antigen RNAs fused with tetanus toxoid helper epitope, the antigen-coding RNAs may be co-administered with a separate RNA coding for TT helper epitope during vaccination. Here, the TT helper epitope-coding RNA can be added to each of the antigen-coding RNAs before preparation. In this way, mixed lipoplex nanoparticles may be formed comprising both, antigen and helper epitope coding RNA in order to deliver both compounds to a given APC.

Accordingly, the present invention may provide for the use of particles such as lipoplex particles comprising:
(i) RNA encoding a vaccine antigen, and
(ii) RNA encoding an amino acid sequence which breaks immunological tolerance.

In one embodiment, the amino acid sequence which breaks immunological tolerance comprises helper epitopes, preferably tetanus toxoid-derived helper epitopes.

In one embodiment, the RNA encoding a vaccine antigen is co-formulated as particles such as lipoplex particles with the RNA encoding an amino acid sequence which breaks immunological tolerance at a ratio of about 4:1 to about 16:1, about 6:1 to about 14:1, about 8:1 to about 12:1, or about 10:1.

In the following, embodiments of the vaccine RNAs are described, wherein certain terms used when describing elements thereof have the following meanings:
hAg-Kozak: 5′-UTR sequence of the human alpha-globin mRNA with an optimized ‘Kozak sequence’ to increase translational efficiency.

sec/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain.

Antigen: Sequences encoding the respective antigenic peptide or protein.

Glycine-serine linker (GS): Sequences coding for short linker peptides predominantly consisting of the amino acids glycine (G) and serine (S), as commonly used for fusion proteins.

P2P16: Sequence coding for tetanus toxoid-derived helper epitopes to break immunological tolerance.

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.

A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency.

In one embodiment, vaccine RNA described herein has the structure:
hAg-Kozak-sec-GS(1)-Antigen-GS(2)-P2P16-GS(3)-MITD-FI-A30L70

In one embodiment, vaccine antigen described herein has the structure:
sec-GS(1)-Antigen-GS(2)-P2P16-GS(3)-MITD

In one embodiment, hAg-Kozak comprises the nucleotide sequence of SEQ ID NO: 13. In one embodiment, see comprises the amino acid sequence of SEQ ID NO: 8. In one embodiment, P2P16 comprises the the amino acid sequence of SEQ ID NO: 10. In one embodiment, MITD comprises the the amino acid sequence of SEQ ID NO: 9. In one embodiment, GS(1) comprises the amino acid sequence of SEQ ID NO: 11. In one embodiment, GS(2) comprises the amino acid sequence of SEQ ID NO: 11. In one embodiment, GS(3) comprises the amino acid sequence of SEQ ID NO: 12. In one embodiment, FI comprises the nucleotide sequence of SEQ ID NO: 14. In one embodiment, A30L70 comprises the nucleotide sequence of SEQ ID NO: 15. The preferred 5′ cap structure is beta-S-ARCA(D1).

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA.

The nucleic acids described herein may be recombinant and/or isolated molecules.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a B-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, the RNA is “replicon RNA” or simply a “replicon”, in particular “self-replicating RNA” or “self-amplifying RNA”. In one particularly preferred embodiment, the replicon or self-replicating RNA is derived from or comprises elements derived from a ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In one embodiment, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

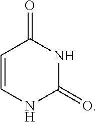

The term “uridine,” as used herein, describes one of the nucleosides that can occur in RNA. The structure of uridine is:

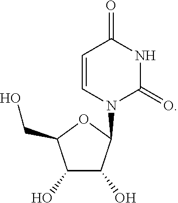 [SMILES: O=c1ccn([C@@H]2O[C@H](CO)[C@@H](O)[C@H]2O)c(=O)[nH]1]

UTP (uridine 5′-triphosphate) has the following structure:

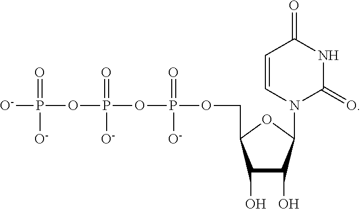

Pseudo-UTP (pseudouridine 5′-triphosphate) has the following structure:

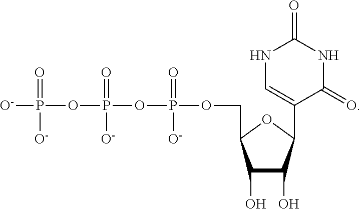

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1\), which has the structure:

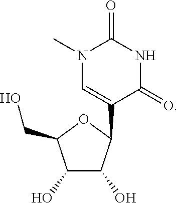 [SMILES: Cn1cc([C@@H]2O[C@H](CO)[C@@H](O)[C@H]2O)c(=O)[nH]c1=O]

N1-methyl-pseudo-UTP has the following structure:

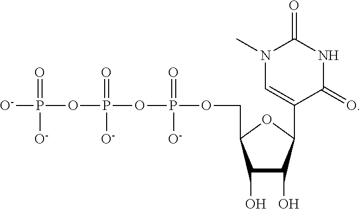

Another exemplary modified nucleoside is 5-methyl-uridine (m5U), which has the structure:

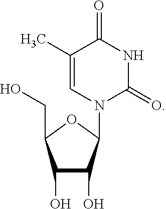

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ) N1-methyl-pseudouridine (m1ψ), and 5-methyl-uridine (m5U). In some embodiments, the modified nucleoside comprises pseudouridine (w). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (w), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl) pseudouridine (acp³ ψ), 5-(isopentenylaminomethyl)uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (m), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In one embodiment, the RNA comprises other modified nucleosides or comprises further modified nucleosides, e.g., modified cytidine. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In one embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′-to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

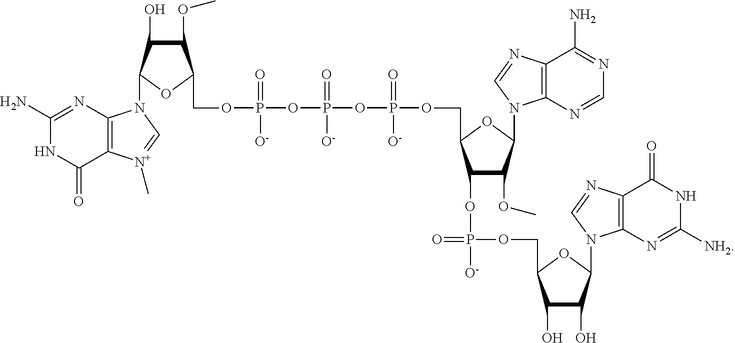

Below is an exemplary Cap1 RNA, which comprises RNA and m₂^{7,3′O}G(5′)ppp(5′)m^{2′-O}ApG:

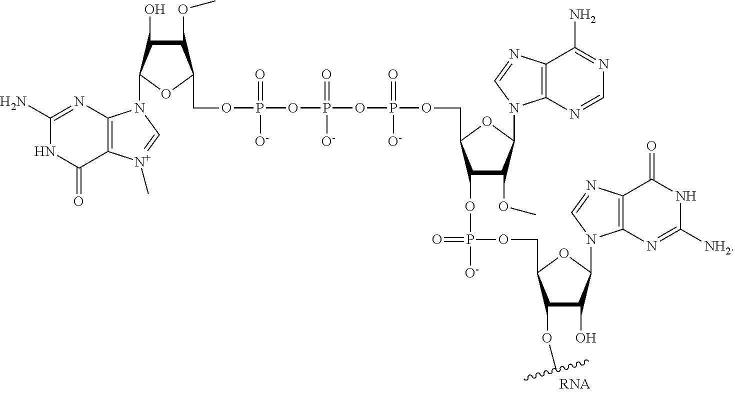

Below is another exemplary Cap1 RNA (no cap analog):

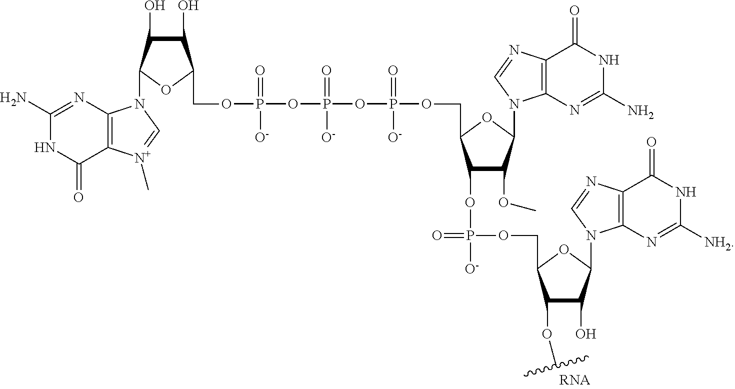

In some embodiments, the RNA is modified with “Cap0” structures using, in one embodiment, the cap analog anti-reverse cap (ARCA Cap (m₂^{7.3′O}G(5′)ppp(5′)G)) with the structure:

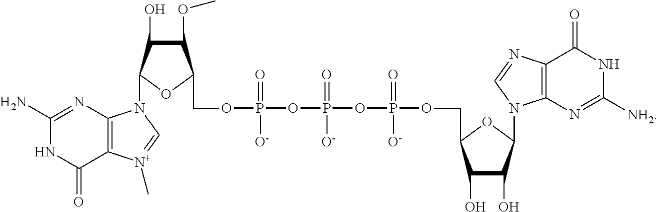

Below is an exemplary Cap0 RNA comprising RNA and m₂^{7,3′O}G(5′)ppp(5′)G:

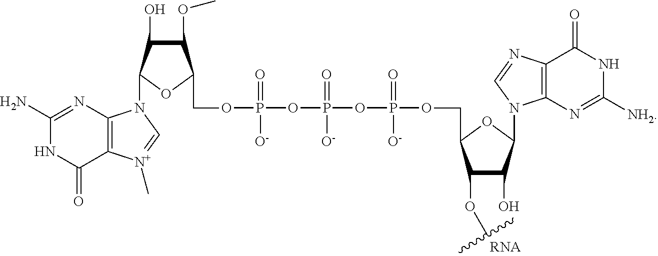

In some embodiments, the “Cap0” structures are generated using the cap analog Beta-S-ARCA (m₂^{7,2′O}G(5′)ppSp(5′)G) with the structure:

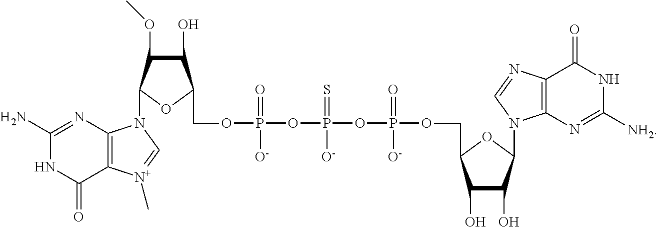

Below is an exemplary Cap0 RNA comprising Beta-S-ARCA (m₂^{7,2′O}G(5′)ppSp(5′)G) and RNA:

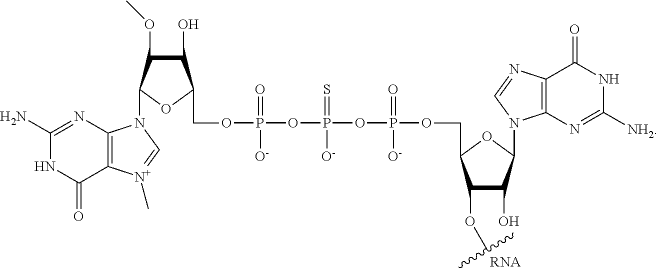

The “D1” diastereomer of beta-S-ARCA or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time (cf., WO 2011/015347, herein incorporated by reference).

A particularly preferred cap is beta-S-ARCA(D1) (m₂^{7,2′-O}GppSpG) or m₂^{7,3′-O}Gppp(m₁^{2′-O})ApG. In one embodiment, in the case of RNA encoding an immostimulant, a preferred cap is m₂^{7,3′-O}Gppp(m₁^{2′-O})ApG. In one embodiment, in the case of RNA encoding a vaccine antigen, a preferred cap is beta-S-ARCA(D1) (m₂^{7,2′-O}GppSpG).

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, RNA comprises a 5′-UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

In some embodiments, RNA comprises a 3′-UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

A particularly preferred 5′-UTR comprises the nucleotide sequence of SEQ ID NO: 13. A particularly preferred 3′-UTR comprises the nucleotide sequence of SEQ ID NO: 14.

In some embodiments, the RNA according to the present disclosure comprises a 3′-poly(A) sequence.

As used herein, the term “poly(A) sequence” or “poly-A tail” refers to an uninterrupted or interrupted sequence of adenylate residues which is typically located at the 3′-end of an RNA molecule. Poly(A) sequences are known to those of skill in the art and may follow the 3′-UTR in the RNAs described herein.

An uninterrupted poly(A) sequence is characterized by consecutive adenylate residues. In nature, an uninterrupted poly(A) sequence is typical. RNAs disclosed herein can have a poly(A) sequence attached to the free 3′-end of the RNA by a template-independent RNA polymerase after transcription or a poly(A) sequence encoded by DNA and transcribed by a template-dependent RNA polymerase.

It has been demonstrated that a poly(A) sequence of about 120 A-nucleotides has a beneficial influence on the levels of RNA in transfected eukaryotic cells, as well as on the levels of protein that is translated from an open reading frame that is present upstream (5′) of the poly(A) sequence (Holtkamp et al., 2006, Blood, vol. 108, pp. 4009-4017).

The poly(A) sequence may be of any length. In some embodiments, a poly(A) sequence comprises, essentially consists of, or consists of at least 20, at least 30, at least 40, at least 80, or at least 100 and up to 500, up to 400, up to 300, up to 200, or up to 150 A-nucleotides, and, in particular, about 120 A-nucleotides. In this context, “essentially consists of” means that most nucleotides in the poly(A) sequence, typically at least 75%, at least 80%, at least 85%, at least 90%, at least 95%, at least 96%, at least 97%, at least 98%, or at least 99% by number of nucleotides in the poly(A) sequence are A nucleotides, but permits that remaining nucleotides are nucleotides other than A-nucleotides, such as U nucleotides (uridylate), G nucleotides (guanylate), or C nucleotides (cytidylate). In this context, “consists of” means that all nucleotides in the poly(A) sequence, i.e., 100% by number of nucleotides in the poly(A) sequence, are A-nucleotides. The term “A nucleotide” or “A” refers to adenylate.

In some embodiments, a poly(A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly(A) sequence contained in an RNA molecule described herein essentially consists of A-nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly(A) sequence at its 3′-end, i.e., the poly(A) sequence is not masked or followed at its 3′-end by a nucleotide other than A.

In some embodiments, the poly(A) sequence may comprise at least 20, at least 30, at least 40, at least 80, or at least 100 and up to 500, up to 400, up to 300, up to 200, or up to 150 nucleotides. In some embodiments, the poly(A) sequence may essentially consist of at least 20, at least 30, at least 40, at least 80, or at least 100 and up to 500, up to 400, up to 300, up to 200, or up to 150 nucleotides. In some embodiments, the poly(A) sequence may consist of at least 20, at least 30, at least 40, at least 80, or at least 100 and up to 500, up to 400, up to 300, up to 200, or up to 150 nucleotides. In some embodiments, the poly(A) sequence comprises at least 100 nucleotides. In some embodiments, the poly(A) sequence comprises about 150 nucleotides. In some embodiments, the poly(A) sequence comprises about 120 nucleotides.

In some embodiments, RNA comprises a poly(A) sequence comprising the nucleotide sequence of SEQ ID NO: 15, or a nucleotide sequence having at least 99%, 98%, 97%, 96%, 95%, 90%, 85%, or 80% identity to the nucleotide sequence of SEQ ID NO: 15.

A particularly preferred poly(A) sequence comprises the nucleotide sequence of SEQ ID NO: 15.

According to the disclosure, RNA is preferably administered as single-stranded, 5′-capped mRNA that is translated into the respective protein upon entering cells of a subject being administered the RNA. Preferably, the RNA contains structural elements optimized for maximal efficacy of the RNA with respect to stability and translational efficiency (5′-cap, 5′-UTR, 3′-UTR, poly(A) sequence).

In one embodiment, after administration of the RNA described herein, e.g., formulated as RNA lipid particles, at least a portion of the RNA is delivered to cells of the subject treated. In one embodiment, at least a portion of the RNA is delivered to the cytosol of the cells. In one embodiment, the RNA is translated by the cells to produce the peptide or protein it encodes. In one embodiment of all aspects of the invention, the RNA is transiently expressed in cells of the subject. In one embodiment of all aspects of the invention, the RNA is in vitro transcribed RNA. In one embodiment of all aspects of the invention, in the case of RNA encoding an immunostimulant, the cells are liver cells. In one embodiment, expression of the immunostimulant is into the extracellular space, i.e., the immunostimulant is secreted. In one embodiment of all aspects of the invention, in the case of RNA encoding a vaccine antigen, the cells are spleen cells. In one embodiment of all aspects of the invention, in the case of RNA encoding a vaccine antigen, the cells are antigen presenting cells such as professional antigen presenting cells in the spleen. In one embodiment, the cells are dendritic cells or macrophages. In one embodiment, the vaccine antigen is expressed and presented in the context of MHC. RNA particles such as RNA lipid particles described herein may be used for delivering RNA to such cells. For example, lipid nanoparticles (LNP) as described herein may be used for delivering RNA encoding an immunostimulant to liver. For example, lipoplex particles (LPX) as described herein may be used for delivering RNA encoding a vaccine antigen to spleen. In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts. Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

The term “expression” as used herein is defined as the transcription and/or translation of a particular nucleotide sequence.

With respect to RNA, the term “expression” or “translation” relates to the process in the ribosomes of a cell by which a strand of mRNA directs the assembly of a sequence of amino acids to make a peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In one embodiment, the RNA to be administered according to the invention is non-immunogenic.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In one preferred embodiment, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In one embodiment, the modified nucleosides comprise a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m⁵Um), 2′-O-methyl-pseudouridine (4m), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m¹ψ) or 5-methyl-uridine (m⁵U), in particular N1-methyl-pseudouridine.

In one embodiment, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaselll that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In one embodiment, the removal of dsRNA from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In one embodiment, the non-immunogenic RNA is free or essentially free of dsRNA. In some embodiments, the non-immunogenic RNA composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In one embodiment, the non-immunogenic RNA is translated in a cell more efficiently than standard RNA with the same sequence. In one embodiment, translation is enhanced by a factor of 2-fold relative to its unmodified counterpart. In one embodiment, translation is enhanced by a 3-fold factor. In one embodiment, translation is enhanced by a 4-fold factor. In one embodiment, translation is enhanced by a 5-fold factor. In one embodiment, translation is enhanced by a 6-fold factor. In one embodiment, translation is enhanced by a 7-fold factor. In one embodiment, translation is enhanced by an 8-fold factor.

In one embodiment, translation is enhanced by a 9-fold factor. In one embodiment, translation is enhanced by a 10-fold factor. In one embodiment, translation is enhanced by a 15-fold factor. In one embodiment, translation is enhanced by a 20-fold factor. In one embodiment, translation is enhanced by a 50-fold factor. In one embodiment, translation is enhanced by a 100-fold factor. In one embodiment, translation is enhanced by a 200-fold factor. In one embodiment, translation is enhanced by a 500-fold factor. In one embodiment, translation is enhanced by a 1000-fold factor. In one embodiment, translation is enhanced by a 2000-fold factor. In one embodiment, the factor is 10-1000-fold. In one embodiment, the factor is 10-100-fold. In one embodiment, the factor is 10-200-fold. In one embodiment, the factor is 10-300-fold. In one embodiment, the factor is 10-500-fold. In one embodiment, the factor is 20-1000-fold. In one embodiment, the factor is 30-1000-fold. In one embodiment, the factor is 50-1000-fold. In one embodiment, the factor is 100-1000-fold. In one embodiment, the factor is 200-1000-fold. In one embodiment, translation is enhanced by any other significant amount or range of amounts.

In one embodiment, the non-immunogenic RNA exhibits significantly less innate immunogenicity than standard RNA with the same sequence. In one embodiment, the non-immunogenic RNA exhibits an innate immune response that is 2-fold less than its unmodified counterpart. In one embodiment, innate immunogenicity is reduced by a 3-fold factor. In one embodiment, innate immunogenicity is reduced by a 4-fold factor. In one embodiment, innate immunogenicity is reduced by a 5-fold factor. In one embodiment, innate immunogenicity is reduced by a 6-fold factor. In one embodiment, innate immunogenicity is reduced by a 7-fold factor. In one embodiment, innate immunogenicity is reduced by an 8-fold factor. In one embodiment, innate immunogenicity is reduced by a 9-fold factor. In one embodiment, innate immunogenicity is reduced by a 10-fold factor. In one embodiment, innate immunogenicity is reduced by a 15-fold factor. In one embodiment, innate immunogenicity is reduced by a 20-fold factor. In one embodiment, innate immunogenicity is reduced by a 50-fold factor. In one embodiment, innate immunogenicity is reduced by a 100-fold factor. In one embodiment, innate immunogenicity is reduced by a 200-fold factor. In one embodiment, innate immunogenicity is reduced by a 500-fold factor. In one embodiment, innate immunogenicity is reduced by a 1000-fold factor. In one embodiment, innate immunogenicity is reduced by a 2000-fold factor.

The term “exhibits significantly less innate immunogenicity” refers to a detectable decrease in innate immunogenicity. In one embodiment, the term refers to a decrease such that an effective amount of the non-immunogenic RNA can be administered without triggering a detectable innate immune response. In one embodiment, the term refers to a decrease such that the non-immunogenic RNA can be repeatedly administered without eliciting an innate immune response sufficient to detectably reduce production of the protein encoded by the non-immunogenic RNA. In one embodiment, the decrease is such that the non-immunogenic RNA can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

### Codon-Optimization/Increase in G/C Content

In some embodiment, an amino acid sequence described herein is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present invention, coding regions are preferably codon-optimized for optimal expression in a subject to be treated using the RNA molecules described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments of the invention, the guanosine/cytosine (G/C) content of the coding region of the RNA described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA described herein is increased by at least 10%, at least 20%, at least 30%, at least 40%, at least 50%, at least 55%, or even more compared to the G/C content of the coding region of the wild type RNA.

### Nucleic Acid Containing Particles

Nucleic acids such as RNA described herein may be administered formulated as particles.

In the context of the present disclosure, the term “particle” relates to a structured entity formed by molecules or molecule complexes. In one embodiment, the term “particle” relates to a micro- or nano-sized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and/or the cationic polymer combine together with the nucleic acid to form aggregates, and this aggregation results in colloidally stable particles.

In one embodiment, particles described herein further comprise at least one lipid or lipid-like material other than a cationic or cationically ionizable lipid or lipid-like material, at least one polymer other than a cationic polymer, or a mixture thereof

In some embodiments, nucleic acid particles comprise more than one type of nucleic acid molecules, where the molecular parameters of the nucleic acid molecules may be similar or different from each other, like with respect to molar mass or fundamental structural elements such as molecular architecture, capping, coding regions or other features,

Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

With respect to RNA lipid particles, the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included. Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

LNPs typically comprise four components: ionizable cationic lipids, neutral lipids such as phospholipids, a steroid such as cholesterol, and a polymer conjugated lipid such as polyethylene glycol (PEG)-lipids. Each component is responsible for payload protection, and enables effective intracellular delivery. LNPs may be prepared by mixing lipids dissolved in ethanol rapidly with nucleic acid in an aqueous buffer.

The term “average diameter” refers to the mean hydrodynamic diameter of particles as measured by dynamic laser light scattering (DLS) with data analysis using the so-called cumulant algorithm, which provides as results the so-called Zaverage with the dimension of a length, and the polydispersity index (PI), which is dimensionless (Koppel, D., J. Chem. Phys. 57, 1972, U.S. Plant Pat. No. 4,814-4820, ISO 13321). Here “average diameter”, “diameter” or “size” for particles is used synonymously with this value of the Zaverage.

The “polydispersity index” is preferably calculated based on dynamic light scattering measurements by the so-called cumulant analysis as mentioned in the definition of the “average diameter”. Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle.

The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In particulate formulation, it is possible that each RNA species (e.g. RNA encoding hIL7 immunostimulant and RNA encoding hIL2 immunostimulant) is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In one embodiment, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, as described above, followed by a step of mixing of the individual particulate formulations. By the step of mixing, a formulation comprising a mixed population of RNA-containing particles is obtainable (for illustration: e.g. a first population of particles may contain RNA encoding hIL7 immunostimulant, and a second formulation of particles may contain RNA encoding hIL2 immunostimulant). Individual particulate populations may be together in one container, comprising a mixed population of individual particulate formulations. Alternatively, it is possible that all RNA species of the pharmaceutical composition (e.g. RNA encoding hIL7 immunostimulant and RNA encoding hIL2 immunostimulant) are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Cationic Polymer

Given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(ß-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties such as those described herein.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine, in particular protamine. The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75.102 to 107 Da, preferably 1000 to 10⁵ Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

Lipid and lipid-like material

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids.

In certain embodiments, the amphiphilic compound is a lipid. The term “lipid” refers to a group of organic compounds that are characterized by being insoluble in water, but soluble in many organic solvents. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides. Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

Cationic or cationically ionizable lipids or lipid-like materials

The nucleic acid particles described herein may comprise at least one cationic or cationically ionizable lipid or lipid-like material as particle forming agent. Cationic or cationically ionizable lipids or lipid-like materials contemplated for use herein include any cationic or cationically ionizable lipids or lipid-like materials which are able to electrostatically bind nucleic acid. In one embodiment, cationic or cationically ionizable lipids or lipid-like materials contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

For purposes of the present disclosure, such “cationically ionizable” lipids or lipid-like materials are comprised by the term “cationic lipid or lipid-like material” unless contradicted by the circumstances.

In one embodiment, the cationic or cationically ionizable lipid or lipid-like material comprises a head group which includes at least one nitrogen atom (N) which is positive charged or capable of being protonated.

Examples of cationic lipids include, but are not limited to 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N-(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide) ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis, cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N, N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N, N-dimethyl-2,3-bis(tetradecyloxy)-1-propanaminium bromide (DMRIE), (+)-N-(3-aminopropyl)-N,N-dimethyl-2,3-bis(cis-9-tetradecenyloxy)-1-propanaminium bromide (GAP-DMORIE), (+)-N-(3-aminopropyl)-N,N-dimethyl-2,3-bis(dodecyloxy)-1-propanaminium bromide (GAP-DLRIE), (+)-N-(3-aminopropyl)-N,N-dimethyl-2,3-bis(tetradecyloxy)-1-propanaminium bromide

(GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(oleoyloxy)propan-1-aminium (DOBAQ), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle.

Additional lipids or lipid-like materials

Particles described herein may also comprise lipids or lipid-like materials other than cationic or cationically ionizable lipids or lipid-like materials, i.e., non-cationic lipids or lipid-like materials (including non-cationically ionizable lipids or lipid-like materials). Collectively, anionic and neutral lipids or lipid-like materials are referred to herein as non-cationic lipids or lipid-like materials. Optimizing the formulation of nucleic acid particles by addition of other hydrophobic moieties, such as cholesterol and lipids, in addition to an ionizable/cationic lipid or lipid-like material may enhance particle stability and efficacy of nucleic acid delivery.

An additional lipid or lipid-like material may be incorporated which may or may not affect the overall charge of the nucleic acid particles. In certain embodiments, the additional lipid or lipid-like material is a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH. In preferred embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain preferred embodiments, the additional lipid is DSPC or DSPC and cholesterol.

In certain embodiments, the nucleic acid particles include both a cationic lipid and an additional lipid.

In one embodiment, particles described herein include a polymer conjugated lipid such as a pegylated lipid. The term “pegylated lipid” refers to a molecule comprising both a lipid portion and a polyethylene glycol portion. Pegylated lipids are known in the art.

Without wishing to be bound by theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some embodiments, the molar ratio of the at least one cationic lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle.

### Lipoplex Particles

In certain embodiments of the present disclosure, the RNA described herein may be present in RNA lipoplex particles.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex s comprise 1,2-di-O-octadecenyl-3-trimethylammonium propane (DOTMA) and 1,2-di-(9Z-octadecenoyl)-sn-glycero-3-phosphoethanolamine (DOPE).

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

Lipid nanoparticles (LNPs)

In one embodiment, nucleic acid such as RNA described herein is present in the form of lipid nanoparticles (LNPs). The LNP may comprise any lipid capable of forming a particle to which the one or more nucleic acid molecules are attached, or in which the one or more nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid, a neutral lipid, a steroid, a polymer conjugated lipid; and the RNA, encapsulated within or associated with the lipid nanoparticle.

In one embodiment, the LNP comprises from 40 to 60 mol percent, or from 50 to 60 mol percent of the cationic lipid.

In one embodiment, the neutral lipid is present in a concentration ranging from 5 to 15 mol percent, from 7 to 13 mol percent, or from 9 to 12 mol percent.

In one embodiment, the steroid is present in a concentration ranging from 30 to 50 mol percent, or from 30 to 40 mol percent.

In one embodiment, the LNP comprises from 1 to 10 mol percent, from 1 to 5 mol percent, or from 1 to 2.5 mol percent of the polymer conjugated lipid.

In one embodiment, the LNP comprises from 40 to 60 mol percent a cationic lipid; from 5 to 15 mol percent of a neutral lipid; from 30 to 50 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure:

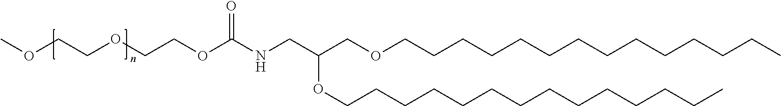

wherein n has a mean value ranging from 30 to 60, such as about 50. In one embodiment, the pegylated lipid is PEG₂₀₀₀-C-DMA.

In one embodiment, the cationic lipid component of the LNPs has the following structure:

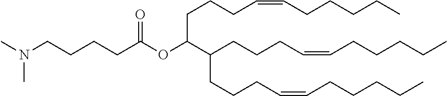 [SMILES: CCCCC/C=C\CCCC(CCC/C=C\CCCCC)C(CCC/C=C\CCCCC)OC(=O)CCCCN(C)C]

In one embodiment, the cationic lipid is 3D-P-DMA.

In some embodiments, the LNP comprises 3D-P-DMA, RNA, a neutral lipid, a steroid and a pegylated lipid. In some embodiments, the neutral lipid is DSPC. In some embodiments, the steroid is cholesterol. In some embodiments, the pegylated lipid is PEG₂₀₀₀-C-DMA.

In some embodiments, the 3D-P-DMA is present in the LNP in an amount from about 40 to about 60 mole percent. In one embodiment, the neutral lipid is present in the LNP in an amount from about 5 to about 15 mole percent. In one embodiment, the steroid is present in the LNP in an amount from about 30 to about 50 mole percent. In one embodiment, the pegylated lipid such as PEG₂₀₀₀-C-DMA is present in the LNP in an amount from about 1 to about 10 mole percent.

### RNA Targeting

Some aspects of the disclosure involve the targeted delivery of the RNA disclosed herein (e.g., RNA encoding immunostimulants or RNA encoding vaccine antigens).

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2, or about 1.6:2 to about 1:2, or about 1.6:2 to about 1.1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

Immunostimulants such as hIL7 and/or hIL2 may be provided to a subject by administering to the subject RNA encoding an immunostimulant in a formulation for preferential delivery of RNA to liver or liver tissue. The delivery of RNA to such target organ or tissue is preferred, in particular, if it is desired to express large amounts of the immunostimulant and/or if systemic presence of the immunostimulant, in particular in significant amounts, is desired or required.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses. Furthermore, lipid nanoparticles (LNPs) as described herein may be used to transport RNA into the liver.

### Immune Checkpoint Inhibitor

As described herein, in one embodiment, the RNA described herein such as immunostimulant RNA and optionally vaccine RNA is administered together, i.e., co-administered, with a checkpoint inhibitor to a subject, e.g., a patient. In certain embodiments, the checkpoint inhibitor and the RNA are administered as a single composition to the subject. In certain embodiments, the checkpoint inhibitor and the RNA are administered concurrently (as separate compositions at the same time) to the subject. In certain embodiments, the checkpoint inhibitor and the RNA are administered separately to the subject. In certain embodiments, the checkpoint inhibitor is administered before the RNA to the subject. In certain embodiments, the checkpoint inhibitor is administered after the RNA to the subject. In certain embodiments, the checkpoint inhibitor and the RNA are administered to the subject on the same day. In certain embodiments, the checkpoint inhibitor and the RNA are administered to the subject on different days.

As used herein, “immune checkpoint” refers to regulators of the immune system, and, in particular, co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2. In certain embodiments, the inhibitory signal is the interaction between CTLA-4 and CD80 or CD86 to displace CD28 binding. In certain embodiments the inhibitory signal is the interaction between LAG-3 and MHC class Il molecules. In certain embodiments, the inhibitory signal is the interaction between TIM-3 and one or more of its ligands, such as galectin 9, PtdSer, HMGB1 and CEACAM1. In certain embodiments, the inhibitory signal is the interaction between one or several KIRs and their ligands. In certain embodiments, the inhibitory signal is the interaction between TIGIT and one or more of its ligands, PVR, PVRL2 and PVRL3. In certain embodiments, the inhibitory signal is the interaction between CD94/NKG2A and HLA-E. In certain embodiments, the inhibitory signal is the interaction between VISTA and its binding partner(s). In certain embodiments, the inhibitory signal is the interaction between one or more Siglecs and their ligands. In certain embodiments, the inhibitory signal is the interaction between GARP and one or more of it ligands. In certain embodiments, the inhibitory signal is the interaction between CD47 and SIRPa. In certain embodiments, the inhibitory signal is the interaction between PVRIG and PVRL2. In certain embodiments, the inhibitory signal is the interaction between CSF1R and CSF1. In certain embodiments, the inhibitory signal is the interaction between BTLA and HVEM. In certain embodiments, the inhibitory signal is part of the adenosinergic pathway, e.g., the interaction between A2AR and/or A2BR and adenosine, produced by CD39 and CD73. In certain embodiments, the inhibitory signal is the interaction between B7-H3 and its receptor and/or B7-H4 and its receptor. In certain embodiments, the inhibitory signal is mediated by IDO, CD20, NOX or TDO.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 (also known as B7-H1 or CD274) and PD-L2 (also known as B7-DC or CD273). The term “PD-1” as used herein includes human PD-1 (hPD-1), variants, isoforms, and species homologs of hPD-1, and analogs having at least one common epitope with hPD-1. “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 what results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

“Cytotoxic T Lymphocyte Associated Antigen-4 (CTLA-4)” (also known as CD152) is a T cell surface molecule and is a member of the immunoglobulin superfamily. This protein downregulates the immune system by binding to CD80 (B7-1) and CD86 (B7-2). The term “CTLA-4” as used herein includes human CTLA-4 (hCTLA-4), variants, isoforms, and species homologs of hCTLA-4, and analogs having at least one common epitope with hCTLA-4. CTLA-4 is a homolog of the stimulatory checkpoint protein CD28 with much higher binding affinity for CD80 and CD86. CTLA4 is expressed on the surface of activated T cells and its ligands are expressed on the surface of professional antigen-presenting cells. Binding of CTLA-4 to its ligands prevents the co-stimulatory signal of CD28 and produces an inhibitory signal. Thus, CTLA-4 downregulates T cell activation.

“T cell Immunoreceptor with Ig and ITIM domains” (TIGIT, also known as WUCAM or Vstm3) is an immune receptor on T cells and Natural Killer (NK) cells and binds to PVR (CD155) on DCs, macrophages etc., and PVRL2 (CD112; nectin-2) and PVRL3 (CD113; nectin-3) and regulates T cell-mediated immunity. The term “TIGIT” as used herein includes human TIGIT (hTIGIT), variants, isoforms, and species homologs of hTIGIT, and analogs having at least one common epitope with hTIGIT. The term “PVR” as used herein includes human PVR (hPVR), variants, isoforms, and species homologs of hPVR, and analogs having at least one common epitope with hPVR. The term “PVRL2” as used herein includes human PVRL2 (hPVRL2), variants, isoforms, and species homologs of hPVRL2, and analogs having at least one common epitope with hPVRL2. The term “PVRL3” as used herein includes human PVRL3 (hPVRL3), variants, isoforms, and species homologs of hPVRL3, and analogs having at least one common epitope with hPVRL3.

The “B7 family” refers to inhibitory ligands with undefined receptors. The B7 family encompasses B7-H3 and B7-H4, both upregulated on tumor cells and tumor infiltrating cells. The terms “B7-H3” and “B7-H4” as used herein include human B7-H3 (hB7-H3) and human B7-H4 (hB7-H4), variants, isoforms, and species homologs thereof, and analogs having at least one common epitope with B7-H3 and B7-H4, respectively. “B and T Lymphocyte Attenuator” (BTLA, also known as CD272) is a TNFR family member expressed in Th1 but not Th2 cells. BTLA expression is induced during activation of T cells and is in particular expressed on surfaces of CD8⁺ T cells. The term “BTLA” as used herein includes human BTLA (hBTLA), variants, isoforms, and species homologs of hBTLA, and analogs having at least one common epitope with hBTLA. BTLA expression is gradually downregulated during differentiation of human CD8⁺ T cells to effector cell phenotype. Tumor-specific human CD8⁺ T cells express high levels of BTLA. BTLA binds to “Herpesvirus entry mediator” (HVEM, also known as TNFRSF14 or CD270) and is involved in T cell inhibition. The term “HVEM” as used herein includes human HVEM (hHVEM), variants, isoforms, and species homologs of hHVEM, and analogs having at least one common epitope with hHVEM. BTLA-HVEM complexes negatively regulate T cell immune responses.

“Killer-cell Immunoglobulin-like Receptors” (KIRs) are receptors for MHC Class I molecules on NK T cells and NK cells that are involved in differentiation between healthy and diseased cells. KIRs bind to human leukocyte antigen (HLA) A, B and C, what suppresses normal immune cell activation. The term “KIRs” as used herein includes human KIRs (hKIRs), variants, isoforms, and species homologs of hKIRs, and analogs having at least one common epitope with a hKIR. The term “HLA” as used herein includes variants, isoforms, and species homologs of HLA, and analogs having at least one common epitope with a HLA. KIR as used herein in particular refers to KIR2DL1, KIR2DL2, and/or KIR2DL3.

“Lymphocyte Activation Gene-3 (LAG-3)” (also known as CD223) is an inhibitory receptor associated with inhibition of lymphocyte activity by binding to MHC class Il molecules. This receptor enhances the function of Treg cells and inhibits CD8⁺ effector T cell function leading to immune response suppression. LAG-3 is expressed on activated T cells, NK cells, B cells and DCs. The term “LAG-3” as used herein includes human LAG-3 (hLAG-3), variants, isoforms, and species homologs of hLAG-3, and analogs having at least one common epitope.

“T cell Membrane Protein-3 (TIM-3)” (also known as HAVcr-2) is an inhibitory receptor involved in the inhibition of lymphocyte activity by inhibition of Th1 cell responses. Its ligand is galectin 9 (GAL9), which is upregulated in various types of cancers. Other TIM-3 ligands include phosphatidyl serine (PtdSer), High Mobility Group Protein 1 (HMGB1) and Carcinoembryonic Antigen Related Cell Adhesion Molecule 1 (CEACAM1). The term “TIM-3” as used herein includes human TIM3 (hTIM-3), variants, isoforms, and species homologs of hTIM-3, and analogs having at least one common epitope. The term “GAL9” as used herein includes human GAL9 (hGAL9), variants, isoforms, and species homologs of hGAL9, and analogs having at least one common epitope. The term “PdtSer” as used herein includes variants and analogs having at least one common epitope. The term “HMGB1” as used herein includes human HMGB1 (hHMGB1), variants, isoforms, and species homologs of hHMGB1, and analogs having at least one common epitope. The term “CEACAM1” as used herein includes human CEACAM1 (hCEACAM1), variants, isoforms, and species homologs of hCEACAM1, and analogs having at least one common epitope. “CD94/NKG2A” is an inhibitory receptor predominantly expressed on the surface of natural killer cells and of CD8⁺ T cells. The term “CD94/NKG2A” as used herein includes human CD94/NKG2A (hCD94/NKG2A), variants, isoforms, and species homologs of hCD94/NKG2A, and analogs having at least one common epitope. The CD94/NKG2A receptor is a heterodimer comprising CD94 and NKG2A. It suppresses NK cell activation and CD8⁺ T cell function, probably by binding to ligands such as HLA-E. CD94/NKG2A restricts cytokine release and cytotoxic response of natural killer cells (NK cells), Natural Killer T cells (NK-T cells) and T cells (a/B and y/8). NKG2A is frequently expressed in tumor infiltrating cells and HLA-E is overexpressed in several cancers.

“Indoleamine 2,3-dioxygenase” (IDO) is a tryptophan catabolic enzyme with immune-inhibitory properties. The term “IDO” as used herein includes human IDO (hIDO), variants, isoforms, and species homologs of hIDO, and analogs having at least one common epitope. IDO is the rate limiting enzyme in tryptophan degradation catalyzing its conversion to kynurenine. Therefore, IDO is involved in depletion of essential amino acids. It is known to be involved in suppression of T and NK cells, generation and activation of T_{regs} and myeloid-derived suppressor cells, and promotion of tumor angiogenesis. IDO is overexpressed in many cancers and was shown to promote immune system escape of tumor cells and to facilitate chronic tumor progression when induced by local inflammation.

In the “adenosinergic pathway” or “adenosine signaling pathway” as used herein ATP is converted to adenosine by the ectonucleotidases CD39 and CD73 resulting in inhibitory signaling through adenosine binding by one or more of the inhibitory adenosine receptors “Adenosine A2A Receptor” (A2AR, also known as ADORA2A) and “Adenosine A2B Receptor” (A2BR, also known as ADORA2B). Adenosine is a nucleoside with immunosuppressive properties and is present in high concentrations in the tumor microenvironment restricting immune cell infiltration, cytotoxicity and cytokine production. Thus, adenosine signaling is a strategy of cancer cells to avoid host immune system clearance. Adenosine signaling through A2AR and A2BR is an important checkpoint in cancer therapy that is activated by high adenosine concentrations typically present in the tumor microenvironment. CD39, CD73, A2AR and A2BR are expressed by most immune cells, including T cells, invariant natural killer cells, B cells, platelets, mast cells and eosinophils. Adenosine signaling through A2AR and A2BR counteracts T cell receptor mediated activation of immune cells and results in increased numbers of T_{regs} and decreased activation of DCs and effector T cells. The term “CD39” as used herein includes human CD39 (hCD39), variants, isoforms, and species homologs of hCD39, and analogs having at least one common epitope. The term “CD73” as used herein includes human CD73 (hCD73), variants, isoforms, and species homologs of hCD73, and analogs having at least one common epitope. The term “A2AR” as used herein includes human A2AR (hA2AR), variants, isoforms, and species homologs of hA2AR, and analogs having at least one common epitope. The term “A2BR” as used herein includes human A2BR (hA2BR), variants, isoforms, and species homologs of hA2BR, and analogs having at least one common epitope.

“V-domain Ig suppressor of T cell activation” (VISTA, also known as C10orf54) bears homology to PD-L1 but displays a unique expression pattern restricted to the hematopoietic compartment. The term “VISTA” as used herein includes human VISTA (hVISTA), variants, isoforms, and species homologs of hVISTA, and analogs having at least one common epitope. VISTA induces T cell suppression and is expressed by leukocytes within tumors.

The “Sialic acid binding immunoglobulin type lectin” (Siglec) family members recognize sialic acids and are involved in distinction between “self” and “non-self”. The term “Siglecs” as used herein includes human Siglecs (hSiglecs), variants, isoforms, and species homologs of hSiglecs, and analogs having at least one common epitope with one or more hSiglecs. The human genome contains 14 Siglecs of which several are involved in immunosuppression, including, without limitation, Siglec-2, Siglec-3, Siglec-7 and Siglec-9. Siglec receptors bind glycans containing sialic acid, but differ in their recognition of the linkage regiochemistry and spatial distribution of sialic residues. The members of the family also have distinct expression patterns. A broad range of malignancies overexpress one or more Siglecs.

“CD20” is an antigen expressed on the surface of B and T cells. High expression of CD20 can be found in cancers, such as B cell lymphomas, hairy cell leukemia, B cell chronic lymphocytic leukemia, and melanoma cancer stem cells. The term “CD20” as used herein includes human CD20 (hCD20), variants, isoforms, and species homologs of hCD20, and analogs having at least one common epitope.

“Glycoprotein A repetitions predominant” (GARP) plays a role in immune tolerance and the ability of tumors to escape the patient's immune system. The term “GARP” as used herein includes human GARP (hGARP), variants, isoforms, and species homologs of hGARP, and analogs having at least one common epitope. GARP is expressed on lymphocytes including Treg cells in peripheral blood and tumor infiltrating T cells at tumor sites. It probably binds to latent “transforming growth factor B” (TGF-β). Disruption of GARP signaling in T_{regs} results in decreased tolerance and inhibits migration of T_{regs} to the gut and increased proliferation of cytotoxic T cells.

“CD47” is a transmembrane protein that binds to the ligand “signal-regulatory protein alpha” (SIRPa). The term “CD47” as used herein includes human CD47 (hCD47), variants, isoforms, and species homologs of hCD47, and analogs having at least one common epitope with hCD47. The term “SIRPa” as used herein includes human SIRPa (hSIRPa), variants, isoforms, and species homologs of hSIRPa, and analogs having at least one common epitope with hSIRPa. CD47 signaling is involved in a range of cellular processes including apoptosis, proliferation, adhesion and migration. CD47 is overexpressed in many cancers and functions as “don't eat me” signal to macrophages. Blocking CD47 signaling through inhibitory anti-CD47 or anti-SIRPa antibodies enables macrophage phagocytosis of cancer cells and fosters the activation of cancer-specific T lymphocytes.

“Poliovirus receptor related immunoglobulin domain containing” (PVRIG, also known as CD112R) binds to “Poliovirus receptor-related 2” (PVRL2). PVRIG and PVRL2 are overexpressed in a number of cancers. PVRIG expression also induces TIGIT and PD-1 expression and PVRL2 and PVR (a TIGIT ligand) are co-overexpressed in several cancers. Blockade of the PVRIG signaling pathway results in increased T cell function and CD8⁺ T cell responses and, therefore, reduced immune suppression and elevated interferon responses. The term “PVRIG” as used herein includes human PVRIG (hPVRIG), variants, isoforms, and species homologs of hPVRIG, and analogs having at least one common epitope with hPVRIG. “PVRL2” as used herein includes hPVRL2, as defined above.

The “colony-stimulating factor 1” pathway is another checkpoint that can be targeted according to the disclosure. CSF1R is a myeloid growth factor receptor that binds CSF1. Blockade of the CSF1R signaling can functionally reprogram macrophage responses, thereby enhancing antigen presentation and anti-tumor T cell responses. The term “CSF1R” as used herein includes human CSF1R (hCSF1R), variants, isoforms, and species homologs of hCSF1R, and analogs having at least one common epitope with hCSF1R. The term “CSF1” as used herein includes human CSF1 (hCSF1), variants, isoforms, and species homologs of hCSF1, and analogs having at least one common epitope with hCSF1.

“Nicotinamide adenine dinucleotide phosphate NADPH oxidase” refers to an enzyme of the NOX family of enzymes of myeloid cells that generate immunosuppressive reactive oxygen species (ROS). Five NOX enzymes (NOX1 to NOX5) have been found to be involved in cancer development and immunosuppression. Elevated ROS levels have been detected in almost all cancers and promote many aspects of tumor development and progression. NOX produced ROS dampens NK and T cell functions and inhibition of NOX in myeloid cells improves anti-tumor functions of adjacent NK cells and T cells. The term “NOX” as used herein includes human NOX (hNOX), variants, isoforms, and species homologs of hNOX, and analogs having at least one common epitope with hNOX.

Another immune checkpoint that can be targeted according to the disclosure is the signal mediated by “tryptophan-2,3-dioxygenase” (TDO). TDO represents an alternative route to IDO in tryptophan degradation and is involved in immune suppression. Since tumor cells may catabolize tryptophan via TDO instead of IDO, TDO may represent an additional target for checkpoint blockade. Indeed, several cancer cell lines have been found to upregulate TDO and TDO may complement IDO inhibition. The term “TDO” as used herein includes human TDO (hTDO), variants, isoforms, and species homologs of hTDO, and analogs having at least one common epitope with hTDO.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins, which is modulated according to the present disclosure is typically the regulation of T cell activation, T cell proliferation and/or T cell function. Immune checkpoint proteins thus regulate and maintain self-tolerance and the duration and amplitude of physiological immune responses. Many of the immune checkpoint proteins belong to the B7:CD28 family or to the tumor necrosis factor receptor (TNFR) super family and, by binding to specific ligands, activate signaling molecules that are recruited to the cytoplasmic domain (Suzuki et al., 2016, Jap J Clin Onc, 46:191-203).

As used herein, the term “immune checkpoint modulator” or “checkpoint modulator” refers to a molecule or to a compound that modulates the function of one or more checkpoint proteins. Immune checkpoint modulators are typically able to modulate self-tolerance and/or the amplitude and/or the duration of the immune response. Preferably, the immune checkpoint modulator used according to the present disclosure modulates the function of one or more human checkpoint proteins and is, thus, a “human checkpoint modulator”. In a preferred embodiment, the human checkpoint modulator as used herein is an immune checkpoint inhibitor.

As used herein, “immune checkpoint inhibitor” or “checkpoint inhibitor” refers to a molecule that totally or partially reduces, inhibits, interferes with or negatively modulates one or more checkpoint proteins or that totally or partially reduces, inhibits, interferes with or negatively modulates expression of one or more checkpoint proteins. In certain embodiments, the immune checkpoint inhibitor binds to one or more checkpoint proteins. In certain embodiments, the immune checkpoint inhibitor binds to one or more molecules regulating checkpoint proteins. In certain embodiments, the immune checkpoint inhibitor binds to precursors of one or more checkpoint proteins e.g., on DNA- or RNA-level. Any agent that functions as a checkpoint inhibitor according to the present disclosure can be used.

The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In certain embodiments, the immune checkpoint inhibitor suitable for use in the methods disclosed herein, is an antagonist of inhibitory signals, e.g., an antibody which targets, for example, PD-1, PD-L1, CTLA-4, LAG-3, B7-H3, B7-H4, or TIM-3. These ligands and receptors are reviewed in Pardoll, D., Nature. 12: 252-264, 2012. Further immune checkpoint proteins that can be targeted according the disclosure are described herein.

In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule inhibitor that disrupts inhibitory signaling. In certain embodiments, the immune checkpoint inhibitor is a peptide-based inhibitor that disrupts inhibitory signaling. In certain embodiments, the immune checkpoint inhibitor is an inhibitory nucleic acid molecule that disrupts inhibitory signaling.

In certain embodiments, the immune checkpoint inhibitor is an antibody, fragment thereof, or antibody mimic, that prevents the interaction between checkpoint blocker proteins, e.g., an antibody, or fragment thereof that prevents the interaction between PD-1 and PD-L1 or PD-L2. In certain embodiments, the immune checkpoint inhibitor is an antibody, fragment thereof, or antibody mimic, that prevents the interaction between CTLA-4 and CD80 or CD86. In certain embodiments, the immune checkpoint inhibitor is an antibody, fragment thereof, or antibody mimic, that prevents the interaction between LAG-3 and its ligands, or TIM-3 and its ligands. In certain embodiments, the immune checkpoint inhibitor prevents inhibitory signaling through CD39 and/or CD73 and/or the interaction of A2AR and/or A2BR with adenosine. In certain embodiments, the immune checkpoint inhibitor prevents interaction of B7-H3 with its receptor and/or of B7-H4 with its receptor. In certain embodiments, the immune checkpoint inhibitor prevents the interaction of BTLA with its ligand HVEM. In certain embodiments, the immune checkpoint inhibitor prevents the interaction of one or more KIRs with their respective ligands. In certain embodiments, the immune checkpoint inhibitor prevents the interaction of LAG-3 with one or more of its ligands. In certain embodiments, the immune checkpoint inhibitor prevents the interaction of TIM-3 with one or more of its ligands Galectin-9, PtdSer, HMGB1 and CEACAM1. In certain embodiments, the immune checkpoint inhibitor prevents the interaction of TIGIT with one or more of its ligands PVR, PVRL2 and PVRL3. In certain embodiments, the immune checkpoint inhibitor prevents the interaction of CD94/NKG2A with HLA-E. In certain embodiments, the immune checkpoint inhibitor prevents the interaction of VISTA with one or more of its binding partners. In certain embodiments, the immune checkpoint inhibitor prevents the interaction of one or more Siglecs and their respective ligands. In certain embodiments, the immune checkpoint inhibitor prevents CD20 signaling. In certain embodiments, the immune checkpoint inhibitor prevents the interaction of GARP with one or more of its ligands. In certain embodiments, the immune checkpoint inhibitor prevents the interaction of CD47 with SIRPa. In certain embodiments, the immune checkpoint inhibitor prevents the interaction of PVRIG with PVRL2. In certain embodiments, the immune checkpoint inhibitor prevents the interaction of CSF1R with CSF1. In certain embodiments, the immune checkpoint inhibitor prevents NOX signaling. In certain embodiments, the immune checkpoint inhibitor prevents IDO and/or TDO signaling.

Inhibiting or blocking of inhibitory immune checkpoint signaling, as described herein, results in preventing or reversing immune-suppression and establishment or enhancement of T cell immunity against cancer cells. In one embodiment, inhibition of immune checkpoint signaling, as described herein, reduces or inhibits dysfunction of the immune system. In one embodiment, inhibition of immune checkpoint signaling, as described herein, renders dysfunctional immune cells less dysfunctional. In one embodiment, inhibition of immune checkpoint signaling, as described herein, renders a dysfunctional T cell less dysfunctional.

The term “dysfunction”, as used herein, refers to a state of reduced immune responsiveness to antigenic stimulation. The term includes the common elements of both exhaustion and/or anergy in which antigen recognition may occur, but the ensuing immune response is ineffective to control infection or tumor growth. Dysfunction also includes a state in which antigen recognition is retarded due to dysfunctional immune cells.

The term “dysfunctional”, as used herein, refers to an immune cell that is in a state of reduced immune responsiveness to antigen stimulation. Dysfunctional includes unresponsive to antigen recognition and impaired capacity to translate antigen recognition into downstream T cell effector functions, such as proliferation, cytokine production (e.g., IL-2) and/or target cell killing.

The term “anergy”, as used herein, refers to the state of unresponsiveness to antigen stimulation resulting from incomplete or insufficient signals delivered through the T cell receptor (TCR). T cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T cells do not undergo clonal expansion and/or acquire effector functions.

The term “exhaustion”, as used herein, refers to immune cell exhaustion, such as T cell exhaustion as a state of T cell dysfunction that arises from sustained TCR signaling that occurs during many chronic infections and cancer. It is distinguished from anergy in that it arises not through incomplete or deficient signaling, but from sustained signaling. Exhaustion is defined by poor effector function, sustained expression of inhibitory receptors and a transcriptional state distinct from that of functional effector or memory T cells. Exhaustion prevents optimal control of diseases (e.g., infection and tumors). Exhaustion can result from both extrinsic negative regulatory pathways (e.g., immunoregulatory cytokines) as well as cell intrinsic negative regulatory pathways (inhibitory immune checkpoint pathways, such as described herein).

“Enhancing T cell function” means to induce, cause or stimulate a T cell to have a sustained or amplified biological function, or renew or reactivate exhausted or inactive T cells. Examples of enhancing T cell function include increased secretion of y-interferon from CD8⁺ T cells, increased proliferation, increased antigen responsiveness (e.g., tumor clearance) relative to such levels before the intervention. In one embodiment, the level of enhancement is as least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 200%, or more. Manners of measuring this enhancement are known to one of ordinary skill in the art.

The immune checkpoint inhibitor may be an inhibitory nucleic acid molecule. The term “inhibitory nucleic acid” or “inhibitory nucleic acid molecule” as used herein refers to a nucleic acid molecule, e.g., DNA or RNA, that totally or partially reduces, inhibits, interferes with or negatively modulates one or more checkpoint proteins. Inhibitory nucleic acid molecules include, without limitation, oligonucleotides, siRNA, shRNA, antisense DNA or RNA molecules, and aptamers (e.g., DNA or RNA aptamers).

The term “oligonucleotide” as used herein refers to a nucleic acid molecule that is able to decrease protein expression, in particular expression of a checkpoint protein, such as the checkpoint proteins described herein. Oligonucleotides are short DNA or RNA molecules, typically comprising from 2 to 50 nucleotides. Oligonucleotides maybe single-stranded or double-stranded. A checkpoint inhibitor oligonucleotide may be an antisense-oligonucleotide. Antisense-oligonucleotides are single-stranded DNA or RNA molecules that are complementary to a given sequence, in particular to a sequence of the nucleic acid sequence (or a fragment thereof) of a checkpoint protein. Antisense RNA is typically used to prevent protein translation of mRNA, e.g., of mRNA encoding a checkpoint protein, by binding to said mRNA. Antisense DNA is typically used to target a specific, complementary (coding or non-coding) RNA. If binding takes place, such a DNA/RNA hybrid can be degraded by the enzyme RNase h. Moreover, morpholino antisense oligonucleotides can be used for gene knockdowns in vertebrates. For example, Kryczek et al., 2006 (J Exp Med, 203:871-81) designed B7-H4-specific morpholinos that specifically blocked B7-H4 expression in macrophages, resulting in increased T cell proliferation and reduced tumor volumes in mice with tumor associated antigen (TAA)-specific T cells.

The terms “siRNA” or “small interfering RNA” or “small inhibitory RNA” are used interchangeably herein and refer to a double-stranded RNA molecule with a typical length of 20-25 base pairs that interferes with expression of a specific gene, such as a gene coding for a checkpoint protein, with a complementary nucleotide sequence. In one embodiment, siRNA interferes with mRNA therefore blocking translation, e.g., translation of an immune checkpoint protein. Transfection of exogenous siRNA may be used for gene knockdown, however, the effect maybe only transient, especially in rapidly dividing cells. Stable transfection may be achieved, e.g., by RNA modification or by using an expression vector. Useful modifications and vectors for stable transfection of cells with siRNA are known in the art. siRNA sequences may also be modified to introduce a short loop between the two strands resulting in a “small hairpin RNA” or “shRNA”. shRNA can be processed into a functional siRNA by Dicer. shRNA has a relatively low rate of degradation and turnover. Accordingly, the immune checkpoint inhibitor may be a shRNA.

The term “aptamer” as used herein refers to a single-stranded nucleic acid molecule, such as DNA or RNA, typically in a length of 25-70 nucleotides that is capable of binding to a target molecule, such as a polypeptide. In one embodiment, the aptamer binds to an immune checkpoint protein such as the immune checkpoint proteins described herein. For example, an aptamer according to the disclosure can specifically bind to an immune checkpoint protein or polypeptide, or to a molecule in a signaling pathway that modulates the expression of an immune checkpoint protein or polypeptide. The generation and therapeutic use of aptamers is well known in the art (see, e.g., U.S. Pat. No. 5,475,096).

The terms “small molecule inhibitor” or “small molecule” are used interchangeably herein and refer to a low molecular weight organic compound, usually up to 1000 daltons, that totally or partially reduces, inhibits, interferes with, or negatively modulates one or more checkpoint proteins as described above. Such small molecular inhibitors are usually synthesized by organic chemistry, but may also be isolated from natural sources, such as plants, fungi, and microbes. The small molecular weight allows a small molecule inhibitor to rapidly diffuse across cell membranes. For example, various A2AR antagonists known in the art are organic compounds having a molecular weight below 500 daltons.

The immune checkpoint inhibitor may be an antibody, an antigen-binding fragment thereof, an antibody mimic or a fusion protein comprising an antibody portion with an antigen-binding fragment of the required specificity. Antibodies or antigen-binding fragments thereof are as described herein. Antibodies or antigen-binding fragments thereof that are immune checkpoint inhibitors include in particular antibodies or antigen-binding fragments thereof that bind to immune checkpoint proteins, such as immune checkpoint receptors or immune checkpoint receptor ligands. Antibodies or antigen-binding fragments may also be conjugated to further moieties, as described herein. In particular, antibodies or antigen-binding fragments thereof are chimerized, humanized or human antibodies. Preferably, immune checkpoint inhibitor antibodies or antigen-binding fragments thereof are antagonists of immune checkpoint receptors or of immune checkpoint receptor ligands.

In a preferred embodiment, an antibody that is an immune checkpoint inhibitor, is an isolated antibody.

The antibody that is an immune checkpoint inhibitor or the antigen-binding fragment thereof according to the present disclosure may also be an antibody that cross-competes for antigen binding with any known immune checkpoint inhibitor antibody. In certain embodiments, an immune checkpoint inhibitor antibody cross-competes with one or more of the immune checkpoint inhibitor antibodies described herein. The ability of antibodies to cross-compete for binding to an antigen indicates that these antibodies may bind to the same epitope region of the antigen or when binding to another epitope sterically hinder the binding of known immune checkpoint inhibitor antibodies to that particular epitope region. These cross-competing antibodies may have functional properties very similar to those they are cross-competing with as they are expected to block binding of the immune checkpoint to its ligand either by binding to the same epitope or by sterically hindering the binding of the ligand. Cross-competing antibodies can be readily identified based on their ability to cross-compete with one or more of known antibodies in standard binding assays such as Surface Plasmon Resoncance analysis, ELISA assays or flow cytometry (see, e.g., WO 2013/173223).

In certain embodiments, antibodies or antigen binding fragments thereof that cross-compete for binding to a given antigen with, or bind to the same epitope region of a given antigen as, one or more known antibodies are monoclonal antibodies. For administration to human patients, these cross-competing antibodies can be chimeric antibodies, or humanized or human antibodies. Such chimeric, humanized or human monoclonal antibodies can be prepared and isolated by methods well known in the art.

The checkpoint inhibitor may also be in the form of the soluble form of the molecules (or variants thereof) themselves, e.g., a soluble PD-L1 or PD-L1 fusion.

In the context of the disclosure, more than one checkpoint inhibitor can be used, wherein the more than one checkpoint inhibitors are targeting distinct checkpoint pathways or the same checkpoint pathway. Preferably, the more than one checkpoint inhibitors are distinct checkpoint inhibitors. Preferably, if more than one distinct checkpoint inhibitor is used, in particular at least 2, 3, 4, 5, 6, 7, 8, 9 or 10 distinct checkpoint inhibitors are used, preferably 2, 3, 4 or 5 distinct checkpoint inhibitors are used, more preferably 2, 3 or 4 distinct checkpoint inhibitors are used, even more preferably 2 or 3 distinct checkpoint inhibitors are used and most preferably 2 distinct checkpoint inhibitors are used. Preferred examples of combinations of distinct checkpoint inhibitors include combination of an inhibitor of PD-1 signaling and an inhibitor of CTLA-4 signaling, an inhibitor of PD-1 signaling and an inhibitor of TIGIT signaling, an inhibitor of PD-1 signaling and an inhibitor of B7-H3 and/or B7-H4 signaling, an inhibitor of PD-1 signaling and an inhibitor of BTLA signaling, an inhibitor of PD-1 signaling and an inhibitor of KIR signaling, an inhibitor of PD-1 signaling and an inhibitor of LAG-3 signaling, an inhibitor of PD-1 signaling and an inhibitor of TIM-3 signaling, an inhibitor of PD-1 signaling and an inhibitor of CD94/NKG2A signaling, an inhibitor of PD-1 signaling and an inhibitor of IDO signaling, an inhibitor of PD-1 signaling and an inhibitor of adenosine signaling, an inhibitor of PD-1 signaling and an inhibitor of VISTA signaling, an inhibitor of PD-1 signaling and an inhibitor of Siglec signaling, an inhibitor of PD-1 signaling and an inhibitor of CD20 signaling, an inhibitor of PD-1 signaling and an inhibitor of GARP signaling, an inhibitor of PD-1 signaling and an inhibitor of CD47 signaling, an inhibitor of PD-1 signaling and an inhibitor of PVRIG signaling, an inhibitor of PD-1 signaling and an inhibitor of CSF1R signaling, an inhibitor of PD-1 signaling and an inhibitor of NOX signaling, and an inhibitor of PD-1 signaling and an inhibitor of TDO signaling. In certain embodiments, the inhibitory immunoregulator (immune checkpoint blocker) is a component of the PD-1/PD-L1 or PD-1/PD-L2 signaling pathway. Accordingly, certain embodiments of the disclosure provide for administering to a subject a checkpoint inhibitor of the PD-1 signaling pathway. In certain embodiments, the checkpoint inhibitor of the PD-1 signaling pathway is a PD-1 inhibitor. In certain embodiments, the checkpoint inhibitor of the PD-1 signaling pathway is a PD-1 ligand inhibitor, such as a PD-L1 inhibitor or a PD-L2 inhibitor. In a preferred embodiment, the checkpoint inhibitor of the PD-1 signaling pathway is an antibody or an antigen-binding portion thereof that disrupts the interaction between the PD-1 receptor and one or more of its ligands, PD-L1 and/or PD-L2. Antibodies which bind to PD-1 and disrupt the interaction between PD-1 and one or more of its ligands are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity. In certain embodiments, the antibody or antigen-binding portion thereof binds specifically to PD-L2 and inhibits its interaction with PD-1, thereby increasing immune activity.

In certain embodiments, the inhibitory immunoregulator is a component of the CTLA-4 signaling pathway. Accordingly, certain embodiments of the disclosure provide for administering to a subject a checkpoint inhibitor of the CTLA-4 signaling pathway. In certain embodiments, the checkpoint inhibitor of the CTLA-4 signaling pathway is a CTLA-4 inhibitor. In certain embodiments, the checkpoint inhibitor of the CTLA-4 signaling pathway is a CTLA-4 ligand inhibitor.

In certain embodiments, the inhibitory immunoregulator is a component of the TIGIT signaling pathway. Accordingly, certain embodiments of the disclosure provide for administering to a subject a checkpoint inhibitor of the TIGIT signaling pathway. In certain embodiments, the checkpoint inhibitor of the TIGIT signaling pathway is a TIGIT inhibitor. In certain embodiments, the checkpoint inhibitor of the TIGIT signaling pathway is a TIGIT ligand inhibitor.

In certain embodiments, the inhibitory immunoregulator is a component of the B7 family signaling pathway. In certain embodiments, the B7 family members are B7-H3 and B7-H4. Certain embodiments of the disclosure provide for administering to a subject a checkpoint inhibitor of B7-H3 and/or B7-4. Accordingly, certain embodiments of the disclosure provide for administering to a subject an antibody or an antigen-binding portion thereof that targets B7-H3 or B7-H4. The B7 family does not have any defined receptors but these ligands are upregulated on tumor cells or tumor-infiltrating cells. Preclinical mouse models have shown that blockade of these ligands can enhance anti-tumor immunity.

In certain embodiments, the inhibitory immunoregulator is a component of the BTLA signaling pathway. Accordingly, certain embodiments of the disclosure provide for administering to a subject a checkpoint inhibitor of the BTLA signaling pathway. In certain embodiments, the checkpoint inhibitor of the BTLA signaling pathway is a BTLA inhibitor. In certain embodiments, the checkpoint inhibitor of the BTLA signaling pathway is a HVEM inhibitor.

In certain embodiments, the inhibitory immunoregulator is a component of one or more KIR signaling pathways. Accordingly, certain embodiments of the disclosure provide for administering to a subject a checkpoint inhibitor of one or more KIR signaling pathways. In certain embodiments, the checkpoint inhibitor of one or more KIR signaling pathways is a KIR inhibitor. In certain embodiments, the checkpoint inhibitor one or more KIR signaling pathways is a KIR ligand inhibitor. For example, the KIR inhibitor according to the present disclosure may be an anti-KIR antibody that binds to KIR2DL1, KIR2DL2, and/or KIR2DL3.

In certain embodiments, the inhibitory immunoregulator is a component of the LAG-3 signaling pathway. Accordingly, certain embodiments of the disclosure provide for administering to a subject a checkpoint inhibitor of LAG-3 signaling. In certain embodiments, the checkpoint inhibitor of the LAG-3 signaling pathway is a LAG-3 inhibitor. In certain embodiments, the checkpoint inhibitor of the LAG-3 signaling pathway is a LAG-3 ligand inhibitor.

In certain embodiments, the inhibitory immunoregulator is a component of the TIM-3 signaling pathway. Accordingly, certain embodiments of the disclosure provide for administering to a subject a checkpoint inhibitor of the TIM-3 signaling pathway. In certain embodiments, the checkpoint inhibitor of the TIM-3 signaling pathway is a TIM-3 inhibitor. In certain embodiments, the checkpoint inhibitor of the TIM-3 signaling pathway is a TIM-3 ligand inhibitor.

In certain embodiments, the inhibitory immunoregulator is a component of the CD94/NKG2A signaling pathway. Accordingly, certain embodiments of the disclosure provide for administering to a subject a checkpoint inhibitor of the CD94/NKG2A signaling pathway. In certain embodiments, the checkpoint inhibitor of the CD94/NKG2A signaling pathway is a CD94/NKG2A inhibitor. In certain embodiments, the checkpoint inhibitor of the CD94/NKG2A signaling pathway is a CD94/NKG2A ligand inhibitor. In certain embodiments, the inhibitory immunoregulator is a component of the IDO signaling pathway. Accordingly, certain embodiments of the disclosure provide for administering to a subject a checkpoint inhibitor of the IDO signaling pathway, e.g., an IDO inhibitor.

In certain embodiments, the inhibitory immunoregulator is a component of the adenosine signaling pathway. Accordingly, certain embodiments of the disclosure provide for administering to a subject a checkpoint inhibitor of the adenosine signaling pathway. In certain embodiments, the checkpoint inhibitor of the adenosine signaling pathway is a CD39 inhibitor. In certain embodiments, the checkpoint inhibitor of the adenosine signaling pathway is a CD73 inhibitor. In certain embodiments, the checkpoint inhibitor of the adenosine signaling pathway is an A2AR inhibitor. In certain embodiments, the checkpoint inhibitor of the adenosine signaling pathway is an A2BR inhibitor.

In certain embodiments, the inhibitory immunoregulator is a component of the VISTA signaling pathway. Accordingly, certain embodiments of the disclosure provide for administering to a subject a checkpoint inhibitor of the VISTA signaling pathway. In certain embodiments, the checkpoint inhibitor of the VISTA signaling pathway is a VISTA inhibitor.

In certain embodiments, the inhibitory immunoregulator is a component of one or more Siglec signaling pathways. Accordingly, certain embodiments of the disclosure provide for administering to a subject a checkpoint inhibitor of one or more Siglec signaling pathways. In certain embodiments, the checkpoint inhibitor of one or more Siglec signaling pathways is a Siglec inhibitor. In certain embodiments, the checkpoint inhibitor of one or more Siglec signaling pathways is a Siglec ligand inhibitor.

In certain embodiments, the inhibitory immunoregulator is a component of the CD20 signaling pathway. Accordingly, certain embodiments of the disclosure provide for administering to a subject a checkpoint inhibitor of the CD20 signaling pathway. In certain embodiments, the checkpoint inhibitor of the CD20 signaling pathway is a CD20 inhibitor.

In certain embodiments, the inhibitory immunoregulator is a component of the GARP signaling pathway. Accordingly, certain embodiments of the disclosure provide for administering to a subject a checkpoint inhibitor of the GARP signaling pathway. In certain embodiments, the checkpoint inhibitor of the GARP signaling pathway is a GARP inhibitor.

In certain embodiments, the inhibitory immunoregulator is a component of the CD47 signaling pathway. Accordingly, certain embodiments of the disclosure provide for administering to a subject a checkpoint inhibitor of the CD47 signaling pathway. In certain embodiments, the checkpoint inhibitor of the CD47 signaling pathway is a CD47 inhibitor. In certain embodiments, the checkpoint inhibitor of the CD47 signaling pathway is a SIRPa inhibitor.

In certain embodiments, the inhibitory immunoregulator is a component of the PVRIG signaling pathway. Accordingly, certain embodiments of the disclosure provide for administering to a subject a checkpoint inhibitor of the PVRIG signaling pathway. In certain embodiments, the checkpoint inhibitor of the PVRIG signaling pathway is a PVRIG inhibitor. In certain embodiments, the checkpoint inhibitor of the PVRIG signaling pathway is a PVRIG ligand inhibitor.

In certain embodiments, the inhibitory immunoregulator is a component of the CSF1R signaling pathway. Accordingly, certain embodiments of the disclosure provide for administering to a subject a checkpoint inhibitor of the CSF1R signaling pathway. In certain embodiments, the checkpoint inhibitor of the CSF1R signaling pathway is a CSF1R inhibitor. In certain embodiments, the checkpoint inhibitor of the CSF1R signaling pathway is a CSF1 inhibitor.

In certain embodiments, the inhibitory immunoregulator is a component of the NOX signaling pathway. Accordingly, certain embodiments of the disclosure provide for administering to a subject a checkpoint inhibitor of the NOX signaling pathway, e.g., a NOX inhibitor.

In certain embodiments, the inhibitory immunoregulator is a component of the TDO signaling pathway. Accordingly, certain embodiments of the disclosure provide for administering to a subject a checkpoint inhibitor of the TDO signaling pathway, e.g., a TDO inhibitor.

Exemplary PD-1 inhibitors include, without limitation, anti-PD-1 antibodies such as BGB-A317 (BeiGene; see U.S. Pat. No. 8,735,553, WO 2015/35606 and US 2015/0079109), cemiplimab (Regeneron; see WO 2015/112800) and lambrolizumab (e.g., disclosed as hPD109A and its humanized derivatives h409A1, h409A16 and h409A17 in WO2008/156712), AB137132 (Abcam), EH12.2H7 and RMP1-14 (#BE0146; Bioxcell Lifesciences Pvt. LTD.), MIH4 (Affymetrix eBioscience), nivolumab (OPDIVO, BMS-936558; Bristol Myers Squibb; see WO 2006/121168), pembrolizumab (KEYTRUDA; MK-3475; Merck; see WO 2008/156712), pidilizumab (CT-011; CureTech; see Hardy et al., 1994, Cancer Res., 54(22):5793-6 and WO 2009/101611), PDR001 (Novartis; see WO 2015/112900), MEDI0680 (AMP-514; AstraZeneca; see WO 2012/145493), TSR-042 (see WO 2014/179664), REGN-2810 (H4H7798N; cf. US 2015/0203579), JS001 (TAIZHOU JUNSHI PHARMA; see Si-Yang Liu et al., 2007, J. Hematol. Oncol. 70: 136), AMP-224 (GSK-2661380; cf. Li et al., 2016, Int J Mol Sci 17(7):1151 and WO 2010/027827 and WO 2011/066342), PF-06801591 (Pfizer), BGB-A317 (BeiGene; see WO 2015/35606 and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANBO11; see WO2014/179664), GLS-010 (Wuxi/Harbin Gloria Pharmaceuticals; also known as WBP3055; see Si-Yang et al., 2017, J. Hematol. Oncol. 70: 136), STI-1110 (Sorrento Therapeutics; see WO 2014/194302), AGEN2034 (Agenus; see WO 2017/040790), mgA012 (Macrogenics; see WO 2017/19846), IBI308 (Innovent; see WO 2017/024465, WO 2017/025016, WO 2017/132825, and WO 2017/133540), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105,

U.S. Pat. Nos. 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28(9):665-678 and Sasikumar and Ramachandra, 2018, BioDrugs, 32(5):481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831.

In a certain embodiment, the PD-1 inhibitor is nivolumab (OPDIVO; BMS-936558), pembrolizumab (KEYTRUDA; MK-3475), pidilizumab (CT-011), PDR001, MEDI0680 (AMP-514), TSR-042, REGN2810, JS001, AMP-224 (GSK-2661380), PF-06801591, BGB-A317, BI 754091, or SHR-1210.

Exemplary PD-1 ligand inhibitors are PD-L1 inhibitors and PD-L2 inhibitors and include, without limitation, anti-PD-L1 antibodies such as MEDI4736 (durvalumab; AstraZeneca; see WO 2011/066389), MSB-0010718C (see US 2014/0341917), YW243.55.S70 (see SEQ ID NO: 20 of WO 2010/077634 and U.S. Pat. No. 8,217,149), MIH1 (Affymetrix eBioscience; cf. EP 3 230 319), MDX-1105 (Roche/Genentech; see WO2013019906 and U.S. Pat. No. 8,217,149) STI-1014 (Sorrento; see WO2013/181634), CK-301 (Checkpoint Therapeutics), KN035 (3D Med/Alphamab; see Zhang et al., 2017, Cell Discov. 3:17004), atezolizumab (TECENTRIQ; RG7446; MPDL3280A; R05541267; see U.S. Pat. No. 9,724,413), BMS-936559 (Bristol Myers Squibb; see U.S. Pat. No. 7,943,743, WO 2013/173223), avelumab (bavencio; cf. US 2014/0341917), LY3300054 (Eli Lilly Co.), CX-072 (Proclaim-CX-072; also called CytomX; see WO2016/149201), FAZ053, KN035 (see WO2017020801 and WO2017020802), MDX-1105 (see US 2015/0320859), anti-PD-L1 antibodies disclosed in U.S. Pat. No. 7,943,743, including 3G10, 12A4 (also referred to as BMS-936559), 10A5, 5F8, 10H10, 1B12, 7H1, 11E6, 12B7, and 13G4, anti-PD-L1 antibodies as described in WO 2010/077634, U.S. Pat. No. 8,217,149, WO 2010/036959, WO 2010/077634, WO 2011/066342, U.S. Pat. Nos. 8,217,149, 7,943,743, WO 2010/089411, U.S. Pat. Nos. 7,635,757, 8,217,149, US 2009/0317368, WO 2011/066389, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO 2018/222711, WO2015/112805, WO2015/061668, WO2014/159562, WO2014/165082, WO2014/100079.

Exemplary CTLA-4 inhibitors include, without limitation, the monoclonal antibodies ipilimumab (Yervoy; Bristol Myers Squibb) and tremelimumab (Pfizer/MedImmune), trevilizumab, AGEN-1884 (Agenus) and ATOR-1015, the anti-CTLA4 antibodies disclosed in WO 2001/014424, US 2005/0201994, EP 1212422, U.S. Pat. Nos. 5,811,097, 5,855,887, 6,051,227, 6,682,736, 6,984,720, WO 01/14424, WO 00/37504, US 2002/0039581, US 2002/086014, WO 98/42752, U.S. Pat. Nos. 6,207,156, 5,977,318, 7,109,003, and 7,132,281, the dominant negative proteins abatacept (Orencia; see EP 2 855 533), which comprises the Fe region of IgG 1 fused to the CTLA-4 ECD, and belatacept (Nulojix; see WO 2014/207748), a second generation higher-affinity CTLA-4-Ig variant with two amino acid substitutions in the CTLA-4 ECD relative to abatacept, soluble CTLA-4 polypeptides, e.g., RG2077 and CTLA4-IgG4m (see U.S. Pat. No. 6,750,334), anti-CTLA-4 aptamers and siRNAs directed to CTLA-4, e.g., as disclosed in US 2015/203848. Exemplary CTLA-4 ligand inhibitors are described in Pile et al., 2015 (Encyclopedia of Inflammatory Diseases, M. Parnham (ed.), doi: 10.1007/978-3-0348-0620-6_20).

Exemplary checkpoint inhibitors of the TIGIT signaling pathway include, without limitation, anti-TIGIT antibodies, such as BMS-986207, COM902 (CGEN-15137; Compugen), AB154 (Arcus Biosciences) or etigilimab (OMP-313M32; OncoMed Pharmaceuticals), or the antibodies disclosed in WO2017/059095, in particular “MAB10”, US 2018/0185482, WO 2015/009856, and US 2019/0077864.

Exemplary checkpoint inhibitors of B7-H3 include, without limitation, the Fc-optimized monoclonal antibody enoblituzumab (MGA271; Macrogenics; see US 2012/0294796) and the anti-B7-H3 antibodies mgD009 (Macrogenics) and pidilizumab (see U.S. Pat. No. 7,332,582).

Exemplary B7-H4 inhibitors include, without limitation, antibodies as described in Dangaj et al., 2013 (Cancer Research 73:4820-9) and in Smith et al., 2014 (Gynecol Oncol, 134:181-189), WO 2013/025779 (e.g., 2D1 encoded by SEQ ID NOs: 3 and 4, 2H9 encoded by SEQ ID NO: 37 and 39, and 2E11 encoded by SEQ ID NOs: 41 and 43) and in WO 2013/067492 (e.g., an antibody with an amino acid sequence selected from SEQ ID NOs: 1-8), morpholino antisense oligonucleotides, e.g., as described by Kryczek et al., 2006 (J Exp Med, 203:871-81), or soluble recombinant forms of B7-H4, such as disclosed in US 2012/0177645. Exemplary BTLA inhibitors include, without limitation, the anti-BTLA antibodies described in Crawford and Wherry, 2009 (J Leukocyte Biol 86:5-8), WO 2011/014438 (e.g., 4C7 or an antibody comprising heavy and light chains according to SEQ ID NOs: 8 and 15 and/or SEQ ID NOs: 11 and 18), WO 2014/183885 (e.g., the antibody deposited under the number CNCM I-4752) and US 2018/155428.

Checkpoint inhibitors of KIR signaling include, without limitation, the monoclonal antibodies lirilumab (1-7F9; IPH2102; see U.S. Pat. No. 8,709,411), IPH4102 (Innate Pharma; see Marie-Cardine et al., 2014, Cancer 74(21): 6060-70), anti-KIR antibodies as disclosed, e.g., in US 2018/208652, US 2018/117147, US 2015/344576, WO 2005/003168, WO 2005/009465, WO 2006/072625, WO 2006/072626, WO 2007/042573, WO 2008/084106 (e.g., an antibody comprising heavy and light chains according to SEQ ID NOs: 2 and 3), WO 2010/065939, WO 2012/071411, WO 2012/160448 and WO 2014/055648.

LAG-3 inhibitors include, without limitation, the anti-LAG-3 antibodies BMS-986016 (Bristol-Myers Squibb; see WO 2014/008218 and WO 2015/116539), 25F7 (see US2011/0150892), IMP731 (see WO 2008/132601), H5L7BW (cf. WO2014140180), MK-4280 (28G-10; Merck; see WO 2016/028672), REGN3767 (Regneron/Sanofi), BAP050 (see WO 2017/019894), IMP-701 (LAG-525; Novartis) Sym022 (Symphogen), TSR-033 (Tesaro), mgD013 (a bispecific DART antibody targeting LAG-3 and PD-1 developed by MacroGenics), BI754111 (Boehringer Ingelheim), FS118 (a bispecific antibody targeting LAG-3 and PD-1 developed by F-star), GSK2831781 (GSK) and antibodies as disclosed in WO 2009/044273, WO 2008/132601, WO 2015/042246, EP 2 320 940, US 2019/169294, US 2019/169292, WO 2016/028672, WO 2016/126858, WO 2016/200782, WO 2015/200119, WO 2017/220569, WO 2017/087589, WO 2017/219995, WO 2017/019846, WO 2017/106129, WO 2017/062888, WO 2018/071500, WO 2017/087901, US 2017/0260271, WO 2017/198741, WO2017/220555, WO2017/015560, WO2017/025498, WO2017/149143, WO 2018/069500, WO2018/083087, WO2018/034227 WO2014/140180, the LAG-3 antagonistic protein AVA-017 (Avacta), the soluble LAG-3 fusion protein IMP321 (eftilagimod alpha; Immutep; see EP 2 205 257 and Brignone et al., 2007, J. Immunol., 179: 4202-4211), and soluble LAG-3 proteins disclosed in WO 2018/222711.

TIM-3 inhibitors include, without limitation, antibodies targeting TIM-3 such as F38-2E2 (BioLegend), cobolimab (TSR-022; Tesaro), LY3321367 (Eli Lilly), MBG453 (Novartis) and antibodies as disclosed in, e.g., WO 2013/006490, WO 2018/085469 (e.g., antibodies comprising heavy and light chain sequences encoded by nucleic acid sequences according to SEQ ID NOs: 3 and 4), WO 2018/106588, WO 2018/106529 (e.g., an antibody comprising heavy and light chain sequences according to SEQ ID NOs: 8-11).

TIM-3 ligand inhibitors include, without limitation, CEACAM1 inhibitors such as the anti-CEACAM1 antibody CM10 (cCAM Biotherapeutics; see WO 2013/054331), antibodies disclosed in WO 2015/075725 (e.g., CM-24, 26H7, 5F4, TEC-11, 12-140-4, Apr. 3, 2017, COL-4, F36-54, 34B1, YG-C28F2, D14HD11, M8.7.7, D11-AD11, HEA81, B I. I, CLB-gran-10, F34-187, T84.1, B6.2, B 1.13, YG-C94G7, 12-140-5, scFv DIATHIS1, TET-2; cCAM Biotherapeutics), antibodies described by Watt et al., 2001 (Blood, 98: 1469-1479) and in WO 2010/12557 and PtdSer inhibitors such as bavituximab (Peregrine).

CD94/NKG2A inhibitors include, without limitation, monalizumab (IPH2201; Innate Pharma) and the antibodies and method for their production as disclosed in U.S. Pat. No. 9,422,368 (e.g., humanized Z199; see EP 2 628 753), EP 3 193 929 and WO2016/032334 (e.g., humanized Z270; see EP 2 628 753).

IDO inhibitors include, without limitation, exiguamine A, epacadostat (INCB024360; InCyte; see U.S. Pat. No. 9,624,185), indoximod (Newlink Genetics; CAS #: 110117-83-4), NLG919 (Newlink Genetics/Genentech; CAS #: 1402836-58-1), GDC-0919 (Newlink Genetics/Genentech; CAS #: 1402836-58-1), F001287 (Flexus Biosciences/BMS; CAS #: 2221034-29-1), KHK2455 (Cheong et al., 2018, Expert Opin Ther Pat. 28(4):317-330), PF-06840003 (see WO 2016/181348), navoximod (RG6078, GDC-0919, NLG919; CAS #: 1402837-78-8), linrodostat (BMS-986205; Bristol-Myers Suibb; CAS #: 1923833-60-6), small molecules such as 1-methyl-tryptophan, pyrrolidine-2,5-dione derivatives (see WO 2015/173764) and the IDO inhibitors disclosed by Sheridan, 2015, Nat Biotechnol 33:321-322.

CD39 inhibitors include, without limitation, A001485 (Arcus Biosciences), PSB 069 (CAS #: 78510-31-3) and the anti-CD39 monoclonal antibody IPH5201 (Innate Pharma; see Perrot et al., 2019, Cell Reports 8:2411-2425.E9).

CD73 inhibitors include, without limitation, anti-CD73 antibodies such as CPI-006 (Corvus Pharmaceuticals), MEDI9447 (MedImmune; see WO2016075099), IPH5301 (Innate Pharma; see Perrot et al., 2019, Cell Reports 8:2411-2425.E9), the anti-CD73 antibodies described in WO2018/110555, the small molecule inhibitors PBS 12379 (Tocris Bioscience; CAS #: 1802226-78-3), A000830, A001190 and A001421 (Arcus Biosciences; see Becker et al., 2018, Cancer Research 78(13 Supplement):3691-3691, doi: 10.1158/1538-7445.AM2018-3691), CB-708 (Calithera Biosciences) and purine cytotoxic nucleoside analogue-based diphosphonates as described by Allard et al., 2018 (Immunol Rev., 276(1): 121-144).

A2AR inhibitors include, without limitation, small molecule inhibitors such as istradefylline (KW-6002; CAS #: 155270-99-8), PBF-509 (Palobiopharma), ciforadenant (CPI-444: Corvus Pharma/Genentech; CAS #: 1202402-40-1), ST1535 ([2butyl-9-methyl-8-(2H-1,2,3-triazol 2-yl)-9H-purin-6-xylamine]; CAS #: 496955-42-1), ST4206 (see Stasi et al., 2015, Europ J Pharm 761:353-361; CAS #: 1246018-36-9), tozadenant (SYN115; CAS #: 870070-55-6), V81444 (see WO 2002/055082), preladenant (SCH420814; Merck; CAS #: 377727-87-2), vipadenant (BIIB014; CAS #: 442908-10-3), ST1535 (CAS #: 496955-42-1), SCH412348 (CAS #: 377727-26-9), SCH442416 (Axon 2283; Axon Medchem; CAS #: 316173-57-6), ZM241385 (4-(2-(7-amino-2-(2-furyl)-(1,2,4)triazolo(2,3-a)-(1,3,5)triazin-5-yl-amino)ethyl)phenol; Cas #: 139180-30-6), AZD4635 (AstraZeneca), AB928 (a dual A2AR/A2BR small molecule inhibitor; Arcus Biosciences) and SCH58261 (see Popoli et al., 2000, Neuropsychopharm 22:522-529; CAS #: 160098-96-4).

A2BR inhibitors include, without limitation, AB928 (a dual A2AR/A2BR small molecule inhibitor; Arcus Biosciences), MRS 1706 (CAS #: 264622-53-9), GS6201 (CAS #: 752222-83-6) and PBS 1115 (CAS #: 152529-79-8).

VISTA inhibitors include, without limitation, anti-VISTA antibodies such as JNJ-61610588 (onvatilimab; Janssen Biotech) and the small molecule inhibitor CA-170 (anti-PD-L1/L2 and anti-VISTA small molecule; CAS #: 1673534-76-3).

Siglec inhibitors include, without limitation, the anti-Sigle-7 antibodies disclosed in US 2019/023786 and WO 2018/027203 (e.g., an antibody comprising a variable heavy chain region according to SEQ ID NO: 1 and a variable light chain region according to SEQ ID NO: 15), the anti-Siglec-2 antibody inotuzumab ozogamicin (Besponsa; see U.S. Pat. Nos. 8,153,768 and 9,642,918), the anti-Siglec-3 antibody gemtuzumab ozogamicin (Mylotarg; see U.S. Pat. No. 9,359,442) or the anti-Siglec-9 antibodies disclosed in US 2019/062427, US 2019/023786, WO 2019/011855, WO 2019/011852 (e.g., an antibody comprising the CDRs according to SEQ ID NOs: 171-176, or 3 and 4, or 5 and 6, or 7 and 8, or 9 and 10, or 11 and 12, or 13 and 14, or 15 and 16, or 17 and 18, or 19 and 20, or 21 and 22, or 23 and 24, or 25 and 26), US 2017/306014 and EP 3 146 979.

CD20 inhibitors include, without limitation, anti-CD20 antibodies such as rituximab (RITUXAN; IDEC-102; IDEC-C2B8; see U.S. Pat. No. 5,843,439), ABP 798 (rituximab biosimilar), ofatumumab (2F2; see WO2004/035607), obinutuzumab, ocrelizumab (2h7; see WO 2004/056312), ibritumomab tiuxetan (Zevalin), tositumomab, ublituximab (LFB-R603; LFB Biotechnologies) and the antibodies disclosed in US 2018/0036306 (e.g., an antibody comprising light and heavy chains according to SEQ ID NOs: 1-3 and 4-6, or 7 and 8, or 9 and 10).

GARP inhibitors include, without limitation, anti-GARP antibodies such as ARGX-115 (arGEN-X) and the antibodies and methods for their production as disclosed in US 2019/127483, US 2019/016811, US 2018/327511, US 2016/251438, EP 3 253 796.

CD47 inhibitors include, without limitation, anti-CD47 antibodies such as HuF9-G4 (Stanford University/Forty Seven), CC-90002/INBRX-103 (Celgene/Inhibrx), SRF231 (Surface Oncology), IBI188 (Innovent Biologics), AO-176 (Arch Oncology), bispecific antibodies targeting CD47 including TG-1801 (NI-1701; bispecific monoclonal antibody targeting CD47 and CD19; Novimmune/TG Therapeutics) and NI-1801 (bispecific monoclonal antibody targeting CD47 and mesothelin; Novimmune), and CD47 fusion proteins such as ALX 148 (ALX Oncology; see Kauder et al., 2019, PLOS One, doi: 10.1371/journal.pone.0201832).

SIRPa inhibitors include, without limitation, anti-SIRPa antibodies such as OSE-172 (Boehringer Ingelheim/OSE), FSI-189 (Forty Seven), anti-SIRPa fusion proteins such as TTI-621 and TTI-662 (Trillium Therapeutics; see WO 2014/094122).

PVRIG inhibitors include, without limitation, anti-PVRIG antibodies such as COM701 (CGEN-15029) and antibodies and method for their manufacture as disclosed in, e.g., WO 2018/033798 (e.g., CHA.7.518.1H4(S241P), CHA.7.538.1.2.H4(S241P), CPA.9.086H4(S241P), CPA.9.083H4(S241P), CHA.9.547.7.H4(S241P), CHA.9.547.13.H4(S241P) and antibodies comprising a variable heavy domain according to SEQ ID NO: 5 and a variable light domain according to SEQ ID NO: 10 of WO 2018/033798 or antibodies comprising a heavy chain according to SEQ ID NO:9 and a light chain according to SEQ ID NO: 14; WO 2018/033798 further discloses anti-TIGIT antibodies and combination therapies with anti-TIGIT and anti-PVRIG antibodies), WO2016134333, WO2018017864 (e.g., an antibody comprising a heavy chain according to SEQ ID NOs: 5-7 having at least 90% sequence identity to SEQ ID NO: 11 and/or a light chain according to SEQ ID NOs: 8-10 having at least 90% sequence identity to SEQ ID NO: 12, or an antibody encoded by SEQ ID NOs: 13 and/or 14 or SEQ ID NOs: 24 and/or 29, or another antibody disclosed in WO 2018/017864) and anti-PVRIG antibodies and fusion peptides as disclosed in WO 2016/134335.

CSF1R inhibitors include, without limitation, anti-CSF1R antibodies cabiralizumab (FPA008; FivePrime; see WO 2011/140249, WO 2013/169264 and WO 2014/036357), IMC-CS4 (EliLilly), emactuzumab (R05509554; Roche), RG7155 (WO 2011/70024, WO 2011/107553, WO 2011/131407, WO 2013/87699, WO 2013/119716, WO 2013/132044) and the small molecule inhibitors BLZ945 (CAS #: 953769-46-5) and pexidartinib (PLX3397; Selleckchem; CAS #: 1029044-16-3).

CSF1 inhibitors include, without limitation, anti-CSF1 antibodies disclosed in EP 1 223 980 and Weir et al., 1996 (J Bone Mineral Res 11: 1474-1481), WO 2014/132072, and antisense DNA and RNA as disclosed in WO 2001/030381.

Exemplary NOX inhibitors include, without limitation, NOX1 inhibitors such as the small molecule ML171 (Gianni et al., 2010, ACS Chem Biol 5(10):981-93, NOS31 (Yamamoto et al., 2018, Biol Pharm Bull. 41(3):419-426), NOX2 inhibitors such as the small molecules ceplene (histamine dihydrochloride; CAS #: 56-92-8), BJ-1301 (Gautam et al., 2017, Mol Cancer Ther 16(10):2144-2156; CAS #: 1287234-48-3) and inhibitors described by Lu et al., 2017, Biochem Pharmacol 143:25-38, NOX4 inhibitors such as the small molecule inhibitors VAS2870 (Altenhofer et al., 2012, Cell Mol Life Sciences 69(14):2327-2343), diphenylene iodonium (CAS #: 244-54-2) and GKT137831 (CAS #: 1218942-37-0; see Tang et al., 2018, 19(10):578-585).

TDO inhibitors include, without limitation, 4-(indol-3-yl)-pyrazole derivatives (see U.S. Pat. No. 9,126,984 and US 2016/0263087), 3-indol substituted derivatives (see WO 2015/140717, WO 2017/025868, WO 2016/147144), 3-(indol-3-yl)-pyridine derivatives (see US 2015/0225367 and WO 2015/121812), dual IDO/TDO antagonist, such as small molecule dual IDO/TDO inhibitors disclosed in WO 2015/150097, WO 2015/082499, WO 2016/026772, WO 2016/071283, WO 2016/071293, WO 2017/007700, and the small molecule inhibitor CB548 (Kim, C, et al., 2018, Annals Oncol 29 (suppl_8): viii400-viii441).

According to the disclosure, the immune checkpoint inhibitor is an inhibitor of an inhibitory checkpoint protein but preferably not an inhibitor of a stimulatory checkpoint protein. As described herein, a number of CTLA-4, PD-1, TIGIT, B7-H3, B7-H4, BTLA, KIR, LAG-3, TIM-3, CD94/NKG2A, IDO, A2AR, A2BR, VISTA, Siglec, CD20, CD39, CD73, GARP, CD47, PVRIG, CSF1R, NOX and TDO inhibitors and inhibitors of respective ligands are known and several of them are already in clinical trials or even approved. Based on these known immune checkpoint inhibitors, alternative immune checkpoint inhibitors may be developed. In particular, known inhibitors of the preferred immune checkpoint proteins may be used as such or analogues thereof may be used, in particular chimerized, humanized or human forms of antibodies and antibodies cross-competing with any of the antibodies described herein.

It will be understood by one of ordinary skill in the art that other immune checkpoint targets can also be targeted by antagonists or antibodies, provided that the targeting results in the stimulation of an immune response such as an anti-tumor immune response as reflected in an increase in T cell proliferation, enhanced T cell activation, and/or increased cytokine production (e.g., IFN-γ, IL2).

Checkpoint inhibitors may be administered in any manner and by any route known in the art. The mode and route of administration will depend on the type of checkpoint inhibitor to be used.

Checkpoint inhibitors may be administered in the form of any suitable pharmaceutical composition as described herein.

Checkpoint inhibitors may be administered in the form of nucleic acid, such DNA or RNA molecules, encoding an immune checkpoint inhibitor, e.g., an inhibitory nucleic acid molecule or an antibody or fragment thereof. For example, antibodies can be delivered encoded in expression vectors, as described herein. Nucleic acid molecules can be delivered as such, e.g., in the form of a plasmid or mRNA molecule, or complexed with a delivery vehicle, e.g., a liposome, lipoplex or nucleic-acid lipid particles. Checkpoint inhibitors may also be administered via an oncolytic virus comprising an expression cassette encoding the checkpoint inhibitor. Checkpoint inhibitors may also be administered by administration of endogeneic or allogeneic cells able to express a checkpoint inhibitor, e.g., in the form of a cell based therapy. The term “cell based therapy” refers to the transplantation of cells (e.g., T lymphocytes, dendritic cells, or stem cells) expressing an immune checkpoint inhibitor into a subject for the purpose of treating a disease or disorder (e.g., a cancer disease). In one embodiment, the cell based therapy comprises genetically engineered cells. In one embodiment, the genetically engineered cells express an immune checkpoint inhibitor, such as described herein. In one embodiment, the genetically engineered cells express an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. Genetically engineered cells may also express further agents that enhance T cell function. Such agents are known in the art. Cell based therapies for the use in inhibition of immune checkpoint signaling are disclosed, e.g., in WO 2018/222711, herein incorporated by reference in its entirety.

The term “oncolytic virus” as used herein, refers to a virus capable of selectively replicating in and slowing the growth or inducing the death of a cancerous or hyperproliferative cell, either in vitro or in vivo, while having no or minimal effect on normal cells. An oncolytic virus for the delivery of an immune checkpoint inhibitor comprises an expression cassette that may encode an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. The oncolytic virus preferably is replication competent and the expression cassette is under the control of a viral promoter, e.g., synthetic early/late poxvirus promoter. Exemplary oncolytic viruses include vesicular stomatitis virus (VSV), rhabdoviruses (e.g., picornaviruses such as Seneca Valley virus; SVV-001), coxsackievirus, parvovirus, Newcastle disease virus (NDV), herpes simplex virus (HSV; OncoVEX GMCSF), retroviruses (e.g., influenza viruses), measles virus, reovirus, Sinbis virus, vaccinia virus, as exemplarily described in WO 2017/209053 (including Copenhagen, Western Reserve, Wyeth strains), and adenovirus (e.g., Delta-24, Delta-24-RGD, ICOVIR-5, ICOVIR-7, Onyx-015, ColoAd1, H101, AD5/3-D24-GMCSF). Generation of recombinant oncolytic viruses comprising a soluble form of an immune checkpoint inhibitor and methods for their use are disclosed in WO 2018/022831, herein incorporated by reference in its entirety. Oncolytic viruses can be used as attenuated viruses.

### Pharmaceutical Compositions

The agents described herein may be administered in pharmaceutical compositions or medicaments and may be administered in the form of any suitable pharmaceutical composition.

A pharmaceutical composition may comprise a pharmaceutically acceptable carrier and may optionally comprise one or more adjuvants, stabilizers etc. In one embodiment, a pharmaceutical composition is for therapeutic or prophylactic treatments, e.g., for use in treating or preventing cancer.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, IL10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In one embodiment, the RNA encoding an IL7 immunostimulant, in particular IL7 fused to human serum albumin, is administed at a dose of between 30 μg/kg RNA to 180 μg/kg. In one embodiment, the RNA encoding an IL2 immunostimulant, in particular IL2 fused to human serum albumin, is administed at a dose of between 0.4 μg/kg RNA to 120 μg/kg.

In some embodiments, an effective amount comprises an amount sufficient to cause a tumor/lesion to shrink. In some embodiments, an effective amount is an amount sufficient to decrease the growth rate of a tumor (such as to suppress tumor growth). In some embodiments, an effective amount is an amount sufficient to delay tumor development. In some embodiments, an effective amount is an amount sufficient to prevent or delay tumor recurrence. In some embodiments, an effective amount is an amount sufficient to increase a subject's immune response to a tumor, such that tumor growth and/or size and/or metastasis is reduced, delayed, ameliorated, and/or prevented. An effective amount can be administered in one or more administrations. In some embodiments, administration of an effective amount (e.g., of a composition comprising mRNAs) may: (i) reduce the number of cancer cells; (ii) reduce tumor size; (iii) inhibit, retard, slow to some extent and may stop cancer cell infiltration into peripheral organs; (iv) inhibit (e.g., slow to some extent and/or block or prevent) metastasis; (v) inhibit tumor growth; (vi) prevent or delay occurrence and/or recurrence of tumor; and/or (vii) relieve to some extent one or more of the symptoms associated with the cancer.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients. Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for systemic administration, e.g., for intravenous administration.

The term “co-administering” as used herein means a process whereby different compounds or compositions (e.g., RNA encoding an antigen and RNA encoding an immunostimulant) are administered to the same patient. The different compounds or compositions may be administered simultaneously, at essentially the same time, or sequentially.

### Treatments

The present invention provides methods and agents for inducing an immune response, in particular for inducing an immune response against a target antigen or cells expressing a target antigen, e.g., tumor cells expressing a target antigen, in a subject comprising administering an effective amount of a composition comprising RNA encoding an immunostimulant and optionally RNA encoding a vaccine antigen described herein.

In one embodiment, the methods and agents described herein provide immunity in a subject to a disease or disorder associated with a target antigen. The present invention thus provides methods and agents for treating or preventing the disease, or disorder associated with the target antigen.

In one embodiment, the methods and agents described herein are administered to a subject having a disease, or disorder associated with a target antigen. In one embodiment, the methods and agents described herein are administered to a subject at risk for developing the disease, or disorder associated with the target antigen.

The therapeutic compounds or compositions of the invention may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In some embodiments, administration of a composition of the present invention may be performed by single administration or boosted by multiple administrations.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against cancer cells, and to treat a cancer disease. In one embodiment, the cancer is an antigen-positive cancer. In one embodiment, the cancer is advanced solid tumors such as metastatic (Stage IV) or unresectable localized cancer.

Pharmaceutical compositions described herein are applicable for inducing or enhancing an immune response. Pharmaceutical compositions described herein are thus useful in a prophylactic and/or therapeutic treatment of a disease involving an antigen or epitope.

As used herein, “immune response” refers to an integrated bodily response to an antigen or a cell expressing an antigen and refers to a cellular immune response and/or a humoral immune response. The immune system is divided into a more primitive innate immune system, and acquired or adaptive immune system of vertebrates, each of which contains humoral and cellular components.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to immune effector cells, in particular to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells.

The term “effector functions” in the context of the present invention includes any functions mediated by components of the immune system that result, for example, in the killing of diseased cells such as cancer cells. In one embodiment, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present invention relates to a cell which exerts effector functions during an immune reaction. An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells, most preferably CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system. Upon activation, cytotoxic lymphocytes trigger the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells.

A “lymphoid cell” is a cell which is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted and preferably exerts effector functions of T cells.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class Il molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

A majority of T cells have a T cell receptor (TCR) existing as a complex of several proteins. The TCR of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. The actual T cell receptor is composed of two separate peptide chains, which are produced from the independent T cell receptor alpha and beta (TCRa and TCRB) genes and are called a- and B-TCR chains. y8 T cells (gamma delta T cells) represent a small subset of T cells that possess a distinct T cell receptor (TCR) on their surface. However, in y8 T cells, the TCR is made up of one y-chain and one 8-chain. This group of T cells is much less common (2% of total T cells) than the aß T cells.

“Humoral immunity” or “humoral immune response” is the aspect of immunity that is mediated by macromolecules found in extracellular fluids such as secreted antibodies, complement proteins, and certain antimicrobial peptides. It contrasts with cell-mediated immunity. Its aspects involving antibodies are often called antibody-mediated immunity.

Humoral immunity refers to antibody production and the accessory processes that accompany it, including: Th2 activation and cytokine production, germinal center formation and isotype switching, affinity maturation and memory cell generation. It also refers to the effector functions of antibodies, which include pathogen neutralization, classical complement activation, and opsonin promotion of phagocytosis and pathogen elimination.

In humoral immune response, first the B cells mature in the bone marrow and gain B-cell receptors (BCR's) which are displayed in large number on the cell surface. These membrane-bound protein complexes have antibodies which are specific for antigen detection. Each B cell has a unique antibody that binds with an antigen. The mature B cells migrate from the bone marrow to the lymph nodes or other lymphatic organs, where they begin to encounter pathogens. When a B cell encounters an antigen, the antigen is bound to the receptor and taken inside the B cell by endocytosis. The antigen is processed and presented on the B cell's surface again by MHC-II proteins. The B cell waits for a helper T cell (T_{H}) to bind to the complex. This binding will activate the T_{H} cell, which then releases cytokines that induce B cells to divide rapidly, making thousands of identical clones of the B cell. These daughter cells either become plasma cells or memory cells. The memory B cells remain inactive here; later when these memory B cells encounter the same antigen due to reinfection, they divide and form plasma cells. On the other hand, the plasma cells produce a large number of antibodies which are released free into the circulatory system. These antibodies will encounter antigens and bind with them. This will either interfere with the chemical interaction between host and foreign cells, or they may form bridges between their antigenic sites hindering their proper functioning, or their presence will attract macrophages or killer cells to attack and phagocytose them.

The term “antibody” includes an immunoglobulin comprising at least two heavy (H) chains and two light (L) chains inter-connected by disulfide bonds. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An antibody binds, preferably specifically binds with an antigen.

Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

An “antibody heavy chain”, as used herein, refers to the larger of the two types of polypeptide chains present in antibody molecules in their naturally occurring conformations.

An “antibody light chain”, as used herein, refers to the smaller of the two types of polypeptide chains present in antibody molecules in their naturally occurring conformations, K and A light chains refer to the two major antibody light chain isotypes.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination. The present disclosure provides for the provision of immunostimulants to a subject for inducing an immune response. The immune response that is induced by providing immunostimulants may be an immune response that occurs without a vaccine being provided to a subject. In one embodiment, the immune response is an immune response that is induced by endogenous antigen. Alternatively, vaccine antigen may be additionally provided to a subject, preferably in the form of RNA encoding the vaccine antigen.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class Il molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class Il molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” refers to any disease which implicates an antigen, e.g. a disease which is characterized by the presence of an antigen. The disease involving an antigen can be an infectious disease or cancer. As mentioned above, the antigen may be a disease-associated antigen, such as a tumor antigen or viral antigen. In one embodiment, a disease involving an antigen is a disease involving cells expressing an antigen.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

One particular form of cancer that can be treated by the compositions and methods described herein is advanced solid tumors such as metastatic (Stage IV) or unresectable localized cancer. The term “cancer” according to the disclosure also comprises cancer metastases.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Test Compounds

### Introduction to BNT152 and BNT153

BNT152 and BNT153 are lipid nanoparticle (LNP) formulated ribonucleic acids (RNA) coding for human interleukin (IL)-7 fused to the N-terminus of human serum albumin (hAlb) and for human IL-2 fused to the C-terminus of hAlb (hIL7-hAlb and hAlb-hIL2, respectively) (FIG. 1). The drug product is an RNA-LNP for IV injection. The nanoparticle format protects IV administered RNA from extracellular RNases and was engineered for systemic delivery and targeting of the RNA to liver cells.

Each drug substance is a modified single-stranded, 5′-capped mRNA that is translated into hIL7-hAlb or hAlb-hIL2, respectively, upon entering liver cells. The general structure of the protein-encoding RNA, which is determined by the respective nucleotide sequence of the linearized plasmid DNA used as template for in vitro RNA transcription, is schematically illustrated in FIG. 2.

In addition to the sequence encoding the target protein (i.e., the open reading frame [ORF]), each RNA contains structural elements optimized for maximal efficacy of the RNA with respect to stability and translational efficiency (5′-cap, 5′-untranslated region [UTR], 3′-UTR, poly(A)-tail; FIG. 2). A so-called cap1 structure (m₂^{7,3′-O}Gppp(m₁^{2′-O})ApG) is a specific capping structure at the 5′-end of the RNA drug substances. The RNA drug substances are synthesized in the presence of N1-methylpseudouridine triphosphate (m¹WTP) instead of uridine triphosphate (UTP).

The nomenclature for the drug substances and the drug products is given in Table 1.

TABLE 1
Drug substance and drug product nomenclature
for BNT153 and BNT152

| | BNT153 |
|---|---|
| Chemical class: | RNA |
| Encoded protein: | Fusion protein of human albumin and human IL-2 |
| | (Uni ProtKB/Swiss-Prot Identifier = |
| | P02768 and Q309Q7, respectively) |
| Laboratory code drug | RBP006.1 |
| substance: | |
| Laboratory code drug | RBP006.1-DP |
| product: | |
| Chemical class: | RNA |
| Encoded protein: | Fusion protein of human IL-7 and human albumin |
| | (Uni ProtKB/Swiss-Prot Identifier = |
| | P13232 and P02768, respectively) |
| Laboratory code drug | RBP009.1 |
| substance: | |
| Laboratory code drug | RBP009.1-DP |
| product: | |

DP = drug product, DS = drug substance, hAlb = human albumin, IL = interleukin, RNA = ribonucleic acid.

BNT152 and BNT153 are members of the RiboCytokine® platform, a novel RNA-based technology designed to address the limitations of recombinantly expressed cytokines (FIG. 1).

The active pharmaceutical ingredient of RiboCytokine platform is single stranded, nucleoside-modified RNA engineered for minimal immunogenicity. RNA modification by incorporation of the nucleoside analog N1-methylpseudouridine reduces the recognition of transfected RNA by endosomal toll-like receptors (TLRs) and the subsequent TLR-mediated translational shutdown, hence leading to sustained protein production (Sahin U et al., Nat Rev Drug Discov 2014; 13(10): 759-80, Karikó K et al., Immunity 2005; 23(2): 165-75, Andries O et al., J Control Release 2015; 217: 337-44). To enable efficient translation and systemic availability of the translated protein, RNA is formulated with LNPs designed for delivery of the RNA to the liver as the main secretory organ after i.v. IV administration (Stadler C R et al., Nat Med 2017; 23(7): 815-17, FIG. 3A).

Biodistribution of RiboCytokine surrogates

To study the in vivo distribution and translation of RNA-LNPs, we treated BALB/c mice IV with 3 μg LNP-formulated RNA encoding firefly lucipherase (LUC) and monitored LUC expression for four days (FIG. 3A). LNPs were provided by Arbutus BioPharma as ready-to-use particles and were stored at −75° C. to −80° C. A vial of LUC RNA stock solution was resolved in nuclease-free water to obtain 0.5 μg/μL directly before use and was then diluted to 0.5 mg/mL LNP stock with DPBS. LNP-formulated LUC RNA were applied IV using 3/10cc insulin syringes with a 29G needle. Prior to IV injection, animals were anesthetized by inhalation of 2.5% isoflurane in oxygen.

Bioluminescence imaging of LUC expression was performed 6, 24, 48, 72, and 96 h after injection of LUC RNA using a Xenogen IVIS Spectrum in vivo Imaging System according to the manufacturer's instructions. Images were acquired five minutes after intraperitoneal (IP) injection of luciferase substrate D-luciferin at a dose of 150 mg/kg using an exposure time of 60 seconds to ensure that the signal acquired was within the effective detection range. Mice were anesthetized after receiving D-luciferin in a chamber with 2.5% isoflurane and placed on the imaging platform while being maintained on 2.5% isoflurane delivered via a nose cone. After acquisition, bioluminescence quantification was performed by Living Image software. The region of interest was manually marked around the signal area in the liver, and the emitted photons quantified by recording total flux (photons/seconds) and average radiance (photons/seconds/cm²/steradian).

Intravenous delivery of LNP-formulated LUC RNA resulted in selective luciferase activity in the liver for up to 96 h. No relevant bioluminescent signal was observed in any other region.

To improve the serum half-life of the encoded cytokines, cytokine sequences are fused to human serum albumin (hAlb). In addition to increasing the molecular size above the threshold for renal clearance, hAlb prevents lysosomal degradation of the fusion proteins, instead facilitating their salvage through binding of the membrane-bound neonatal Fc receptor, which leads to their release back into the circulation (Kontermann R E, Curr Opin Biotechnol 2011; 22(6): 868-76).

It was investigated how fusion to albumin changes the biodistribution, and particularly the availability of the encoded target in the circulation, tumor and tumor-draining lymph nodes (TDLNs) (FIG. 3B). To this end, we generated nucleoside-modified RNAs encoding a secreted variant of NanoLuc® luciferase (sec-nLUC) fused to murine albumin (sec-nLUC-mAlb).

The study consisted of one in vivo experiment with 18 female BALB/c mice. On Day 0 of the experiment, 18 mice were inoculated with CT26 murine colon carcinoma cells. On Day 24, the tumor-bearing mice were stratified into two treatment groups of eight each, and each mouse received a single treatment. Mice were treated IV with LNPs (TransIT, Mirus Bio) containing 3 μg of RNA encoding sec-nLUC or or sec-nLUC-mAlb. Two mice remained untreated and served as controls.

CT26 cells were cultured according to standard cell culture procedures. On Day 0 of the experiment, CT26 cells were harvested from a cell culture growing in log-phase (approx. 90% viability) and counted. The cell number was adjusted to 5×10⁶ cells/mL with PBS and cells were kept on ice until injection. Mice received a 100 μL subcutaneous (s.c.) injection into the upper flank corresponding to 5×10⁵ cells per mouse.

For the preparation of RNA-TransIT complexes, a total of 1,800 μl of material enough for nine animals was prepared for group 1 and group 2 each (200 μl per animal, plus enough for one extra animal). An RNase-free polypropylene tube was used for the mixing of the reagents. After addition of pre-chilled DMEM (4° C.) and TransIT reagents, the preparations were vortexed for 20 seconds, incubated for 2-5 minutes, and then immediately injected.

The RNA preparations were applied IV using a 29G needle. Prior to injection, animals were anesthetized by inhalation of 2.5% isoflurane in oxygen.

Blood was retrieved and serum prepared 2, 6, 24, 48, and 72 h after treatment from two to three animals per time-point per group. Liver, tumor, TDLNs and non-TDLNs (NDLNs) were isolated 6, 24, 48, and 72 h after treatment from two animals per time-point and group. Additionally, the two untreated, tumor-bearing control animals were euthanized five days after the last euthanasia time-point, and serum and tissues collected as above.

Organ collection was performed as follows. Following euthanasia, mice were disinfected with 70% ethanol and the dissection was performed starting with an abdominal incision. The spleen and draining lymph nodes were collected and stored in PBS on ice for subsequent single cell preparations.

Isolated TDLNs, NDLNs, tumor, and liver tissues were transferred into individual Precellys lysing tubes, leaving enough room for the lysing buffer. All tubes were snap-frozen in liquid nitrogen, kept on dry ice during transport, and stored at −80° C. For tissue lysate preparations, the cryopreserved tissues were thawed at ambient temperature. DPBS supplemented with protease and phosphatase inhibitors was added and tissues were homogenized using a tissue homogenizer. Lysates were cleared by centrifugation, and supernatants transferred into pre-chilled Eppendorf tubes and stored on ice. Protein concentrations were measured using a BCA protein assay kit according to the manufacturer's instructions. The lysates were snap-frozen in liquid nitrogen and stored at −80° C. until needed for the Nano-Glo luciferase assay.

The Nano-Glo luciferase assay was carried out according to the manufacturer's instructions using the lytic method. Briefly, 50 μl of Nano-Glo assay reagent was added to 50 μl of each sample lysate per 96-plate well, corresponding to 30 μg of tissue or 50 μl of serum. The plate containing the samples was incubated for 5 minutes at ambient temperature in the dark, then shaken for 5 seconds in a M200 Tecan plate reader followed by luminescence measurements. Luminescence measurements obtained from the tissues and serum of untreated animals served as background, which was subtracted from the corresponding tissue and serum test samples.

The luciferase assay results are plotted in FIG. 3B. Detectable expression of sec-nLUC was observed only in the liver, as this organ represents the primary transfection location of formulated mRNA as previously shown (Stadler C R et al., Nat Med 2017; 23(7): 815-17). Fusion of sec-nLUC with albumin, however, raised and prolonged systemic (serum) and intra-tumoral availability of luciferase. A mean of 5,857 relative light units (RLUs) in the tumor and 4,057,174 RLUs in the serum was observed in animals treated with sec-nLUC-mAlb, compared to-5 RLUs and 436 RLUs, respectively, in animals treated with sec-nLUC 72 h after injection. The fusion with albumin also led to distribution of the reporter protein to the TDLNs, with a mean of 1,611 RLUs compared to-5 RLUs in animals treated with sec-nLUC 24 h after injection.

Luciferase expression in the liver was highly similar 6 h after injection in both animal groups. However, 72 h after injection, a mean of 8,944 RLUs in animals treated with sec-nLUC-mAlb was observed, compared to 185 RLUs in animals treated with sec-nLUC. This indicates that albumin does not increase the expression of the translated protein but rather stabilizes it, thereby supporting prolonged availability. Overall, fusion of a secreted protein to albumin was shown to increase its bioavailability in tumors and tumor-draining lymph nodes (FIG. 3B).

In summary, the RiboCytokine platform technology addresses major limitations of recombinant cytokine therapies, i.e., short serum half-life, low bioavailability, and the resulting need for high and frequent dosing. We anticipate that a controlled release of cytokines via the RiboCytokine platform technology will improve safety as well as efficacy as compared to recombinant cytokines.

BNT152 and BNT153: Target background and rationale for their combination

The biological activity of IL-2 is mediated by binding to either a high-affinity heterotrimeric receptor that consists of IL-2Ra, IL-2RB and the common cytokine y chain (Yc), or a low-affinity heterodimeric receptor that comprises IL-2RB and yc (Liao W et al., Immunity 2013, 38(1): 13-25). Stimulation with IL-2 activates intracellular signaling through the Janus kinase/signal transducer and activator of transcription (Jak/STAT) and phosphatidylinositol-3 kinase (PI3K) pathways and supports the differentiation, proliferation, survival and effector functions of T cells (Gillis S, Smith K A, Nature 1977; 268(5616): 154-56, Blattman J N et al., Nat Med 2003; 9(5): 540-47, Bamford R N et al., Proc Natl Acad Sci USA. 1994; 91(11): 4940-44, Kamimura D, Bevan M J., J Exp Med 2007; 204(8): 1803-12). Activated tumor-specific CD4⁺ and CD8⁺ T cells are critical effector cells in cancer immunity and are the desired targets for activation by BNT153-translated hAlb-hIL2.

IL-7 signals through a heterodimeric receptor composed of IL-7Ra and Yc, which results in the activation of the Jak/STAT and PI3K pathways as well as Src family kinases (Fry T J, Mackall C L, Blood [Internet]. 2002 Jun. 1; 99(11): 3892-904, Available from =http://www.ncbi.nlm.nih.gov/pubmed/12010786). IL-7 plays an important role in T and B cell lymphopoiesis and survival as well as memory T cell formation (Fry T J, Mackall C L, Blood [Internet]. 2002 Jun. 1; 99(11): 3892-904, Available from =http://www.ncbi.nlm.nih.gov/pubmed/12010786, Cui G et al., Cell 2015; 161(4): 750-61). Injection of recombinant IL-7 was shown to expand CD8⁺ and CD4⁺ T cells while leading to a relative decrease of T_{regs} in humans (Rosenberg S A et al., J Immunother 2006; 29(3): 313-19). On the other hand, T_{regs}, known opponents of anti-tumor effector T cells, are elevated by IL-2 administration. Consequently, the anticipated mode of action of BNT152 is to amplify BNT153 mediated therapeutic efficacy via multiple mechanisms:

· Support of the generation of novel T cells (lymphopoiesis).

· Intensification of the tumor-specific T cell proliferation.

· Strengthening of the memory formation of anti-tumor T cells.

· Sensitization for BNT153 by upregulation of IL-2Ra on anti-tumor T cells. IL-2Ra together with the constitutively expressed IL-2RBy forms the high-affinity IL-2R.

· Reduction/normalization of the BNT153-mediated increase in the Treg proportion in CD4⁺ T cells.

The combined mode of action of BNT152 and BNT153 may form the basis for combination with T cell vaccines. Tumor antigen encoding RNA delivered via liposomal formulation (RNA lipoplex [RNA-LPX]) into antigen presenting cells mediates potent tumor-specific T cell responses (Kreiter S et al., Nature 2015; 520(7549): 692-96, Kranz L M et al., Nature 2016; 534(7607): 396-401). Expanded T cells were shown to express elevated levels of the high-affinity IL-2 receptor and are therefore particularly receptive for IL-2 therapy. Furthermore, as therapeutic activity of IL-2 and IL-7 depends on the support and expansion of pre-existing anti-tumor T cell responses, a T cell vaccination that generates tumor-specific T cells before or in parallel to treatment with BNT152 and BNT153 is expected to potentiate the effect of BNT152 and BNT153 treatment (Schwartzentruber D J et al., N Engl J Med 2011; 364(22): 2119-27).

BioNTech's RiboCytokines are expected to have a favorable safety profile and increased clinical efficacy compared to their recombinant counterparts. This notion is supported by the preclinical experiments, which are presented below.

BNT152 and BNT153 drug product description

The drug products BNT152 (RBP009.1-DP) and BNT153 (RBP006.1-DP) are preservative-free, sterile RNA-LNP dispersions in an aqueous cryoprotectant buffer for IV administration. The quantitative composition of the drug products is shown inTable 2.

TABLE 2
Quantitative composition of drug products

| | | | | Nominal |
|---|---|---|---|---|
| | Quality | | Concentration | amount per |
| Component | standard | Function | (mg/mL) | vial (mg/Vial) |
| RNA drug substance ¹ | In-house | Active pharmaceutical | 0.20 | 0.10 |
| | | ingredient | | |
| 3D-P-DMA ² | In-house | Functional lipid | 2.2 | 1.1 |
| PEG₂₀₀₀-C-DMA ³ | In-house | Functional lipid | 0.31 | 0.16 |
| DSPC ⁴ | In-house | Structural lipid | 0.60 | 0.30 |
| Cholesterol, synthetic | NF/Ph. Eur. | Structural lipid | 0.88 | 0.44 |
| Sucrose | NF/Ph. Eur. | Cryoprotectant | 100 | 50 |
| Maltose | NF | Cryoprotectant | 100 | 50 |
| Tris (2-Amino-2- | USP/Ph. Eur. | Buffer | 0.61 | 0.31 |
| hydroxymethyl- | | | | |
| propane-1,3-diol) | | | | |
| Hydrochloric acid | NF/Ph. Eur. | pH modifier | <0.11 | <0.055 |
| Water for injection | USP/Ph. Eur. | Solvent/Vehicle | q.s. | ad. 0.5 mL |

¹ RBP009.1 for BNT152 or RBP006.1 for BNT152.
² (6Z, 16Z)-12-((Z)-dec-4-en-1-yl)docosa-6,16-dien-11-yl 5-(dimethylamino)pentanoate.
³ 3-N-[(ω-Methoxy poly(ethylene glycol) 2000) carbamoyl]-1,2-dimyristyloxy-propylamine.
⁴ 1,2-Distearoyl-sn-glycero-3-phosphocholine.
DSPC = 1,2-dioctadecanoyl-sn-glycero-3-phosphocholineDSPCPC(18:0/18:0), NF = National Formulary, Ph. Eur. = European Pharmacopoeia (Pharmacopoeia Europaea), q.s. = quantum satis, RNA = ribonucleic acid.

The drug product contains four lipid excipients and a cryoprotectant buffer composed of 10% maltose, 10% sucrose, and 5 mM Tris buffer salt as shown in Table 2. The cryoprotectant buffer solution is adjusted to pH 8 using a hydrochloric acid solution.

The lipid excipients used in the drug product manufacturing process are the ionizable lipid 3D-P-DMA ((6Z,16Z)-12-((Z)-dec-4-en-1-yl)docosa-6,16-dien-11-yl 5-(dimethyl-amino)-pentanoate and the PEGylated lipid PEG₂₀₀₀-C-DMA (3-N-[(w-Methoxy poly(ethylene glycol)2000) carbamoyl]-1,2-dimyristyloxy-propylamine). Physicochemical properties and the structures of the 4 lipids are shown in Table 3.

TABLE 3
Lipid excipients in the drug products

| | Molecular | Molecular | Physical state and | |
|---|---|---|---|---|
| Lipid | weight g/mol | formula | storage condition | Chemical name (synonyms) and structure |
| 3D-P-DMA | 588 | C₃₉H₇₃NO₂ | Liquid (oil) | (6Z,16Z)-12-((Z)-dec-4-en-1-yl)docosa-6,16-dien-11-yl 5-(dimethylamino)pentanoate |
| | | | −20º C. | |
| | | | | 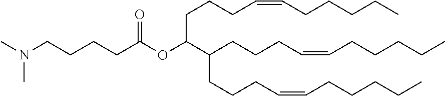 [SMILES: CCCCC/C=C\CCCC(CCC/C=C\CCCCC)C(CCC/C=C\CCCCC)OC(=O)CCCCN(C)C] |
| PEG₂₀₀₀-C- | 2700 ± 270 | CH₃O— | Solid | 3-N-[(ω-Methoxy poly(ethylene glycol)2000)carbamoyl]-1,2-dimyristyloxy- |
| DMA | n =~ 50 | (C₂H₄O)ₙ—CO— | −20º C. | propylamine |
| | | C₃₁H₆₄NO₂ | | (MPEG-(2 kDa)-C-DMA |
| | | | | Methoxy-polyethylene glycol-2,3-bis(tetradecyloxy)propylcarbamate (2000)) |
| | | | | 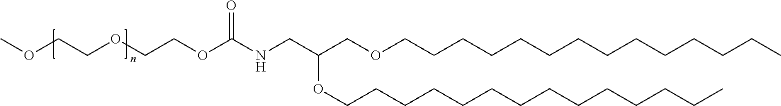 |
| DSPC¹ | 790 | C₄₄H₈₈NO₈P | Solid | 1,2-Distearoyl-sn-glycero-3-phosphocholine |
| | | | −20º C. | (1,2-dioctadecanoyl-sn-glycero-3-phosphocholine DSPCPC (18:0/18:0)) |
| | | | | 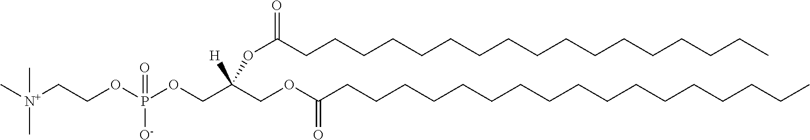 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol² | 387 | C₂₇H₄₆O | Solid | Cholest-5-en-3β-ol |
| | | | −20º C. | (3β-Hydroxy-5-cholestene5-Cholesten-3β-ol synthetic cholesterol SyntheChol) |
| | | | | 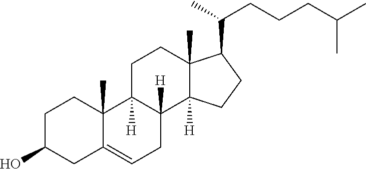 |

¹CAS Number 816-94-4.
²CAS number 57-88-5.

3D-P-DMA: The amino lipid, 3D-P-DMA, is the major lipid component of the drug product. 3D-P-DMA contains an ionizable tertiary amino head group linked via an ester bond to 3 mono-unsaturated alkyl chains which, when incorporated into the LNP, confer distinct physicochemical properties that regulate particle formation, cellular uptake, fusogenicity, and endosomal release of the RNA. 3D-P-DMA has an apparent pKa of approximately 6.3, so that at pH 5, the molecule is essentially fully positively charged. During the manufacturing process, introduction of an aqueous solution of RNA to an ethanolic lipid mixture containing 3D-P-DMA at pH 5 leads to an electrostatic interaction between the negatively charged RNA drug substance and the positively charged cationic lipid. This electrostatic interaction leads to particle formation coincident with efficient encapsulation of RNA drug substance. After RNA encapsulation, adjustment of the medium surrounding the resulting RNA-LNP to pH 8 results in neutralization of the surface charge on the LNP. When all other variables are held constant, charge-neutral particles display longer in vivo circulation lifetimes and better delivery to hepatocytes compared to charged particles, which are cleared rapidly by the reticuloendothelial system. Upon endosomal uptake, the low pH of the endosome renders the LNP fusogenic and allows for release of the RNA into the cytosol of the target cell. PEG₂₀₀₀-C-DMA: The PEGylated lipid PEG₂₀₀₀-C-DMA sterically stabilizes the particle by forming a protective hydrophilic layer that shields the hydrophobic lipid layer. By shielding the particles' surface, PEG₂₀₀₀-C-DMA prevents the association with serum proteins and the resulting uptake by the reticuloendothelial system when the particles are administered in vivo.

PEG₂₀₀₀-C-DMA has been selected for use in the drug product to provide optimum delivery of RNA to the liver. It has been found that by modulating the alkyl chain length of the PEG lipid anchor, the pharmacology of encapsulated nucleic acid can be controlled in a predictable manner. In the vial, the particles retain a full complement of PEG₂₀₀₀-C-DMA. In the blood compartment, PEG₂₀₀₀-C-DMA dissociates from the particle over time, revealing a more fusogenic particle that is more readily taken up by cells, ultimately leading to release of the RNA payload.

DSPC and cholesterol: The lipids DSPC and cholesterol can be referred to structural lipids with concentrations chosen to optimize LNP particle size, stability and encapsulation.

BNT152 and BNT153 RNA-LNP generation

RiboCytokine mRNA was generated by in vitro transcription based on Kreiter et al. (Kreiter, S. et al. Cancer Immunol. Immunother. 56, 1577-87 (2007)) with substitution of the nucleoside uridine by N1-methyl-pseudouridine. Resulting mRNAs were equipped with a Cap1-structure and double-stranded (dsRNA) molecules were depleted by cellulose purification (Baiersdorfer et al., Mol. Ther. (2019)). Purified mRNA was eluted in H₂O and stored at-60 to −80° C. until further use. In vitro transcription of all described mRNA constructs was carried out at BioNTech RNA Pharmaceuticals GmbH.

Unless specified otherwise, modified RNA was encapsulated within LNPs by Genevant Sciences Corporation (designated ‘Gen-LNPs’ in Example 9). These LNPs mediate preferential delivery of the RNA to the liver after IV administration. LNPs were stored at-60 to −80° C. For injection, LNP aliquots were thawed at ambient temperature and diluted to 100 μg/mL with PBS. Diluted LNPs were drawn into a 1 mL syringe with an 18G 11/2″ needle attached. The needle was replaced by a 13 mm 0.2 μm syringe filter and LNPs were slowly filtered into a fresh container. Filtered LNPs were further diluted to the final concentration with PBS.

The RNA preparations were applied IV using a 29G needle. Prior to IV injection, animals were anesthetized by inhalation of 2.5% isoflurane in oxygen.

### Example 2: Species Cross-Reactivity of hIL7-hAlb and hAlb-hIL2 to Mouse and Cynomolgus Monkey

To assess the activity of hIL7-hAlb and hAlb-hIL2 (translated proteins of the respective RiboCytokine RNAs) on human, cynomolgus monkey and mouse immune cells, freshly prepared PBMCs from these species were stimulated with the translated cytokines and assayed for STAT5 phosphorylation by flow cytometry. STAT5 protein is the common downstream mediator of the JAK-STAT pathway, being phosphorylated as one of the earliest signaling events mediated by IL-2 family cytokines, including IL-7 (Rani A, Murphy J J, J Interferon Cytokine Res 2016; 36(4): 226-37, Lin J X, Leonard W J, Oncogene 2000; 19(21): 2566-76). As such, phosphorylated STAT5 (pSTAT5) can be used as an objective and robust measure of cytokine bioactivity (Ehx G et al., Oncotarget 2015; 6(41): 43255-66, Kemp R A et al., Immunol Cell Biol 2010; 88(2):

213-19, Charych D et al., PLOS One 2017; 12(7): 1-24). The STAT5 amino acid sequence is conserved between species and ensures comparability of data obtained in all three species.

Species-specific bioactivity of each cytokine was determined on previously identified most responsive indicator immune cell subsets with CD4⁺CD25 T helper cells and CD8⁺ T cells as indicator populations for hIL7-hAlb activity, and CD4⁺CD25⁺ T_{regs} as indicator population for hAlb-hIL2 biological effect (FIG. 4). PBMCs of mouse, human, and cynomolgus macaque origin were collected by centrifugation (8 min, 300 xg, RT) and re-suspended in X-VIVO™ 15 serum-free hematopoietic cell medium. PBMCs were rested for 1 h at 37° C. and 5% CO₂. Next, 1.25×10⁵ cells were seeded per well of a 96-well V-bottom plate in 50 μL total volume and pre-warmed at 37° C. and 5% CO₂. In parallel, seven five-fold serial dilutions of hIL7-hAlb- and hAlb-hIL2-containing HEK293T/17 supernatants were prepared in X-VIVO™ 15. Seeded PBMCs were mixed 1:1 with diluted cytokine/serum albumin fusion construct supernatants and stimulated for 10 minutes (min) at 37° C. and 5% CO₂. Undiluted hAlb-containing supernatant was used as negative control. Fixable viability dye eFluor™ 780 was diluted 1:1,000 in DPBS and 10 μL of the dilution added per stimulated PBMC sample. After continuing the stimulation for another 5 min, the cells were fixed by addition of 100 μL Roti-Histofix 4% buffered formaldehyde solution and incubated for 10 min on ice (final formaldehyde concentration of 2%). Fixed PBMCs were collected by centrifugation (5 min, 500 xg, RT) and washed with ice cold DPBS. Cells were again collected (5 min, 500 xg, RT) and subsequently permeabilized by addition of 180 μL ice cold 100% methanol and incubation for 30 min on ice. Permeabilized PBMCs were washed twice with FACS buffer (DPBS, 2% heat-inactivated FBS, 2 mM EDTA) and stained with 50 L/well species-specific mastermixes for 30 min at 2-8° C. protected from light. Stained PBMCs were washed twice with ice cold FACS buffer, re-suspended in 100 μL FACS buffer and transferred to a 96-well U-bottom microplate.

Flow cytometric analysis was performed on a BD FACSCelesta™, and acquired data saved as flow cytometry standard (FCS) files. Data were analyzed with FlowJo software version 10.4. CD4⁺ T helper cells, CD4⁺ Treg cells, CD8⁺ T cells, and NK cells were gated, and the individual % pSTAT5⁺ cell fractions were plotted as a function of the supernatant dilution using GraphPad Prism software. The concentrations at which 50% of the maximum effect was observed (EC50) were calculated for each immune cell sub-set using a 4-parameter logarithmic fit.

The fold-changes in biological activity between mouse, cynomolgus monkey and human indicator immune cell subsets were calculated using EC50 values derived from the fitted dose-response curves (Table 4 andTable 5). For hIL7-hAlb the biological effect was increased by approximately 3.5-fold and 2.4-fold on cynomolgus monkey CD4*CD25″T helper cells and CD8⁺ T cells compared to human, whereas no difference in sensitivity between mouse and human was detected in both indicator subsets tested. hAlb-hIL2 was biologically active in equal measure on human, cynomolgus monkey and mouse CD4⁺CD25⁺ T_{regs}. Importantly, both hIL7-hAlb and hAlb-hIL2 were functional in all three species tested, hence identifying mouse and cynomolgus monkey as relevant species for in vivo pharmacology assessment.

TABLE 4
EC₅₀ values of hIL7-hAlb and hAlb-hIL2 on selected human,
cynomolgus monkey and mouse immune cell subset in vitro

| RiboCytokine | | CD4⁺ | CD4⁺ | |
|---|---|---|---|---|
| translated | Origin | CD25⁺ | CD25⁻ | CD8⁺ |
| protein | of PBMCs | Tregs | T cells | T cells |
| hIL7-hAlb | Human | | 0.21 | 0.29 |
| | Cynomolgus monkey | n.d. | 0.06 | 0.12 |
| | Mouse | | 0.24 | 0.28 |
| hAlb-hIL2 | Human | 0.01 | | |
| | Cynomolgus monkey | 0.01 | n.d. | n.d. |
| | Mouse | 0.01 | | |

EC50 values were determined per individual immune cell subset as % hIL7-hAlb and hAlb-hIL2-containing HEK293T/17 cell culture supernatants at which selected human, cynomolgus monkey and mouse immune cell subsets showed 50% of maximal STAT5 phosphorylation in vitro.
hAlb = human albumin, hIL = human interleukin, n.d. = not determined, PBMC = peripheral blood mononuclear cell,

TABLE 5
Species sensitivity to hIL7-hAlb and hAlb-hIL2-
mediated immune cell stimulating activity

| RiboCytokine | | CD4⁺ | CD4⁺ | |
|---|---|---|---|---|
| translated | Origin of | CD25⁺ | CD25⁻ | CD8⁺ |
| protein | PBMCs | T_{regs} | T cells | T cells |
| hIL7-hAlb | Cynomolgus monkey | n.d. | ≅3.5-fold | ≅2.4-fold |
| | Mouse | | Equal | Equal |
| hAlb-hIL2 | Cynomolgus monkey | Equal | n.d. | n.d. |
| | Mouse | Equal | | |

Species sensitivity is given as fold-increased potency on cynomolgus monkey and mouse versus human cytokine-specific indicator immune cell subsets assayed by STAT5 phosphorylation.
hAlb = human albumin, hIL = human interleukin, n.d. = not determined, PBMC = peripheral blood mononuclear cell,

### Example 3: Pharmacodynamics of BNT152 and BNT153 in Single-Dose Treated Naïve Mice

In order to investigate the activity of BNT152 and BNT153 in vivo, (i) cytokine receptor activation in T cell subsets was analyzed ex vivo by phosphorylated STAT5 flow cytometry in erythrocyte-lysed whole blood, and (ii) T cell activation status was monitored by assessing sCD25 levels in the serum via enzyme-linked immunosorbent assay (ELISA), as T cells are known to produce notable amounts of sCD25 following IL-2 stimulation (Pederson A E and Lauritsen J P, Scand J Immunol 2009 July; 70(1): 40-3). Cytokine receptor activation data was correlated with recorded hIL7-hAlb and hAlb-hIL2 levels in serum.

BALB/c mice were injected IV with 10 μg BNT152 or BNT153 (LNP-formulated RNA encoding hIL7-hAlb or hAlb-hIL2). LNP-formulated RNA encoding hAlb was used as control. Blood was sampled at 1, 4, 24, 48, 72, 96, 116, 140 and 164 h after injection, and serum was prepared.

The constituents of the LNPs, as well as their formulation and injection procedures are described in Example 1.

Blood collection was performed via the vena facialis. In brief, without prior anesthesia, mice were held tightly and using a lancet, the vena facialis was punctured in a precise and short movement. Blood was collected into an appropriate plastic tube, subsequently the restraining grip was loosened. Blood samples were centrifuged at 10,000×g and ambient temperature for 5 min and serum transferred to a pre-labeled 0.5 mL reagent tube, to be used subsequently for down-stream assays or storage at −20° C.

Phosphorylation of STAT5 was assessed as described in Example 2.

Serum cytokine concentrations were determined using V-PLEX Human hAlb-hIL2 and hIL7-hAlb kits custom-developed and measured at Meso Scale Diagnostics, LLC according to the manufacturer's instructions. Sera were diluted up to 800-fold depending on the expected cytokine concentration. Individual serum cytokine concentrations were calculated using recombinant hIL7-hAlb or hAlb-hIL2 as a standard. Soluble CD25 levels in serum were determined using the mouse CD25/IL2Ra DuoSet ELISA kit according to manufacturer's instructions.

For flow cytometry analyses, blood aliquots were treated with Lyse/Fix solution (BD) for 8 min at 37° C. and washed with DPBS. Cell pellets were resuspended in ice-cold methanol, incubated for >30 minutes at 2 to 8° C., and washed with ice-cold flow buffer (DPBS, 5% FCS, 5 mM EDTA). The cell pellets were then stained with ice cold a master mix containing a panel of antibodies diluted in flow buffer. After a 30 min incubation at 2 to 8° C. in the dark, the cells were resuspended in flow buffer, and stored at 2 to 8° C. until measurement.

Data were acquired on a BD FACSCelesta™ flow cytometer and analyzed with FlowJo software version 10.3.

Cytokine receptor activation in T cell subsets Serum levels of BNT152-translated hIL7-hAlb and BNT153-translated hAlb-hIL2 were detected as early as 1 h after injection, and peaked between 4 and 24 h after treatment (FIG. 5). The stimulatory effect of BNT152-translated hIL7-hAlb occurred instantly upon cytokine availability and was faster as well as initially much stronger than that of BNT153-translated hAlb-hIL2 (FIG. 5). hIL7-hAlb induced similar pSTAT5 levels in total CD4⁺, CD8⁺, and CD4⁺CD25 T_{H} cells, with an early decline evident at 6 h after injection followed by a rather stable phase in pSTAT5 levels until 72 h after injection, when hIL7-hAlb started disappearing from the blood. Phosphorylation of STAT5 in CD25⁺CD4⁺ T_{regs} followed a similar pattern but remained lower compared to the other T cell subsets.

In contrast to BNT152, where STAT5 phosphorylation correlated with serum availability of hIL7-hAlb after the peak phase, the levels of pSTAT5 increased up to 72 h in total CD4⁺ T cells. Particularly CD4⁺CD25⁺ T_{regs} profited from enhanced hAlb-hIL2 availability compared to CD+CD25 T_{H} cells, as expected and indicated by much higher levels of STAT5 phosphorylation throughout, and prolonged maintenance of the achieved phosphorylation level.

Of note, BNT152-translated hIL7-hAlb led to maintained high levels of STAT5 signaling in total CD8⁺ T cells until 72 h after injection, whereas BNT153-translated hAlb-hIL2 only initially stimulated phosphorylation of STAT5 in this T cell subset. Likewise, BNT152-translated hIL7-hAlb promoted STAT5 phosphorylation in CD4⁺CD25 T_{H} cells, while BNT153-translated hAlb-hIL2 hardly affected signaling in this T cell subset.

Serum levels of soluble CD25

BNT153 treatment led to elevated secretion of sCD25 compared to treatment with LNP-formulated hAlb RNA (FIG. 6). In particular, the highest sCD25 concentration in animals treated with BNT153 was measured 48 h post-injection and reached 12,800 pg/mL, approximately 27-fold above baseline values. The ability of BNT153-translated hAlb-hIL2 to trigger sCD25 secretion in the circulation is in line with known literature, which describes that mainly T_{regs} are able to shed large amounts of sCD25 upon activation (Pederson A E and Lauritsen J P, Scand J Immunol 2009 July; 70(1): 40-3, Lindqvist C A et al., Immunology. 2010; 131(3): 371-76).

### Example 4: Bioactivity of mIL7-mAlb LNP and BNT153 on Immune Cell Subsets in Mice

To determine the effect of BNT152 and BNT153 on circulating and lymphoid-tissue resident lymphocytes in vivo, naïve C57BL/6 mice (n=6 per group) were treated weekly for 3 weeks (Day 7, 14, and 21) with either the mouse surrogate mIL7-mAlb LNP, BNT153 or the combination of both. Albumin-encoding RNA formulated as LNP (hAlb) served as control.

In order to analyze the effect of the RiboCytokines on antigen-specific T cells, groups 5 to 8 received weekly doses of an RNA-LPX vaccine encoding a total of 20 tumor antigens on two ‘decatope’ RNAs (BL6_Deca1+2). The vaccine treatment started1 week before the first RiboCytokine dose (Day 0, 7, 14, and 21).

Peripheral blood was analyzed for immune cell subset composition on Day 14, 21, 28, and 35, and antigen-specific CD8⁺ T cells were quantified in the spleen on Day 35. The study design is depicted in FIG. 7.

The constituents of LNPs, as well as their formulation and injection procedures are described in Example 1.

RNA for vaccination was produced based on Kreiter et al. (Kreiter, S. et al. Cancer Immunol. Immunother. 56, 1577-87 (2007)) using a Beta-S-ARCA(D1) cap. RNA-LPX formulation was performed based on Kranz et al., Nature (2016). RNA-LPX was prepared at BioNTech under sterile and RNase-free conditions, i.e. all equipment was autoclaved and all surfaces cleaned with a cloth soaked in RNaseZAP® prior to use. A vial of RNA stock solution was thawed and consecutively diluted with water, 10 mM HEPES/0.1 mM EDTA, 1.5 M NaCl and L2 liposomes. The vial was vortexed immediately after each addition and incubated for 10 minutes at ambient temperature after all components were added.

Single cell suspensions from collected spleens were prepared according to a standard procedure. Spleens were mashed through 70 μm cell strainer using the plunger of a syringe to release the splenocytes into a tube. Cells were washed with an excess volume of PBS followed by centrifugation at 300×g for 6 minutes at ambient temperature and discarding the supernatants. Erythrocytes were lysed with erythrocyte lysis buffer (154 mM NH4Cl, 10 mM KHCO3, 0.1 mM EDTA) for 5 min at ambient temperature. The reaction was stopped with an excess volume of PBS. After another washing step, cells were resuspended in DC medium (RPMI medium1640 (1×)+GlutaMAX-I (Life Technologies), 10% FBS, 1% NEAA, 1% Na-pyruvat, 0.5% penicillin/streptomycin, 50 μM 2-Mercaptoethanol), passed through a 70 μm cell mesh again, counted according to SOP-010-028, and stored at 4° C. until further use. For flow cytometry analysis, 50 μL of blood collected from each mouse was transferred to a 96-well plate and stained with titrated amounts of antibodies. For the detection of antigen-specific T cells, the following MHC tetramers from MBL Life Science were added: Reps1 (Catalog No.: TB-5114-1), Adpgk (TB-5113-2), TRP2 (TB-5004-1), and Rpl18 (TBCM3-KBI-2). The extracellular staining procedure was carried out at 2-8° C. for 30 minutes. Afterwards, 200 μL of BD lysis buffer were added, mixed, and incubated for 6-8 minutes at ambient temperature in the dark. For intracellular staining, cells were washed once with 2 mL PBS (5 min, 460×g, ambient temperature) and fixed in 200 μL Fix/Perm buffer (Foxp3/Transcription Factor Staining Buffer Set, preparation according to the manufacturer's instructions) at 2-8° C. for 30 minutes. After centrifugation (5 min, 460×g, ambient temperature), cells were washed once with Perm buffer (Foxp3/Transcription Factor Staining Buffer Set) and stained with 50 L FoxP3 antibody solution at 2-8° C. for 30 minutes. Finally, cells were washed twice with Perm buffer (5 min, 460×g, RT) and cells were resuspended in 200 μL flow buffer (PBS supplemented with 5 mM EDTA and 5% FBS) supplemented with 33 μl counting beads (total volume per well: 233 μL). The samples were stored at 2-8° C. until measurement. Data were acquired on a BD FACSCelesta™ flow cytometer and analyzed with FlowJo software version 10.3 and GraphPad Prism 8. Analysis of immune cell subsets in the blood revealed a dominant increase of CD8⁺ T cells, CD4⁺ T cells and NK cells in mIL7-mAlb LNP (murine surrogate of BNT152 encoding mIL7-mAlb LNP) plus BNT153 treated animals (FIG. 8A-C). Expansion of CD4⁺ T cells was driven by mIL7-mAlb LNP, whereas both mIL7-mAlb LNP and BNT153 increased CD8⁺ T cell numbers leading to higher T cell numbers in the combination group. NK cell expansion depended solely on BNT153. BNT153 resulted in a transient increase of the Treg fraction among CD4⁺ T cells, which was prevented by addition of mIL7-mAlb LNP (FIG. 8D). Of note, the BNT153-mediated increase in Treg numbers in the blood was repeatedly observed to be normalized within 14 days (d) after initial treatment irrespective of a second RiboCytokine treatment at Day 7. Treg normalization was shown to be dose dependent and was observed at doses higher or equal to 3 μg per mouse (data not shown).

Tumor antigen-specific CD8⁺ T cell responses in the blood of RNA-LPX vaccinated mice were determined by major histocompatibility complex (MHC) class I tetramer staining and flow cytometry.

Three out of 4 analyzed antigen-specific T cell responses were significantly expanded by mIL7-mAlb LNP plus BNT153 co-treatment as compared to RNA-LPX vaccination alone (Adpgk=19-fold, Reps1=155-fold, TRP2=41-fold). Effects were weaker when mice were treated with either mIL7-mAlb LNP or BNT153 in combination with RNA-LPX vaccination (FIG. 9A). Similar effects were observed for the capacity of antigen-specific CD4⁺ and CD8⁺ T cells to secrete the effector cytokine IFNγ upon recognition of peptide antigen in an ELISpot assay. All tested antigen-specific CD4⁺ and CD8⁺ T cell responses were elevated in the mIL7-mAlb LNP plus BNT153 combination group. For most antigens, IFNγ release was weaker when either mIL7-mAlb LNP or BNT153 was administered in combination with vaccination (FIG. 9B). In summary, treatment of mIL7-mAlb LNP plus BNT153 strongly elevates CD4⁺ and CD8⁺ effector T cell responses over T_{regs}. When combined with RNA-LPX vaccination, RiboCytokine therapy potently elevates tumor antigen-specific T cell numbers and functionality.

### Example 5: mIL7-mAlb LNP Enhances CD25 Expression on Antigen-Specific CD8⁺ T Cells

IL-7 has been described to increase the expression of CD25 on T cells. We hypothetized that IL-7-mediated enhanced expression of CD25 on antigen-specific CD8⁺ T cells would render these T cells more susceptible to stimulation and expansion by IL-2. In order to demonstrate that BNT152 is able to increase CD25 expression especially on antigen-specific T cells, naïve C57BL/6 mice were immunized twice with 20 μg of an RNA-LPX vaccine encoding the neo-antigen Adpgk (Yadav et al., 2014, Nature 515, 572-576) on Day 0 and 7 to generate antigen-specific CD8⁺ T cells (groups 2-4; n=20 per group). On Day 14, groups 2 and 3 were treated with 3 μg mIL7-mAlb LNP or 3 μg hAlb LNP, in addition to the RNA-LPX vaccine. In order to evaluate the potency of mIL7-mAlb alone to stimulate CD25 expression, mice were treated with mIL7-mAlb alone without concomitant vaccination (group 4). Animals that received no treatment served to assess CD25 baseline expression on Day 14 (group 1; n=4). T cell subsets in the spleen were analyzed by flow cytometry 24, 48, 72 and 96 h after treatment on Day 14. The study design is depicted in FIG. 10. Single cell suspensions of splenocytes were prepared according to the standard procedure described in Example 4. For immunophenotyping, 2×10⁶ splenocytes/well were transferred to a 96-well U-bottom plate, centrifuged (3 min, 460×g, 2-8° C.), and the supernatants were discarded. Cells were stained with fixable viability dye in 200 μL PBS for 15 min at 2-8° C. in the dark. After washing once with 200 μL PBS (3 min, 460×g, 2-8° C.), cells were incubated with Adpgk-specific, H2-Db-restricted T-select tetramer (MBL Life Science; catalogue No. TB-5113-2) and standard monoclonal antibodies for CD8 and CD25 for 30 min at 2-8° C. After washing once with 200 μL PBS (3 min, 460×g, 2-8° C.), cells were resuspended in 200 μL flow buffer and stored at 2-8° C. until measurement. Data were acquired on a BD FACSymphony flow cytometer and analyzed with FlowJo software version 10.6.

RNA-LPX vaccine encoding Adpgk were prepared at BioNTech as described in Example 4.

The constituents of LNP RNA formulations, as well as their preparation and injection procedures are described in Example 1.

Treatment with mIL7-mAlb LNP clearly increased the fraction of CD25⁺ cells among antigen-specific CD8⁺ T cells in combination with the RNA-LPX vaccine compared to the RNA-LPX vaccine alone (FIG. 11A). Interestingly, mIL7-mAlb LNP treatment without concomitant RNA-LPX vaccination also raised the fraction of CD25⁺ cells among antigen-specific CD8⁺ T cells, albeit to a lower extent. In line with this observation, mIL7-mAlb LNP plus RNA-LPX vaccine led to a substantial increase in the expression level of CD25 on antigen-specific CD8⁺ T cells (FIG. 11B). A decline in the fraction of CD25⁺ antigen-specific CD8⁺ T cells, as well as their CD25 expression level, was observed from 72 h on, indicating that activated CD25⁺ T cells had started leaving the spleen and entering the circulation.

Treatment with mIL7-mAlb LNP substantially increased the fraction of CD25⁺ cells among CD4⁺ T cells as well as the expression of CD25, independent of the RNA-LPX vaccine (FIG. 11C, D). The RNA-LPX vaccine used here does not contain an MHC class Il-restricted epitope, and did not stimulate any CD25 upregulation by itself. CD25 expression levels were clearly raised by mIL7-mAlb treatment, with the RNA-LPX vaccine not exhibiting any additional effect (FIG. 11D).

In conclusion, mIL7-mAlb LNP was able to upregulate CD25 on antigen-specific CD8⁺ T cells, providing the basis for enhanced sensitivity to BNT153 and hence another mechanistic rationale for the combination of BNT152 with BNT153.

### Example 6: Therapeutic Efficacy of BNT152 and BNT153 in the CT26 and TC-1 Mouse Carcinoma models

Anti-tumor immunity and therapeutic activity of BNT152 and BNT153 were assessed in the s.c. mouse tumor models CT26 (BALB/c background) and TC-1 (C57BL/6 background).

BALB/c mice (n=11 per group) were inoculated with 5×10⁵ CT26 tumor cells on Day 0 and stratified according to tumor size on Day 10. Mice were vaccinated weekly for 4 weeks with an RNA-LPX vaccine encoding the tumor-specific antigen gp70, in combination with BNT152, BNT153 or the combination of both (Day 10, 17, 24, and 31). LNP-formulated RNA encoding hAlb only served as control. Anti-tumor activity and survival was monitored until Day 104. The study design is depicted inFigure 12.

CT26 murine tumor cells were cultured according to standard cell culture procedures. On Day 9 of the experiment, 19 d prior to the first immunization, CT26 tumor cells were harvested from a cell culture growing in log-phase (approx. 90% viability) and counted. The cell number was adjusted to 5×10⁶ cells/mL with PBS, and the cells were kept on ice until injection. Mice received a 100 μl s.c. injection into the upper flank corresponding to 5×10⁵ cells per mouse. TC-1 murine tumor cells (TC-1_luc_thyl-1) were ordered at TRON GmbH and cultured according to standard cell culture procedures. On Day 0 of the experiment, 12 d prior to the first vaccination, TC-1 tumor cells were harvested from a cell culture growing in log-phase (approx. 90% viability) and counted. The cell number was adjusted to 1×10⁶ cells/mL with PBS and the cells were kept on ice until injection. Mice received a 100 μl s.c. injection into the upper flank corresponding to 1×10⁵ cells per mouse.

RNA-LPX vaccines encoding gp70 or E7 were prepared at BioNTech as described in Example 4.

The constituents of LNP RNA formulations, as well as their preparation and injection procedures are described in Example 1.

In total, 79 mice were inoculated with tumor cells in order to ensure availability of 11 mice for each of the six groups with suitable tumor volumes at the start of the immunization regimen. Stratification of 66 animals was performed according to tumor size. The mean and median tumor sizes of the 66 animals included in the analysis were 16.2 mm3 and 12.5 mm3 at the start of the treatment, respectively.

Subcutaneous tumor growth was monitored by assessment of the tumor volume over time. To this end, the largest diameter ‘a’ and the smallest diameter ‘b’ were measured with a caliper every 2-4 d. Tumor volumes were calculated according to the following formula with the assumption that that the tumors are idealized ellipsoids: Tumor volume=(a [mm]×b [mm]2)/2.

Group median tumor volumes were calculated on each study day based on tumor volumes from live animals. Additionally, tumor volumes of animals euthanized due to tumor load were included according to the Last Observation Carried Forward (LOCF) principle, whereby tumor volumes from mice euthanized due to tumor burden remained part of the calculations.

For flow cytometry analysis, 50 μL of blood collected from each mouse was transferred to a round-bottom polystyrene tube and stained with E7 dextramer (Immudex; Catalog No.: JA2195-PE) for 10 minutes at 2 to 8° C. Subsequently, cells were stained with titrated amounts of antibodies at 2 to 8° C. for 30 minutes. Afterwards, 200 μL of BD lysis buffer were added, mixed, and incubated for 6 to 8 minutes at ambient temperature in the dark. Cells were then washed twice with 2 mL PBS (5 min, 460 xg, RT), resuspended in 200 μL buffer for intra-cellular staining, and the staining was carried out according to the manufacturer's instructions using the Foxp3/Transcription Factor Staining Buffer Set. At the end of the procedure, cells were resuspended in 200 μL flow buffer (500 mL DPBS, 5% FCS, 5 mM EDTA) supplemented with 33 μL CountBright™ Absolute Counting Beads. The samples were stored at 2 to 8° C. until measurement.

Data were acquired on a BD FACSCelesta™ flow cytometer and analyzed with FlowJo software version 10 and GraphPad Prism 8 Software (La Jolla, USA).

Anti-tumor activity was measured as tumor growth inhibition in the test groups compared to the control group and overall survival during an observation period of up to Day 104 after tumor inoculation. Treatment with BNT152 or BNT153 resulted in reduced tumor growth and prolonged survival compared to the control (FIG. 13). The combination of BNT152 plus BNT153 revealed superior anti-tumor potency with complete responses in 91% (10/11) of animals, while 18% (2/11) and 64% (7/11) of animals showed a complete response when treated with BNT152 or BNT153, respectively.

To confirm these findings in a second tumor model, C57BL/6 mice were inoculated with 1×10⁵ TC-1 tumor cells expressing the human papillomavirus 16 antigen E7 on Day 0 and stratified according to tumor size on Day 12. Mice were treated with either LNP-formulated RNA encoding hAlb, mIL7-mAlb LNP, BNT153 or mIL7-mAlb LNP plus BNT153 in combination with RNA-LPX vaccination encoding the viral tumor antigen E7 or irrelevant RNA-LPX control. Vaccination was administered 4 times (Day 12, 17, 24, and 31). Beginning 5 d after vaccination start, RiboCytokines were administered 3 times (Day 17, 24, and 31). Blood was sampled on Day 24 and 31 for immunophenotyping studies by flow cytometry. Anti-tumor activity and survival was monitored until Day 112. The study design is depicted in FIG. 14.

As observed in the CT26 colon carcinoma model (FIG. 13), simultaneous treatment with mIL7-mAlb LNP, BNT153 and RNA-LPX vaccination resulted in tumor shrinkage and long-term survival in a considerable fraction of mice. Approximately half (7/15) of the mice receiving the triple combination experienced a complete response. With TC-1 being a weakly immunogenic (‘cold’) tumor without the presence of a pre-existing T cell response, RiboCytokine treatment was not effective without RNA-LPX vaccination. Complete responses were neither observed in groups treated with mIL7-mAlb LNP plus BNT153, nor when RiboCytokines were individually combined with RNA-LPX vaccination, despite temporary tumor control and survival benefit in these groups (FIG. 15). Of note, significant therapeutic activity of BNT153 without RNA-LPX vaccination was observed in additional therapeutic tumor experiments in the CT26 mouse colon carcinoma and B16F10 mouse melanoma models (data not shown).

Analysis of blood samples of mice linked the significant therapeutic benefit in the triple combination group shown in FIG. 15 to a strong elevation of E7 tumor antigen-specific CD8⁺ T cells (FIG. 16A). As observed in naïve mice (FIG. 9), mIL7-mAlb LNP was able to reduce the elevated Treg fraction induced by BNT153 (FIG. 16B), resulting in an approximately 2,000-fold increase of the E7-specific T cell to Treg ratio (FIG. 16C).

### Example 7: Pharmacodynamics of BNT152 and BNT153 in the Biomarker Study in Cynomolgus Monkey

In order to investigate the activity of BNT152 and BNT153 in cynomolgus monkeys, (i) lymphocyte counts as well as the T cell subset and NK cell numbers were analyzed by flow cytometry, and (ii) sCD25 levels in the serum, which served as a surrogate marker for lymphocyte activation, were determined by ELISA. Cynomolgus monkeys were injected IV with 60 or 300 μg/kg BNT152 or 60 or 180 μg/kg BNT153 on Days 1 and 22. As a control, animals were treated with empty LNPs (i.e., without RNA payload), at a lipid dose comparable to 120 μg/kg. Blood samples for lymphocyte enumeration and immunophenotyping were taken pre-dose, as well as on Days 8, 21 and 29. Serum samples for sCD25 level assessment were taken pre-dose, as well as on Days 2, 4, 6, 8, 21, 23, 25, 27, and 29.

The constituents of LNPs, as well as their formulation and injection procedures are described in Example 1.

At least 2 mL whole blood per animal per sampling time were withdrawn from the vena cephalica or vena saphena magna of all animals and collected into Li-heparin collection tubes.

Peripheral blood mononuclear cells (PBMC) were isolated by density centrifugation using Histopaque (Sigma). The PBMCs were washed twice medium (RPMI 1640, Invitrogen) supplemented with 10% heat inactivated fetal calf serum (FCS), 1 mM sodium pyruvate (Sigma), 100.000 IU/L penicillin, 100 mg/L streptomycin (Invitrogen), 5 mg/L gentamicin (Sigma), 25 mM HEPES buffer, 2 mM alpha-glutamin and 5×10⁻⁵ M 2-mercaptoethanol. After suspending the cells in stain buffer (FBS, BD Cat. No. 554656) cell quantification was performed using XP-300 (Sysmex) and the concentration of the cells were adjusted to 10×10⁶ cells/mL. After surface staining, fixation and perforation of the cells (Human FoxP3 Buffer Set, BD cat. No. 560098) an intracellular FoxP3 staining (BD Biosciences, Cat. No. 560047) was carried out. The determination of regulatory T lymphocytes was performed using Cytomics FC 500 (Beckmann Coulter GmbH, 47704 Krefeld, Germany).

Levels of sCD25 were determined using the Human CD25/IL-2R alpha Quantikine ELISA Kit following the manufacturer's protocol. Briefly, 1:5 dilutions were prepared by mixing 20 μL serum and 80 μL Calibrator diluent RD6S (Group 1) or 1:10 dilutions by mixing 10 μL serum and 90 μL Calibrator diluent RD6S (Groups 2-7). For the general procedure 100 μL Assay Diluent RD-1, 50 μL standard/sample diluted in RD6S and 100 μL human IL-2Ra conjugated to horseradish peroxidase were mixed carefully and incubated for 3 h at RT. Next, the plate was washed three times with 300 μL wash buffer per well and wash step. Next, 200 μL substrate solution was added and the plate incubated in the dark. 50 μl stop solution was added after sufficient blue coloration and the plate was measured with an absorption of 450 nm wavelength using a microplate reader.

The absolute numbers of lymphocytes were decreased 24 h after the first and second dose with BNT152 or BNT513 at both tested dose levels, but not for animals treated with empty LNPs (FIG. 17). At 5 to 7 d after each administration, the lymphocyte counts were increased up to 3.1-fold over pre-dose levels for all dose levels except the 60 μg/kg dose of BNT152. The increase in lymphocyte counts was followed by a consecutive drop to normal values within 10 to 12 d. The pharmacodynamic (PD) profile of the lymphocyte compartment as a whole was therefore consistent with mouse studies, in which immediate recruitment of lymphocytes to lymphoid organs and consecutive systemic proliferation of T cells were observed.

Absolute numbers of T cell subsets and NK cells as well as the relative abundance of T_{regs} were analyzed by flow cytometry at pre-dose and on Days 8, 21 and 29. Overall, the changes in lymphocyte counts after BNT152 and BNT153 administration were reflected by CD8⁺ T cell and NK cell numbers (FIG. 18). A strong increase in numbers was recorded in all groups, except the group treated with 60 μg/kg BNT152, on Day 8 and Day 29 of the study, whereas on Day 21 (prior to the second dose), the numbers were back to baseline values. For CD8⁺ T cells, the increase was up to 5.6-fold over pre-dose values. The relative abundance of T_{regs} were strongly increased on Days 8 and 29 in BNT153-treated animals, leading to a reduced CD8⁺ T cell to Treg ratio. On the other hand, Treg proportions were less affected in animals treated with BNT152. Treatment with 60 μg/kg and 300 μg/kg BNT152 or 180 μg/kg BNT153 led to an increase in NK cell numbers. Moreover, the PD profiles of CD8⁺ T cells and NK cells were consistent with mouse studies, in which immediate recruitment of lymphocytes to lymphoid organs and consecutive systemic proliferation of T cells were observed.

Serum concentrations of sCD25 were strongly increased 2 to 4 d after administration of BNT153 (FIG. 19). The highest sCD25 concentrations measured after 60 and 180 μg/kg BNT153 were 8 ng/ml (4.4-fold over pre-dose levels) and 24.2 ng/ml (26-fold) on average, respectively. BNT152 induced only moderately elevated sCD25 levels. Serum sCD25 concentrations subsequently declined to levels comparable to those measured in empty LNP-treated animals on Day 21 (pre-dose Cycle 2). Following the second RiboCytokine dosing, sCD25 levels increased with similar kinetics, but with lower peak levels in all groups. Peak levels were detected 2 to 4 d after the second dosing and were comparable to those measured after the first dosing in animals treated with 60 μg/kg RiboCytokines. In contrast, peak sCD25 levels in the sera of animals receiving 180 g/kg BNT153 were reduced 2.8-fold compared to the first dosing.

### Example 8: Ex Vivo Cytokine Release by BNT152 or BNT53 in Human Whole Blood

To address potential immune activation as a result of direct contact of BNT152 or BNT153 with PBMCs, cytokine release after incubation of heparinized human blood with both drug products was assessed. Venous whole blood was collected from seven healthy volunteers in sterile syringes the same day as the cytokine release assay (CRA) was set up. Heparin was used as anti-coagulant. The CRA was performed for all seven blood donors in parallel, but on separate plates. After collection of heparinized whole blood and preparation of all test and control items (spiking solutions), 190 μL of WB was seeded in each well of a 96-well-plate. Subsequently, 10 μL of each spiking solution were added to WB, leading to a final volume of 200 UL and an additional 1:20 dilution of samples. For each sample assay duplicates were generated, meaning for each spiking solution and each donor two wells/sample. For each blood donor a separate 96-well-plate was used. Finally, the plates were incubated at 37° C. and 5% CO₂. After 24 h incubation, the plates were centrifuged at 500×g for 5 min. The plasma of all samples was harvested, transferred to a new 96-well-plate and stored at-15° C. to −25° C. until performing the CBA (see below) for at least 3 h. The Cytometric Bead Array (CBA) assay was performed with the thawed plasma samples according to the manufacturer's instructions for ProCarta multiplex kits. For evaluation of cytokine concentrations, samples were measured by using the Bio-Plex 200 System. Graphical analysis was performed using GraphPAd Prism 6.

The final BNT152 or BNT153 in-assay concentrations were 0.000064, 0.00032, 0.0016, 0.008, 0.041, 0.2 and 1 μg/mL. These concentrations cover doses of 0.005 to 71 μg/kg assuming a patient with 70 kg body weight and a total blood volume of 5 L. 10 UM Resiquimod (R848) was used as positive control, which is a small molecule (TLR7 agonist) known to induce secretion of several (pro-) inflammatory cytokines in human whole blood. Empty LNPs without RNA payload were used as negative control; the lipid dose was adjusted to 1 μg/mL BNT152/BNT153.

No drug product mediated release of pro-inflammatory cytokines (IFNα, IFNγ, IL-1B, IL-2, IL-12p70, IL-6, IL-8, IP-10 or TNFa) was detected after BNT152 or BNT153 incubation.

### Example 9: Gen-LNPs are Suitable for Obtaining Strong RiboCytokine Activity

To identify an RNA formulation ensuring optimal systemic availability and immunostimulatory potency of RiboCytokines, multiple delivery vehicles were compared. In a series of experiments, naïve BALB/c mice received albumin-IL2-encoding RNA formulated with either Gen-LNPs (Genevant Sciences Corporation, Example 1), Psar-23-LNPs, NI-LNP1, NI-LNP6 pH6, DLP14-LPX, P8-LNPs, F12-LPX (BioNTech RNA Pharmaceuticals) or TransIT (Mirus Bio, Example 1). Apart from the experiment depicted in FIG. 20C, all animals were co-treated with a gp70-encoding RNA-LPX vaccine (see also Example 4). In one experiment, mice also received hIL7-hAlb LNP (FIG. 20A, B; FIG. 21A). Data shown in FIGS. 21E and H were generated in an experiment where mice were treated with a combination of mAlb fused to mouse IL-2 (mAlb-mIL2) and anti-PD-L1 antibody. All RNA formulations were administered as described in Example 1.

Cytokine concentrations in serum samples were determined using the V-PLEX Human IL-2 Kit, the V-PLEX Human IL-7 Kit and the MSD® Multi-Spot Assay System (Meso Scale Discovery) following the manufacturer's protocol. Briefly, the assay plate was equilibrated by washing with 150 μL PBS. Custom recombinant albumin-cytokine fusion constructs (hIL7-hAlb and hAlb-hIL2) were used as standards. Standards (1:4 serial dilutions in sample diluent) and diluted cynomolgus macaque serum samples (1:2, 1:10, and 1:80 dilutions in 50 μl sample diluent) were added to the equilibrated plate and incubated for 2 h at ambient temperature with constant shaking. The plate was washed three times with 150 μL PBS, and 25 μL of detection antibodies (diluted 1:50 in antibody diluent) were added, followed by incubation for 2 h at ambient temperature with constant shaking. The plate was washed three times with 150 μL PBS, 150 μL of 2×Read Buffer was added, and the plate was immediately analyzed on a MESO QuickPlex SQ 120 imager (Meso Scale Discovery).

The frequencies and numbers of gp70-specific cells were analyzed by flow cytometry using a T-select MHC Tetramer (MBL Life Science; catalogue No. TS-M521-1) and additional antibodies, following the standard protocol described in Example 4.

Administration of Gen-LNP-formulated hAlb-hIL2-encoding RNA alone or with hIL7-hAlb RNA led to more than 7-fold higher serum levels of translated RiboCytokines, compared to both Psar-23-LNPs and P8-LNPs (FIG. 20A-C). In animals treated with NI-LNP1, NI-LNP6 pH6, DLP14-LPX formulated RNA, only minimal levels of translated fusion protein were detected.

In line with these data, treatment with Gen-LNP formulated RNA led to the largest increase in gp70-specific CD8⁺ T cells (FIG. 21A, B). Gen-LNP-treated animals had approximately 2- or 3-fold greater numbers of gp70-reactive CD8⁺ T cells than mice that received P8-LNPs or Psar-23-LNPs, respectively. Similar trends were observed when comparing gp70-specific T cell responses boosted by Gen-LNP-encoded IL-2 as compared to RNA formulated with TransIT and F12-LPX (FIG. 21D, E, G, H). The contrast was particularly sharp seven days after the initial treatment, when the proportion of gp70-reactive cells in the CD8⁺ T cell compartment in mice treated with Gen-LNPs reached approximately 30% (FIG. 21C). In animals that received TransIT or F12-LPX formulated RNA, the respective frequencies remained below 2% (FIG. 21D, E). On Day 14, animals treated with Gen-LNP-formulated RNA exhibited a substantially stronger expansion of gp70-specific CD8⁺ T cells (FIG. 21F) than animals that received TransIT- or F12-LPX-formulated RNA (FIG. 21G, H).

Collectively, these data demonstrate that the Gen-LNP formulation is suitable for obtaining the beneficial pharmacodynamic and phamacokinetc properties of RiboCytokines. Gen-LNPs, compared to other tested formulations, maximized the systemic availability of RiboCytokine RNA-encoded proteins, leading to potent enhancement of antigen-specific T cell responses.

### Example 10: BNT152 Rather than BNT153 Expands CD8⁺ T Cells with Specificities Other than the Vaccine—Encoded Antigen, which is Boosted by the Combination of the Two

Superior therapeutic anti-tumor activity of BNT152 (mouse surrogate mIL7-mAlb LNP) plus BNT153 in combination with an RNA-LPX vaccine compared to BNT152 (mouse surrogate) plus BNT153 or the combination of either of the two with an RNA_LPX vaccine was demonstrated in the weakly immunogenic (‘cold’) tumor model TC-1 (Example 6). Strong E7 tumor antigen-specific CD8⁺ T cells were observed exclusively in response to treatment with the triple combination (FIG. 16A).

Inhibited tumor growth and shrinkage of tumors in response to this treatment (FIG. 15) implies that tumor cells must have been destroyed, a process presumably driven by the therapy-induced antigen-specific CD8⁺ T cells. Tumor cell lysis by therapy-induced tumor-specific CD8⁺ T cells recognizing their target antigen on the tumor cells can lead to the release of antigens other than the vaccine antigen. These antigens can potentially prime CD8⁺ T cells with specificities for antigens other than the vaccine antigen, which increases the breadth of the anti-tumor T cell response and decreases the likelihood of tumor immune escape by outgrowth of (vaccine) antigen loss variants.

Treatment with BNT153 in combination with the RNA-LPX vaccine primarily induced vaccine antigen-specific, i.e. E7-specific CD8⁺ T cells, and non-E7-specific CD8⁺ T cells were induced ˜3-fold over those in mice neither treated with RiboCytokines nor an E7 RNA-LPX vaccine (FIG. 22). In contrast, the combination of mIL7-mAlb LNP with RNA-LPX vaccine induced non-E7-specific CD8⁺ T cells more than 12-fold over those in mice neither treated with RiboCytokines nor an E7 RNA-LPX vaccine. The combination of mIL7-mAlb LNP plus BNT153 was able to enhance the induction of non-vaccine-specific CD8⁺ T cells beyond that of either mIL7-mAlb LNP or BNT153 alone, to more than 23-fold.

These findings demonstrate that the combination of mIL7-mAlb LNP and BNT153 with an RNA-LPX vaccine not only induces vaccine-antigen-specific CD8⁺ T cells but also leads to the induction of CD8⁺ T cells specific for antigens other than the vaccine antigen, and thus broadens the anti-tumor CD8⁺ T cell repertoire.

### Example 11: BNT152 Plus BNT153 Strongly Expands and Maintains the Antigen-Specific T Cell Memory Pool

IL-2 has been reported to emphasize T cell differentiation into effector T cells. Such effector T cells are often short-lived and believed to hardly contribute to the generation of tumor-specific T cell memory. IL-7, on the other hand, has been known to support memory formation and survival of memory T cells. In order to evaluate the potential of BNT152 plus BNT153 to generate antigen-specific T cell memory, we treated naïve BALB/c mice (n=5 per group) weekly for 3 weeks (Day 0, 7, and 14) with either the combination of the BNT152 mouse surrogate mIL7-mAlb LNP plus BNT153 together with an RNA-LPX vaccine encoding the tumor antigen gp70, or with RNA-LPX vaccine alone. Peripheral blood was analyzed for immune cell subset composition on Day 21 (priming phase) and on Day 56 and 358 (memory). The frequencies and numbers of gp70-specific cells were analyzed by flow cytometry using a T-select MHC Tetramer (MBL Life Science; catalogue No. TS-M521-1) and additional antibodies, following the standard protocol described in Example 4.

RNA-LPX vaccine encoding gp70 was prepared at BioNTech as described in Example 4.

The constituents of LNP RNA formulations, as well as their preparation and injection procedures are described in Example 1.

As observed before in Example 4, the combination of mIL7-mAlb and BNT153 enhanced the expansion of antigen-specific CD8⁺ T cells by several folds of magnitude after three treatments, compared to the RNA-LPX vaccine alone (42.4 vs 6.6% of total CD8⁺ T cells in the blood; Day 21; FIG. 23A). After 42 days without treatment, antigen-specific CD8⁺ T cells remained similarly high as directly after priming and expansion as measured on Day 21 (48.8 vs 10.0%; Day 56). After another 302 days (Day 358; 344 days after the last vaccination), antigen-specific CD8⁺ T cells had contracted and represented a decreased fraction of total CD8⁺ T cells in the blood, as expected to occur during T cell contraction and memory development. Interestingly, the group treated with mIL7-mAlb plus BNT153 still boasted a fraction of antigen-specific CD8⁺ T cells of almost 19%, compared to 4% in the RNA-LPX vaccine alone group. This finding demonstrates that the combination treatment of mIL7-mAlb plus BNT153 does not hamper memory T cell formation, but instead enhances the size of the memory pool, and maintains a substantional fraction of antigen-specific CD8⁺ T cells for at least a year. Consistent with this finding, a higher fraction of antigen-specific CD8⁺ T cells displayed a memory phenotype of high CD127 (IL-7 receptor a) expression and high or low KLRG1 expression in the combination group compared to the RNA-LPX vaccine alone group on Day 56 as well as on Day 358 (FIG. 23B).

In summary, the combination of mIL7-mAlb and BNT153 strongly supports not only the priming and expansion of antigen-specific CD8⁺ T cells, but also promotes their proper memory conversion and enhances longevity of the antigen-specific CD8⁺ T cell response.

### Example 12: Treatment with BNT152 Plus BNT153 in Combination with an RNA-LPX Vaccine Enables Anti-Tumor Immunity Against Tumor Cells not Expressing the Vaccine Antigen Upon Tumor Rechallenge

It was observed that the combination of BNT152 plus BNT153 is able to expand not only vaccine-antigen specific CD8⁺ T cells but also T cells with other specificities, presumably due to tumor cell lysis by therapy-induced tumor-specific CD8⁺ T cells and subsequent antigen release (Example 10). In order to demonstrate that BNT152 plus BNT153 are able to induce tumor-specific CD8⁺ T cells with specificities other than the vaccine antigen that are able to recognize and eliminate tumor cells, the following study was performed.

BALB/c mice (n=11 per group) were inoculated s.c. with 5×10⁵ syngeneic CT26 wildtype (WT) tumor cells on Day 0 and stratified according to tumor size on Day 13. Mice were vaccinated weekly for six weeks with an RNA-LPX vaccine encoding the tumor-specific antigen gp70 and anti-PD-L1 antibody (Day 13, 19, 27, 34, 41 and 48), in combination with the BNT152 mouse surrogate mIL7-mAlb LNP, the BNT153 mouse surrogate mAlb-mIL2 or the combination of both (Day 15, 22, 29, 36, 43 and 50). RNA-LPX vaccine plus anti-PD-L1 antibody in combination with LNP-formulated RNA encoding mAlb served as control. Surviving mice in the quadruple combination group were rechallenged with 5×10⁵ CT26 tumor cells either expressing the tumor antigen gp70 (CT26 WT; n=4) or not (CT26 gp70ko; n=5) on Day 133. Untreated BALB/c mice inoculated with either tumor cell line served as controls (n=5 per group). Anti-tumor activity and survival was monitored for 28 days (until Day 161).

CT26 murine tumor cells were cultured according to standard cell culture procedures, and mice received a 100 μL s.c. injection into the upper flank corresponding to 5×10⁵ cells per mouse as described in Example 6. Subcutaneous tumor growth was monitored by assessment of the tumor volume over time as described in Example 6. Anti-tumor activity was measured as overall survival during an observation period of up to Day 110 after the first tumor inoculation. Median group tumor volumes were calcuted to visualize tumor growth after tumor rechallenge.

RNA-LPX vaccine encoding gp70 was prepared at BioNTech as described in Example 4.

Mouse surrogates mIL7-mAlb and mAlb-mIL2 were formulated with LNPs (TransIT, Mirus Bio) as described in Example 1.

Peripheral blood was analyzed for immune cell subset composition on Day 34. The frequencies and numbers of gp70-specific cells were analyzed by flow cytometry using a T-select MHC Tetramer (MBL Life Science; catalogue No. TS-M521-1) and additional antibodies, following the standard protocol described in Example 4.

Similar to the findings described in Example 6, mIL7-mAlb and mAlb-mIL2 each improved the survival of mice compared to the control group (two complete responses), and mAlb-mIL2 was again superior to mIL7-mAlb, with six compared to three complete responses (FIG. 24A). The combination of the two boosted anti-tumor activity and led to a complete response in all mice. In line with these results, analysis of tumor antigen-specific CD8⁺ T cells seven days after the third vaccination (Day 34) revealed that 64% of all circulating CD8⁺ T cells were specific against the tumor antigen in the group treated with mIL7-mAlb plus mAlb-mIL2 in combination with RNA-LPX vaccine plus anti-PD-L1 antibody, compared to 29% in the control group that received RNA-LPX vaccine plus anti-PD-L1 antibody only (FIG. 24B).

In order to assess whether the induced T cell were able to reject tumors no longer bearing the vaccine-encoded antigen, mice that had been treated with the combination of mIL7-mAlb and mAlb-mIL2 together with RNA-LPX vaccine and anti-PD-L1 antibody were rechallenged with CT26 gp70ko tumor cells on Day 133, and their anti-tumor response compared to mice treated the same but rechallenged with CT26 WT tumor cells.

Untreated mice inoculated with either tumor cell line developed progressively growing tumors as expected (FIG. 24C). In contrast, mice that had been treated with mIL7-mAlb and mAlb-mIL2 together with RNA-LPX vaccine and anti-PD-L1 antibody prevented the growth of all CT26 WT tumors, indicating strong tumor-specific T cell memory induced by the treatment. Interestingly, mice that had been treated with mIL7-mAlb and mAlb-mIL2 together with RNA-LPX vaccine and anti-PD-L1 antibody challenged with tumor cells that did not express the vaccine antigen gp70 were equally able to fully prevent tumor growth. This finding indicates that besides vaccine-antigen specific CD8⁺ T cells, other cells, presumably T cells with other specificities, must have been induced by the quadruple treatment that enabled these mice to reject tumors devoid of the vaccine antigen. It is assumed that tumor cell killing by vaccine-antigen specific CD8⁺ T cells led to tumor antigen release and the priming of new T cell specificities, whose expansion and survival was dependent on the co-treatment with mIL7-mAlb and mAlb-mIL2.

1. A composition or medical preparation comprising at least one RNA, wherein the at least one RNA encodes:
(i) an amino acid sequence comprising human IL7 (hIL7), a functional variant thereof, or a functional fragment of the hIL7 or the functional variant thereof; and/or
(ii) an amino acid sequence comprising human IL2 (hIL2), a functional variant thereof, or a functional fragment of the hIL2 or the functional variant thereof.

2. The composition or medical preparation of claim 1, wherein the amino acid sequence under (i) comprises human albumin (hAlb), a functional variant thereof, or a functional fragment of the hAlb or the functional variant thereof.

3. The composition or medical preparation of claim 2, wherein the hAlb, the functional variant thereof, or the functional fragment of the hAlb or the functional variant thereof is fused with the hIL7, the functional variant thereof, or the functional fragment of the hIL7 or the functional variant thereof.

4. The composition or medical preparation of claim 3, wherein the hAlb, the functional variant thereof, or the functional fragment of the hAlb or the functional variant thereof is fused to the C-terminus of the hIL7, the functional variant thereof, or the functional fragment of the hIL7 or the functional variant thereof.

5. The composition or medical preparation of any one of claims 1 to 4, wherein the amino acid sequence under (ii) comprises human albumin (hAlb), a functional variant thereof, or a functional fragment of the hAlb or the functional variant thereof.

6. The composition or medical preparation of claim 5, wherein the hAlb, the functional variant thereof, or the functional fragment of the hAlb or the functional variant thereof is fused with the hIL2, the functional variant thereof, or the functional fragment of the hIL2 or the functional variant thereof.

7. The composition or medical preparation of claim 6, wherein the hAlb, the functional variant thereof, or the functional fragment of the hAlb or the functional variant thereof is fused to the N-terminus of the hIL2, the functional variant thereof, or the functional fragment of the hIL2 or the functional variant thereof.

8. The composition or medical preparation of any one of claims 1 to 7, wherein each of the amino acid sequences under (i), or (ii) is encoded by a separate RNA.

9. The composition or medical preparation of any one of claims 1 to 8, wherein
(i) the RNA encoding the amino acid sequence under (i) comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5; and/or
(ii) the amino acid sequence under (i) comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4.

10. The composition or medical preparation of any one of claims 1 to 9, wherein
(i) the RNA encoding the amino acid sequence under (ii) comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
(ii) the amino acid sequence under (ii) comprises the amino acid sequence of SEQ ID NO: 6, or an amino acid sequence having at least 99%, 98%, 97%, 96%, 95%, 90%, 85%, or 80% identity to the amino acid sequence of SEQ ID NO: 6.

11. The composition or medical preparation of any one of claims 1 to 10, wherein at least one of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

12. The composition or medical preparation of any one of claims 1 to 11, wherein each of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

13. The composition or medical preparation of any one of claims 1 to 12, wherein at least one RNA comprises the 5′ cap m₂^{7,3′-O}Gppp(m₁^{2′-O)ApG.}

14. The composition or medical preparation of any one of claims 1 to 13, wherein each RNA comprises the 5′ cap m₂^{7,3′-O}Gppp(m₁^{2′-O)ApG.}

15. The composition or medical preparation of any one of claims 1 to 14, wherein at least one RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

16. The composition or medical preparation of any one of claims 1 to 15, wherein each RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

17. The composition or medical preparation of any one of claims 1 to 16, wherein at least one RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

18. The composition or medical preparation of any one of claims 1 to 17, wherein each RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

19. The composition or medical preparation of any one of claims 1 to 18, wherein at least one RNA comprises a poly-A sequence.

20. The composition or medical preparation of any one of claims 1 to 19, wherein each RNA comprises a poly-A sequence.

21. The composition or medical preparation of claim 19 or 20, wherein the poly-A sequence comprises at least 100 nucleotides.

22. The composition or medical preparation of any one of claims 19 to 21, wherein the poly-A sequence comprises or consists of the nucleotide sequence of SEQ ID NO: 15.

23. The composition or medical preparation of any one of claims 1 to 22, wherein the RNA is formulated as a liquid, formulated as a solid, or a combination thereof.

24. The composition or medical preparation of any one of claims 1 to 23, wherein the RNA is formulated for injection.

25. The composition or medical preparation of any one of claims 1 to 24, wherein the RNA is formulated for intravenous administration.

26. The composition or medical preparation of any one of claims 1 to 25, wherein the RNA is formulated or is to be formulated as lipid particles.

27. The composition or medical preparation of claim 26, wherein the RNA lipid particles are lipid nanoparticles (LNP).

28. The composition or medical preparation of any one of claims 1 to 27, which is a pharmaceutical composition.

29. The composition or medical preparation of claim 28, wherein the pharmaceutical composition further comprises one or more pharmaceutically acceptable carriers, diluents and/or excipients.

30. The composition or medical preparation of any one of claims 1 to 27, wherein the medical preparation is a kit.

31. The composition or medical preparation of claim 30, wherein the RNA encoding the amino acid sequence under (i) and the RNA encoding the amino acid sequence under (ii) are in separate vials.

32. The composition or medical preparation of claim 30 or 31, further comprising instructions for use of the RNAs for treating or preventing cancer.

33. The composition or medical preparation of any one of claims 1 to 32 for pharmaceutical use.

34. The composition or medical preparation of claim 33, wherein the pharmaceutical use comprises a therapeutic or prophylactic treatment of a disease or disorder.

35. The composition or medical preparation of claim 34, wherein the therapeutic or prophylactic treatment of a disease or disorder comprises treating or preventing cancer.

36. The composition or medical preparation of any one of claims 1 to 35, which is for administration to a human.

37. A method of treating cancer in a subject comprising administering at least one RNA to the subject, wherein the at least one RNA encodes:
(i) an amino acid sequence comprising human IL7 (hIL7), a functional variant thereof, or a functional fragment of the hIL7 or the functional variant thereof; and/or
(ii) an amino acid sequence comprising human IL2 (hIL2), an functional variant thereof, or a functional fragment of the hIL2 or the functional variant thereof.

38. The method of claim 37, wherein the amino acid sequence under (i) comprises human albumin (hAlb), a functional variant thereof, or a functional fragment of the hAlb or the functional variant thereof.

39. The method of claim 38, wherein the hAlb, the functional variant thereof, or the functional fragment of the hAlb or the functional variant thereof is fused with the hIL7, the functional variant thereof, or the functional fragment of the hIL7 or the functional variant thereof.

40. The method of claim 39, wherein the hAlb, the functional variant thereof, or the functional fragment of the hAlb or the functional variant thereof is fused to the C-terminus of the hIL7, the functional variant thereof, or the functional fragment of the hIL7 or the functional variant thereof.

41. The method of any one of claims 37 to 40, wherein the amino acid sequence under (ii) comprises human albumin (hAlb), a functional variant thereof, or a functional fragment of the hAlb or the functional variant thereof.

42. The method of claim 41, wherein the hAlb, the functional variant thereof, or the functional fragment of the hAlb or the functional variant thereof is fused with the hIL2, the functional variant thereof, or the functional fragment of the hIL2 or the functional variant thereof.

43. The method of claim 42, wherein the hAlb, the functional variant thereof, or the functional fragment of the hAlb or the functional variant thereof is fused to the N-terminus of the hIL2, the functional variant thereof, or the functional fragment of the hIL2 or the functional variant thereof.

44. The method of any one of claims 37 to 43, wherein each of the amino acid sequences under (i), or (ii) is encoded by a separate RNA.

45. The method of any one of claims 37 to 44, wherein
(i) the RNA encoding the amino acid sequence under (i) comprises the nucleotide sequence of SEQ ID NO: 5, or a nucleotide sequence having at least 99%, 98%, 97%, 96%, 95%, 90%, 85%, or 80% identity to the nucleotide sequence of SEQ ID NO: 5; and/or
(ii) the amino acid sequence under (i) comprises the amino acid sequence of SEQ ID NO: 4, or an amino acid sequence having at least 99%, 98%, 97%, 96%, 95%, 90%, 85%, or 80% identity to the amino acid sequence of SEQ ID NO: 4.

46. The method of any one of claims 37 to 45, wherein
(i) the RNA encoding the amino acid sequence under (ii) comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
(ii) the amino acid sequence under (ii) comprises the amino acid sequence of SEQ ID NO: 6, or an amino acid sequence having at least 99%, 98%, 97%, 96%, 95%, 90%, 85%, or 80% identity to the amino acid sequence of SEQ ID NO: 6.

47. The method of any one of claims 37 to 46, wherein at least one of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

48. The method of any one of claims 37 to 47, wherein each of the amino acid sequences under (i), or (ii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

49. The method of any one of claims 37 to 48, wherein at least one RNA comprises the 5′ cap m₂^{7,3′-O}Gppp(m₁^{2′-O})ApG.

50. The method of any one of claims 37 to 49, wherein each RNA comprises the 5′ cap m₂^{7,3′-O}Gppp(m₁^{2′-O)ApG.}

51. The method of any one of claims 37 to 50, wherein at least one RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

52. The method of any one of claims 37 to 51, wherein each RNA comprises a 5′ UTR comprising the nucleotide sequence of SEQ ID NO: 13, or a nucleotide sequence having at least 99%, 98%, 97%, 96%, 95%, 90%, 85%, or 80% identity to the nucleotide sequence of SEQ ID NO: 13.

53. The method of any one of claims 37 to 52, wherein at least one RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

54. The method of any one of claims 37 to 53, wherein each RNA comprises a 3′ UTR comprising the nucleotide sequence of SEQ ID NO: 14, or a nucleotide sequence having at least 99%, 98%, 97%, 96%, 95%, 90%, 85%, or 80% identity to the nucleotide sequence of SEQ ID NO: 14.

55. The method of any one of claims 37 to 54, wherein at least one RNA comprises a poly-A sequence.

56. The method of any one of claims 37 to 55, wherein each RNA comprises a poly-A sequence.

57. The method of claim 55 or 56, wherein the poly-A sequence comprises at least 100 nucleotides.

58. The method of any one of claims 55 to 57, wherein the poly-A sequence comprises or consists of the nucleotide sequence of SEQ ID NO: 15.

59. The method of any one of claims 37 to 58, wherein the RNA is formulated as a liquid, formulated as a solid, or a combination thereof.

60. The method of any one of claims 37 to 59, wherein the RNA is administered by injection.

61. The method of any one of claims 37 to 60, wherein the RNA is administered by intravenous administration.

62. The method of any one of claims 37 to 61, wherein the RNA is formulated as lipid particles.

63. The method of claim 62, wherein the RNA lipid particles are lipid nanoparticles (LNP).

64. The method of any one of claims 37 to 63, wherein the RNA is formulated as a pharmaceutical composition.

65. The method of claim 64, wherein the pharmaceutical composition further comprises one or more pharmaceutically acceptable carriers, diluents and/or excipients.

66. The method of any one of claims 37 to 65, wherein the subject is a human.


SEQUENCE LISTING (17 sequences)

SEQ ID NO: 1 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 133 aa, Homo sapiens [sequence omitted]

SEQ ID NO: 3 - protein, 585 aa, Homo sapiens [sequence omitted]

SEQ ID NO: 4 - protein, 772 aa, Artificial Sequence
  hIL7-hAlb
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGSGGGGSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - RNA, 2801 nt, Artificial Sequence
  hIL7-hAlb
agacgaacuaguauucuucugguccccacagacucagagagaacccgccaccauguuccauguuucuuuuagguauaucuuuggacuuccuccccugauccuuguucuguugccaguagcaucaucugauugugauauugaagguaaagauggcaaacaauaugagaguguucuaauggucagcaucgaucaauuauuggacagcaugaaagaaauugguagcaauugccugaauaaugaauuuaacuuuuuuaaaagacauaucugugaugcuaauaaggaagguauguuuuuauuccgugcugcucgcaaguugaggcaauuucuuaaaaugaauagcacuggugauuuugaucuccacuuauuaaaaguuucagaaggcacaacaauacuguugaacugcacuggccagguuaaaggaagaaaaccagcugcccugggugaagcccaaccaacaaagaguuuggaagaaaauaaaucuuuaaaggaacagaaaaaacugaaugacuuguguuuccuaaagagacuauuacaagagauaaaaacuuguuggaauaaaauuuugaugggcacuaaagaacacggcggcucuggaggaggcggcuccggaggcgaugcacacaagagugagguugcucaucgcuuuaaagauuugggagaagaaaauuucaaagccuugguguugauugccuuugcucaguaucuucagcaguguccauuugaagaucauguaaaauuagugaaugaaguaacugaauuugcaaaaacauguguugcugaugagucagcugaaaauugugacaaaucacuucauacccuuuuuggagacaaauuaugcacaguugcaacacuucgugaaaccuauggugaaauggcugacugcugugcaaaacaagaaccugagagaaaugaaugcuucuugcaacacaaagaugacaacccaaaccucccccgauuggugagaccagagguugaugugaugugcacugcuuuucaugacaaugaagaaacauuuuugaaaaaauacuuauaugaaauugccagaagacauccuuacuuuuaugccccggaacuccuuuucuuugcuaaaagguauaaagcugcuuuuacagaauguugccaagcugcugauaaagcugccugccuguugccaaagcucgaugaacuucgggaugaagggaaggcuucgucugccaaacagagacucaagugugccagucuccaaaaauuuggagaaagagcuuucaaagcaugggcaguagcucgccugagccagagauuucccaaagcugaguuugcagaaguuuccaaguuagugacagaucuuaccaaaguccacacggaaugcugccauggagaucugcuugaaugugcugaugacagggcggaccuugccaaguauaucugugaaaaucaagauucgaucuccaguaaacugaaggaaugcugugaaaaaccacuguuggaaaaaucccacugcauugccgaaguggaaaaugaugagaugccugcugacuugccuucauuagcugcugauuuuguugaaaguaaggauguuugcaaaaacuaugcugaggcaaaggaugucuuccugggcauguuuuuguaugaauaugcaagaaggcauccugauuacucugucgugcugcugcugagacuugccaagacauaugaaaccacucuagagaagugcugugccgcugcagauccucaugaaugcuaugccaaaguguucgaugaauuuaaaccucuuguggaggagccucagaauuuaaucaaacaaaauugugagcuuuuugagcagcuuggagaguacaaauuccagaaugcgcuauuaguucguuacaccaagaaaguaccccaagugucaacuccaacucuuguagaggucucaagaaaccuaggaaaagugggcagcaaauguuguaaacauccugaagcaaaaagaaugcccugugcagaagacuaucuauccgugguccugaaccaguuauguguguugcaugagaaaacgccaguaagugacagagucaccaaaugcugcacagaauccuuggugaacaggcgaccaugcuuuucagcucuggaagucgaugaaacauacguucccaaagaguuuaaugcugaaacauucaccuuccaugcagauauaugcacacuuucugagaaggagagacaaaucaagaaacaaacugcacuuguugagcuggugaaacacaagcccaaggcaacaaaagagcaacugaaagcuguuauggaugauuucgcagcuuuuguagagaagugcugcaaggcugacgauaaggagaccugcuuugccgaggaggguaaaaaacuuguugcugcaagucaagcugccuuaggcuuaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 6 - protein, 752 aa, Artificial Sequence
  hAlb-hIL2
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGGGSGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - RNA, 2741 nt, Artificial Sequence
  hAlb-hIL2
agacgaacuaguauucuucugguccccacagacucagagagaacccgccaccaugaaguggguaaccuuuauuucccuucuuuuucucuuuagcucggcuuauuccagggguguguuucgucgagaugcacacaagagugagguugcucaucgcuuuaaagauuugggagaagaaaauuucaaagccuugguguugauugccuuugcucaguaucuucagcaguguccauuugaagaucauguaaaauuagugaaugaaguaacugaauuugcaaaaacauguguugcugaugagucagcugaaaauugugacaaaucacuucauacccuuuuuggagacaaauuaugcacaguugcaacacuucgugaaaccuauggugaaauggcugacugcugugcaaaacaagaaccugagagaaaugaaugcuucuugcaacacaaagaugacaacccaaaccucccccgauuggugagaccagagguugaugugaugugcacugcuuuucaugacaaugaagaaacauuuuugaaaaaauacuuauaugaaauugccagaagacauccuuacuuuuaugccccggaacuccuuuucuuugcuaaaagguauaaagcugcuuuuacagaauguugccaagcugcugauaaagcugccugccuguugccaaagcucgaugaacuucgggaugaagggaaggcuucgucugccaaacagagacucaagugugccagucuccaaaaauuuggagaaagagcuuucaaagcaugggcaguagcucgccugagccagagauuucccaaagcugaguuugcagaaguuuccaaguuagugacagaucuuaccaaaguccacacggaaugcugccauggagaucugcuugaaugugcugaugacagggcggaccuugccaaguauaucugugaaaaucaagauucgaucuccaguaaacugaaggaaugcugugaaaaaccacuguuggaaaaaucccacugcauugccgaaguggaaaaugaugagaugccugcugacuugccuucauuagcugcugauuuuguugaaaguaaggauguuugcaaaaacuaugcugaggcaaaggaugucuuccugggcauguuuuuguaugaauaugcaagaaggcauccugauuacucugucgugcugcugcugagacuugccaagacauaugaaaccacucuagagaagugcugugccgcugcagauccucaugaaugcuaugccaaaguguucgaugaauuuaaaccucuuguggaggagccucagaauuuaaucaaacaaaauugugagcuuuuugagcagcuuggagaguacaaauuccagaaugcgcuauuaguucguuacaccaagaaaguaccccaagugucaacuccaacucuuguagaggucucaagaaaccuaggaaaagugggcagcaaauguuguaaacauccugaagcaaaaagaaugcccugugcagaagacuaucuauccgugguccugaaccaguuauguguguugcaugagaaaacgccaguaagugacagagucaccaaaugcugcacagaauccuuggugaacaggcgaccaugcuuuucagcucuggaagucgaugaaacauacguucccaaagaguuuaaugcugaaacauucaccuuccaugcagauauaugcacacuuucugagaaggagagacaaaucaagaaacaaacugcacuuguugagcuggugaaacacaagcccaaggcaacaaaagagcaacugaaagcuguuauggaugauuucgcagcuuuuguagagaagugcugcaaggcugacgauaaggagaccugcuuugccgaggaggguaaaaaacuuguugcugcaagucaagcugccuuaggcuuaggcggcucuggaggaggcggcuccggaggcgcuccaacaucuucuucaacaaagaaaacacagcuucagcuugaacaccuucuucuugaucuucagaugauucugaauggaaucaacaauuacaaaaauccaaaacugacaagaaugcugacauuuaaauuuuacaugccaaagaaagcaacagaacugaaacaccuucagugccuugaagaagaacugaaaccucuggaagaagugcugaaucuggcucagagcaaaaauuuucaccugagaccaagagaucugaucagcaacaucaaugugauugugcuggaacugaaaggaucugaaacaacauucaugugugaauaugcugaugaaacagcaacaauuguggaauuucugaacagauggauuacauuuugccagucaaucauuucaacacugacaugaugacucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacaccgagaccugguccagagucgcuagccgcgucgcuaaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 8 - protein, 26 aa, Artificial Sequence
  Sec
MRVMAPRTLILLLSGALALTETWAGS

SEQ ID NO: 9 - protein, 55 aa, Artificial Sequence
  MITD
IVGIVAGLAVLAVVVIGAVVATVMCRRKSSGGKGGSYSQAASSDSAQGSDVSLTA

SEQ ID NO: 10 - protein, 65 aa, Artificial Sequence
  P2P16
KKQYIKANSKFIGITELKKLGGGKRGGGKKMTNSVDDALINSTKIYSYFPSVISKVNQGAQGKKL

SEQ ID NO: 11 - protein, 10 aa, Artificial Sequence
  GS-Linker
GGSGGGGSGG

SEQ ID NO: 12 - protein, 14 aa, Artificial Sequence
  GS-Linker
GSSGGGGSPGGGSS

SEQ ID NO: 13 - RNA, 47 nt, Artificial Sequence
  5′-UTR
aacuaguauucuucugguccccacagacucagagagaacccgccacc

SEQ ID NO: 14 - RNA, 278 nt, Artificial Sequence
  3′-UTR
cugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacc

SEQ ID NO: 15 - RNA, 110 nt, Artificial Sequence
  A30L70
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 16 - protein, 15 aa, Artificial Sequence
  P2
QYIKANSKFIGITEL

SEQ ID NO: 17 - protein, 32 aa, Artificial Sequence
  P17
MTNSVDDALINSTKIYSYFPSVISKVNQGAQG